(19) United States
(12) Patent Application Publication
(10) Pub. No.: US 2024/0032564 A1
(43) Pub. Date: Feb. 1, 2024

(54) METHOD OF MANUFACTURING A FORMED MEAT-REPLACEMENT FOOD PRODUCT AND A FORMED MEAT-REPLACEMENT FOOD PRODUCT COMPRISING A PLANT BASED PROTEINACEOUS BINDER INGREDIENT, A PLANT BASED PROTEINACEOUS BINDER INGREDIENT AND A METHOD FOR MANUFACTURING A PLANT-BASED PROTEINACEOUS BINDER INGREDIENT

(71) Applicant: VALIO OY, Helsinki (FI)

(72) Inventors: Anni Katriina NISKAKOSKI, Helsinki (FI); Zhongqing JIANG, Helsinki (FI); Alejandra KOLEHMAINEN, Kauniainen (FI); Jingwei LIU, Helsinki (FI); Samuel MCCORMICK, Espoo (FI); Maija ITKONEN, Helsinki (FI); Maria Ilona SILVEN, Helsinki (FI)

(21) Appl. No.: 18/258,115

(22) PCT Filed: Dec. 23, 2020
(86) PCT No.: PCT/EP2020/087813

§ 371 Date: Jun. 16, 2023

(51) Int. Cl. A23J 3/14; A23J 3/26; A23J 3/22

(52) U.S. Cl. CPC A23J 3/14; A23J 3/26; A23J 3/227

(57) ABSTRACT

A plant-based proteinaceous binder ingredient that is a disturbed-coagulum can be manufactured with disrupting-coagulation treatment described in the application. The binder ingredient improves the handling properties of intermediate products including textured vegetable proteins manufactured using low-moisture protein texturization extrusion. The resulting end-products (formed meat-replacement food products that are meat imitates) show an improved mouthfeel and are highly resistant to bending and suitable for transport. This is believed to relate to a novel self-encapsulated proteinaceous structure of the binder that is assumed to open when being mixed with a sufficiently high mixing power.

### FIELD OF THE INVENTION

The invention relates, according to a first aspect, generally to the field of food technology, and more particularly to formed meat-replacement food products that comprise as an ingredient textured vegetable proteins (TVPs) manufactured with low moisture protein texturization extrusion that are in suitable particulate/granule, flake/chunk/thread form, and plant-based proteinaceous binder ingredients suitable for the manufacture of formed meat-replacement food products. According to a second aspect, the invention relates generally to self-encapsulated proteinaceous particles, and to their manufacture and use.

### BACKGROUND ART

Vegetables are parts of plants that are consumed by humans or other animals as food, and hereinafter the word vegetable is intended to cover plants collectively and to refer to all edible plant matter, including the flowers, fruits, stems, leaves, roots, and seeds.

In the recent years, many people have turned vegetarian or vegan, or at least increased the share of plant-based products, vegetables and vegetable products in their diet.

While ecological concerns are the reason for some, it appears also clear that plant-based products, vegetables and products made of vegetables should be a central part of a healthy diet. Many consumers find it difficult to ensure a daily protein intake with plant-based products, vegetables or products made of vegetables, while some find it time-consuming to prepare the protein-containing ingredients for cooking or baking.

Thus, there is a market for vegetarian or vegan foods produced on an industrial basis by extrusion cooking. Extrusion cooking is a continuous process which enables the production of texturized proteins that are unique products made by extrusion. The extrusion enables controlling the functional properties such as density, rate and time of rehydration, shape, product appearance and mouthfeel.

Hereinafter, textured vegetable proteins are “products that have been transformed from a substantially flour-type or particulate material into one which has a meat-like texture. The resulting textured vegetable protein product provides chewiness and fibrous character.” Textured vegetable proteins can be further characterized as ‘food products made from edible protein sources and characterised by having structural integrity and identifiable texture such that each unit will withstand hydration in cooking and other procedures used in preparing the food for consumption’, as defined in cited reference [1]. Though soy is a commonly used base material for textured vegetable proteins, legumes (such as peas, alfalfa, clover, beans, peas, chickpeas, lentils, lupins and peanuts, fava beans, mung beans), cottonseed, corn, wheat, oat, barley, rye, rice and similar proteins can be texturized, to qualify as textured vegetable proteins. However, and importantly, textured plant-based proteins that do not withstand hydration in cooking do not qualify as textured vegetable proteins.

For extrusion of meat analogues or texturized vegetable proteins (both of which are hereinafter used synonymously), a twin screw extruder is normally used. There are mainly two types of extrusion cooking methods for preparing meat replacement products.

One kind of texturized vegetable proteins is produced with low moisture protein texturization extrusion. Such products have a moisture content between 10% and 40% after extrusion (moisture content during extrusion is between 15% and 40%). They often have a sponge-like texture and require rehydration prior consumption. These products are often used as minced meat substitutes or extenders in meat products but can hardly mimic fibrous whole-muscle meat.

Another kind of texturized vegetable proteins is manufactured with high moisture protein texturization extrusion. Such products have a moisture content between 40% and 80% (moisture content during extrusion is higher than in low-moisture protein texturization extrusion). They generally resemble more muscle food than the texturized vegetable products manufactured with low moisture texturization extrusion.

Texturized vegetable proteins are generally manufactured by mixing at least one proteinaceous matrix forming ingredient, such as protein isolate or protein concentrate (that generally are referred to as protein fractions), possibly starch-containing particles, possibly oil, possibly color, possibly flavoring, possibly stabilizer, mixing the ingredients into a slurry, and extruding the ingredients mixed to a slurry in an extruder that is configured to carry out protein texturization extrusion.

Textured vegetable proteins manufactured using a low moisture protein texturization extrusion are being increasingly used in particulate/granule/flake/chunk/thread form as an ingredient in the manufacture of formed meat-replacement food products, which are after forming cooked, fried, grilled or baked. The formed meat-replacement food product may have a flat, generally round or oval, shape, or disc-like shape (the formed meat-replacement food product is in these situations a vegetarian/vegan meat-replacement patty), or a ball shape (the formed meat-replacement food product is in this situation a vegetarian/vegan meat-replacement ball), but the formed meat-replacement food product may alternatively have any other suitable shape, like sausage, nugget, fish fingers, or ham, for example. The ingredients may be placed inside a pastry crust that is then baked or fried. Or alternatively, the formed meat-replacement food products may be breaded, then baked or fried.

When textured vegetable proteins manufactured using a low moisture protein texturization extrusion method are used in suitable particulate/granule/flake/chunk/thread form as an ingredient for a meat-replacement formed food product, a proteinaceous binder is necessary.

### Objective of the Invention

Egg protein, meat protein and bovid milk protein (such as whey protein or other milk protein) have a good ability of heat-induced gelation, so they could be easily used as a binder for formed food products (such as meat patties, meat balls, meat loaf, cheese, egg gel products etc.). However, they are unsuitable for vegans (who avail themselves of diets devoid of any animal-based ingredients) or vegetarians (such as those who avail themselves of a non-meat, non-poultry, no-egg diet and do not consume milk, some of which however allow themselves to consume butter, though, and some allow themselves to consume bovid milk) and thus do not qualify as a plant-based proteinaceous binder ingredient for a formed meat-replacement food product. More importantly, for ecological, sustainability, economical and diversity perspectives, it is favorable to enable technologies that can make a proteinaceous binder using plant-based ingredients.

Soy protein is generally suitable to be used as a binder, but soy is considered as a common allergen and is a problem for many consumers. Further, many consumers have concerns about GMO soybeans. As an alternative, wheat gluten could be used as a binder, but wheat is considered as a common allergen too. Furthermore, wheat is a serious problem for those with the coeliac disease. Thus, in the context of the planned use of the plant-based binder ingredient, the binder ingredient is devoid of wheat based proteinaceous materials, in particular of wheat gluten. Optionally, the binder ingredient may be devoid of soy based proteinaceous materials, in particular of soy protein.

Wheat and soy have been dominating plant-based meat-replacement food market for many years for various reasons. First of all, they have a long history of more matured cultivation and are bulk produced in massive quantities, which support their broadness of supply, quality and production cost-effectiveness. Additionally, and equally crucially, wheat gluten and soy proteins have unique and excellent protein functionalities, such as in terms of gelling and binding. Wheat gluten is the only protein that can form an elastic dough. Other plant-based proteins are generally more challenging to be used as proteinaceous binders compared to soy proteins and wheat gluten. The differences between non-soy plant-based proteins and soy proteins have been scientifically analyzed and proven to be related to poorer solubility and gelling properties of non-soya proteins. More fundamentally, soy protein and wheat gluten have both a very unique molecular structure, hydrophobicity-hydrophilicity ratio and disulfide bond forming amino acid side chains.

Methylcellulose (which is a food additive approved in Europe by the European Food Safety Authority under E number E461) can be used as a binder for formed meat-replacement food products. Such products generally have adequate structural properties at least regarding cohesiveness, elasticity and intactness against pressing or bending. Methylcellulose, though, is synthetically produced by heating cellulose with a caustic solution and treating it with methyl chloride. A substitution reaction follows, in which the hydroxyl residues are replaced by methoxide.

A better alternative to methylcellulose is thus desirable in order to offer to the consumer more natural foods, or at least foods, the ingredients of which are less synthetic. It is even more desirable to have the ingredients as proteinaceous ingredients, since proteins are essential nutrients, and are especially crucial for consumers who mainly avail themselves of a vegetarian diet and consequently have a higher risk of protein intake insufficiency. However, the alternative binder ingredient should enable improving certain structural properties in the meat-replacement formed food product, in particular one or more of the following structural properties: cohesiveness, elasticity and intactness against pressing or bending.

Further formed meat-replacement products which do not contain methylcellulose that are available on the market and known to the inventors are dry, crumbling, soft, dough-like and easy to break by pressing or bending.

The inventors have discovered a manufacturing method for a new kind of plant-based proteinaceous binder ingredient. In the method and the new kind of plant-based proteinaceous binder ingredient, neither methyl cellulose nor wheat gluten is used. Optionally, soy and soy proteins may be excluded but the method and plant-based binder ingredient we have invented works also if soy is used. The plant-based proteinaceous binder is suitable for improving certain structural properties of formed meat-replacement food products. Depending on the selection of the lipids, the binder ingredient can be made suitable for vegetarians or vegans.

Thus, it is a first objective of the invention to improve the structural properties of formed meat-replacement food products devoid of methylcellulose and wheat based proteinaceous materials, in particular wheat gluten, and optionally also devoid of soy and soy based proteinaceous materials, in particular soy protein. This objective can be met with the method of manufacturing a formed meat-replacement food product according to independent claim 1 and with a formed meat-replacement food product according to parallel independent claim 17.

A second objective of the invention is to offer an alternative for methylcellulose and wheat based proteinaceous ingredients (in particular, wheat gluten and optionally also soy based proteinaceous ingredients, in particular soy protein) as plant-based proteinaceous binder ingredient.

This second objective can be met with the method for manufacturing a plant-based binder ingredient according to parallel independent claim 20 and with a plant-based proteinaceous binder ingredient according to parallel independent claim 37.

Further, the inventors have discovered a manufacturing method that produces self-encapsulated proteinaceous particles. The proteinaceous particles have advantageous properties and may be used, in addition to being used as a proteinaceous binder ingredient, also for other purposes.

The method for manufacturing self-encapsulated proteinaceous particles is disclosed in independent claim 47. Self-encapsulated proteinaceous particles are disclosed in claims 53 and 55. They can be manufactured with the methods disclosed in claims 47 and 20.

Independent claims 57 and 59 disclose novel methods for using for self-encapsulated proteinaceous particles.

A further objective of the invention is to improve the handling properties of intermediate products between the shaping and cooking steps in industrial manufacturing process of formed meat-replacement products. This objective can be achieved with the method disclosed in the parallel independent claim 61.

An even further objective of the invention is to improve the stability of self-encapsulated proteinaceous particles. This objective can be achieved with the method disclosed in the parallel independent claim 62.

The dependent claims describe advantageous aspects of the methods, of the formed meat-replacement food product, and of the plant-based proteinaceous binder ingredient, of the manufacturing method of the self-encapsulated proteinaceous particles and of the self-encapsulated proteinaceous particles, and of the method of improving the stability of self-encapsulated proteinaceous particles, and of the further methods.

### Advantages of the Invention

The inventors have surprisingly found out that industrial manufacturing process of formed meat replacement products can be improved by using plant-based proteinaceous binder ingredient that is or comprises a disturbed-coagulum. So, it is possible to improve certain structural properties in intermediate products between the shaping step and cooking step which, in consequence, makes the intermediate products better to withstand the fast movements in the industrial processes (vibrations, bending and shocks in industrial processes), which further makes it possible to reduce the need to re-work the intermediate products. The intermediate products may thus be better suited for transport, since they may be made more durable against vibrations and shocks that are caused by conveyors and transport. Furthermore, and at times even more importantly, the mouthfeel of the resulting end-products (formed meat-replacement products) can be improved by using a plant-based proteinaceous binder ingredient that is or comprises a disturbed-coagulum.

According to a further aspect of the invention, the method of manufacturing a formed meat-replacement food product comprises the steps of:
a) preparing a mixture of:
   i) textured vegetable proteins manufactured using a low moisture protein texturization extrusion method that preferably have a sponge-like texture and advantageously are in particulate/granule/flake/chunk/thread form,
   ii) a plant-based proteinaceous binder ingredient—however excluding wheat based proteinaceous ingredients and methylcellulose, optionally also soy based proteinaceous ingredients—which is or comprises a disturbed-coagulum that is a dispersion of a weak gel of an emulsion of:
      a. water/water-containing liquid and lipid that preferably is fat/oil/margarine, most preferably vegetable oil/fat/margarine, advantageously in particular comprising at least one of the following: oilseed oil, rapeseed oil, field mustard oil, olive oil, coconut fat, cocoa fat,
      b. possible further ingredients, and
      c. at least one plant based—however excluding wheat and optionally also soy based proteinaceous ingredients—proteinaceous ingredient with the proteinaceous portion making at least 6.1%, preferably between 6.1% to 15%, most preferably between 7% and 15% by weight of the binder ingredient and
   iii) possible further ingredients, such as at least one of the following: boiled/baked/powdered/raw potato, onion, tomato, herbs, spices, salt, breadcrumbs, starches, for example, that preferably are dried—however excluding methylcellulose;
b) shaping the mixture into desired form; and
c) processing—cooking, frying, grilling, steaming and/or baking, for example—the shaped mixture such that the binder ingredient undergoes further gelation under non-melting conditions for the proteins.

Instead of the expression “disturbed-coagulum”, or in addition to it, we also considered alternative expressions, “disturbed-curd”, “disturbed-clot” and “disturbed-coagulate” which could be used. Hereinafter, however, we disclose the invention using the expression “disturbed-coagulum”. A coagulum can be a clot or a curd. A clot is defined as “soft, thickened area or lump formed on or within a liquid; a thick or jumbled mass or cluster; agglomeration”. A curd is “the coagulated part of milk, from which cheese is made: it is formed when milk sours and is distinguished from whey, the watery part”; in the context of the present invention, the expression “curd” is used for coagulum of plant-based materials, such as plant-based milk. Our materials before and after disrupting of the coagulation are often more like a “clot”, especially, before the disruption, the materials are often clot, however they do not necessarily generate whey. It is not a strong curd because the acidification level was controlled to be relatively low.

The inventors have found out that surprisingly, with a plant-based proteinaceous binder ingredient that is or comprises a disturbed-coagulum, it is possible
(a) to improve the shape intactness and shape stability of shaped-but-not-yet-cooked meat-replacement food products (such as, during the production or if offered as a semi-finished product), and
(b) to improve structural properties of shaped-and-cooked (formed) meat-replacement food products, preferably at least cohesiveness, elasticity and resilience against pressing or bending.

At the time of writing, we have a preliminary scientific explanation for this phenomenon, and we suspect that disturbed-coagulum is a unique suspension system containing self-encapsulated proteinaceous particles that introduces a specific bonding structure that is particularly well suitable to bond pieces of textured vegetable proteins manufactured with low-moisture protein texturization extrusion to other pieces. “Pieces” refers to suitable particulate/granule, flake, thread or chunk form. While the protein-protein interaction within each piece of textured vegetable protein (i.e. between protein bundles) is already formed during extrusion, the suspension system appears to function particularly well to create a bonding between pieces that gives a specific mouthfeel and improves the handling properties of the intermediate products and end-product (formed meat-replacement product).

Preferably, between the mixing and shaping steps, the mixture is kneaded. We expect that this is capable to ensure sufficient unfolding of the self-encapsulated particles from the proteinaceous binder ingredients, to ensure sufficient development of protein-protein interaction and bonding, and to ensure a more uniform distribution of the bonding which makes it possible to further improve the structural properties mentioned in the preceding paragraph.

Preferably, the plant-based proteinaceous binder ingredient comprises or is in the form of protein colloid, protein extract, protein isolate, protein concentrate or a mixture of at least two of these.

Advantageously, the plant-based proteinaceous binder ingredient comprises, is composed of, or substantially consists of pea proteins, fava bean proteins, mung bean proteins, chickpea proteins, or any combination thereof.

Advantageously, the plant-based proteinaceous ingredient comprises, is composed of, or substantially consists of protein fractions having isoelectric points between 4 and 5, preferably approximately at 4.5.

Alternatively, or in addition to this, the plant-based proteinaceous ingredient may comprise, be composed of, or substantially consist of protein fractions having water solubility being affected by pH, and the lowest solubility is preferably at pH approximately between 4 and 5, preferably near 4.5.

Preferably, the plant-based proteinaceous binder ingredient comprises, is composed of, or substantially consists of protein fractions having gel forming properties after being denatured and acidified to pH between 3.5 and 6.0, preferably between 4.0 and 5.9, most preferably between 4.5 and 5.5, or to 0.5-1.4 in the pH scale above the isoelectric point of the at least one plant-based proteinaceous ingredient.

Preferably, the plant-based proteinaceous binder ingredient comprises materials of or is made of materials from: cereal grains (excluding wheat), legume crops (optionally: excluding soy), vegetable crops, pulse grains, oilseeds (optionally: excluding soy) and/or seeds for sowing forage. In particular, the cereal grains may be or comprise at least one of the following: oat, barley, rye, rice, corn. The pulse grains/legume crops/vegetable crops/oilseeds/seeds for sowing forage may be or comprise at least one of the following: peas, beans, green peas, peanuts, chickpeas, fava beans, lentils, kidney beans, white beans, black beans, black eye beans, mung beans, and optionally soy beans, alfalfa. The proteins of pea and fava bean naturally and substantially comprise proteins having isoelectric points between 4 and 5, mostly around 4.5. The proteins of cereal grains such as oat, barley, rye, rice and corn can be treated by enzymatic modification, such as, protein-glutaminase induced deamidation reaction to deamidation degree approximately between 50% and 70%, to achieve a status that the proteins substantially comprise proteins having isoelectric points between 3.5 and 6.0, preferably between 4.0 and 5.5, most preferably between 4.5 and 5.4. Such deamidation treatment method can be found in [2].

Advantageously, in the mixing step, also a protein crosslinking/binding enzyme, such as transglutaminase, may be included. If so, the mixing is preferably then carried out below 40° C., preferably between 1° C. and 25° C., to avoid that the enzyme reacts too fast and earlier than the shaping step or to reduce the reacting of the enzyme before the shaping step. The inventors have observed that formed meat-replacement food products show a remarkably increased elasticity/bending ability if the plant-based proteinaceous binder ingredient that is or comprises a disturbed-coagulum is combined with the crosslinking/binding enzyme. Thus, it appears to be possible to obtain a firm but however elastic structure in the formed meat-replacement food product that exceeds the elasticity/bending ability of any formed meat-replacement food product patty, in particular, known to the inventors. We have a video recording of a thin patty that after frying on a pan remained on a spatula (turner) and is flapping. As regards its elasticity/bending ability, it resembles eggs sunny side up. This kind of property is particularly desirable since it makes the formed meat-replacement food products particularly appealing in mouthfeel and cooking-related user experience, and suitable for transport: if the transport container, such as plastic package, is vibrated or dropped, the formed meat-replacement food products are less likely to break.

Further, the time between the mixing step and the shaping steps may then preferably be less than 4 h, preferably less than 1 h, more preferably less than 30 min to have the mixing and shaping steps before the enzyme has reacted to an unsuitable extent.

When the enzyme is used, between the mixing and the shaping steps, the temperature of the mixture is preferably kept below 40° C., preferably between 1° C. and 25° C.; the mixture may be in a storing, buffering and/or transferring environment between the mixing step and the shaping step.

Further, with the enzyme, the shaped mixture is preferably subjected to an incubation step, in which the shaped product is preferably
i) incubated at temperature above 30° C., preferably between 40° C. and 50° C., for a time between 30 min and 4 hour, preferably between 1 hour and 2 hour, or
ii) incubated at temperature between 1° C. and 6° C., for a time between 4 h and 36 h, preferably between 8 h and 16 h.

In such a situation, the processing step is preferably a direct continuation to the incubation step, such as after heating the shaped patty in an autoclave oven to an incubation temperature, such as temperature above 30° C., preferably between 40 and 50° C., for a suitable incubation time, such as between 30 min and 4 hour, then directly further increasing the temperature to the processing temperature, such as 115° C.

According to the second aspect of the invention, the formed meat-replacement food product is manufactured using the method according to the first aspect of the invention.

The inventors have found out that surprisingly, formed meat-replacement food products manufactured using a plant-based proteinaceous binder ingredient that is or comprises a disturbed-coagulum, it is possible to improve structural properties of formed meat-replacement food products, preferably at least cohesiveness, elasticity and resilience against pressing or bending. Formed meat-replacement food products may thus be better suitable for transport since they are more durable against vibrations and shocks that can be caused in transport.

Preferably, the formed meat-replacement food product is devoid of gluten, egg protein, meat protein, dairy, methylcellulose, wheat and optionally also soy.

In particular, the formed meat replacement food product may be a vegetarian/vegan meat replacement patty, a vegetarian/vegan meat replacement ball, a vegetarian/vegan meat replacement sausage, a vegetarian/vegan meat replacement fish finger, a vegetarian/vegan meat replacement ham, or a vegetarian/vegan meat replacement nugget.

Preferably, the protein crosslinking/binding enzyme (such as transglutaminase) is added at or after the mixing step (K1, K9 in FIG. 1) in which the self-encapsulated proteinaceous particles in the plant-based binder ingredient are opened when the mixing is carried out with a sufficient intensity or power such that the opening described below in the detailed description takes place. With a view on an industrial-scale production, with the inventors' current knowledge, it is preferable to add the protein crosslinking/binding enzyme at the mixing step. If protein crosslinking/binding enzyme is combined with the plant-based proteinaceous binder ingredient alone, the reaction may be too hard to stop. This can easily lead to a degradation of quality of the plant-based proteinaceous binder ingredient before it can be mixed with the TVP such that the quality of the end-product may suffer.

According to a further aspect of the invention, the method for manufacturing a plant-based proteinaceous binder ingredient comprises the steps of:
Mixing an emulsion of
   water or water-based liquid,
   lipid that preferably is fat/oil/margarine, most preferably vegetable oil/fat/margarine, advantageously in particular comprising at least one of the following: oilseed oil, rapeseed oil, field mustard oil, olive oil, coconut fat, cocoa fat,
   possible further ingredients, and
   at least 6.1%, preferably between 6.1% to 15%, most preferably between 7% and 15% by weight of at least one plant based proteinaceous ingredient—however excluding wheat based proteinaceous ingredients and methylcellulose and optionally also soy-based proteinaceous ingredients—that comprises, is composed of, or substantially consists of at least one protein colloid/extract/isolate/concentrate or a mixture of at least two of these;
Converting the mixture to a gel or a weak gel by heating and a coagulation-causing treatment, preferably with acidifying in such a manner that its pH is between 5.0 and 5.9 or 0.5-1.4 in the pH scale above the isoelectric point of the at least one plant-based proteinaceous ingredient;
Disrupting the coagulation before the coagulation is completed to obtain disturbed-coagulum. The disrupting of coagulation is carried out to break protein clusters in the gel by cluster-disrupting mixing, stirring or kneading, such as with spatula or whisk during the coagulation. We have found out that disrupted-coagulation can conveniently be achieved by disrupting with limited intensity and/or duration such that the disrupting is ended before disrupted coagulation goes over to homogenization. There may exist further ways to achieve disturbed-coagulum, though. The aim is to achieve disturbed-coagulum that is devoid of homogenization or micronization (micronization is a term usually used to describe size reduction technique that reduces size of particles to less than 10 μm).

With “weak gel”, we mean a gel which under large deformation, enough for the conventional gels to rupture, flows but not ruptures. Without willing to be bound by theory, the weak gel-type rheological properties in dispersions could be due to a sufficiently long relaxation time of topological entanglements among double-helical conformers but not due to the formation of a three-dimensionally percolated permanent network.

The inventors have surprisingly found that the disrupted-coagulation treatment changes the binding properties of the proteinaceous binder ingredient in such a manner that it generates disturbed-but-not-homogenized-coagulum which functions as a unique suspension system containing self-encapsulated proteinaceous particles that introduces a specific bonding structure that is particularly well responsive to the bonding within textured vegetable proteins manufactured with low-moisture protein texturization extrusion manufacturing. This enables the manufacture of formed meat-replacement food products that show improved structural characteristics, in particular resistance to bending and elasticity. With regard to the mouthfeel, certain gel-like bite experience is the expected value in meat alternatives. With the new binder ingredient, the formed meat-replacement meat products (that are meat analogues) can be made with a superior mouthfeel mimicking meat, and with pleasant bite resistance.

Preferably, the disruption of the coagulation is carried out to break protein clusters in the gel by mixing or kneading, such as with spatula or whisk during the coagulation. The disruption of the coagulation should be, preferably, suitably low-energetic. The disruption should preferably not be carried out by homogenization or micronization, such as with milling, high-pressure homogenizer, high-speed (such as rotating speed above 1000 rpm, revolutions-per-minute, more normally above 10 000 rpm) Rotor Stator disperser homogenizer, high-speed disperser, microfluidizer, fruit juice (or soup, or puree) blender equipped with rotating blade, yogurt smoothing filter nozzle equipped with sieve pore size smaller than 500 μm.

The coagulation-causing treatment may comprise the step, or consist of the step, of adding acid to achieve pH between 5.0 and 5.9, at temperature between 50° C. and 100° C., preferably between 65° C. and 100° C., which is carried under cluster-disrupting (coagulation-disrupting) dispersion.

Alternatively, the coagulation-causing treatment may comprise the steps, or consist of the steps, of adding acid to achieve pH between 5.0 and 5.9, at temperature between 50° C. and 100° C., preferably between 65° C. and 100° C., followed by cluster-disrupting (coagulation-disrupting) dispersion before the proteinaceous mixture coagulation is substantially completed, which is within 24 h, preferably no longer than 4 h, more preferably within 1 h; and before the mixture is chilled to below 10° C., preferably before the mixture is chilled to below 20° C.

The coagulation after acid addition is a continuous process that takes place in a certain amount of time, for example, in 25 h, more often in 5 h, even more often in 1.5 h. When coagulation is ongoing the gel strength (such as hardness in texture analysis) of the proteinaceous mixture that undergoes coagulation process will constantly increase with time after acid addition (for example, from 0 h to 25 h), together with the decrease in temperature after acid addition (for example, from 50° C. to 10° C.). The coagulation will be substantially completed, when the gel strength of the proteinaceous mixture reaches a plateau or global maximum, which will also be the highest level of gel strength that the non-disturbed coagulated proteinaceous mixture will reach.

Advantageously, in the disrupting (clusterization-disrupting, or coagulum-disturbing) step, the size of any large gel clusters is controlled-reduced in size to a predetermined range. In particular, any gel clusters having a size of at least 10 mm, preferably of between 10 mm and 200 mm, may be reduced by dispersing in size substantially to size larger than 0.101 mm and smaller than 10 mm, preferably between 0.3 mm and 5 mm, more preferably between 0.3 mm and 2 mm.

The coagulation-causing treatment may include the step of adding vinegar (preferably white vinegar, or white wine vinegar), citrus fruit juice, acetic acid, lactic acid, or any weak acid with a pKa value between 2.0 and 5.1, preferably between 4.0 and 5.1, more preferably between 4.5 and 4.8, a substance substantially containing a weak acid which has a pKa value between 2.0 and 5.1, preferably between 4.0 and 5.1, more preferably between 4.5 and 4.8, or any mixture thereof. Alternatively, or in addition, any edible substance substantially containing a weak acid which has a pKa value between 2.0 and 5.1, preferably between 4.0 and 5.1, more preferably between 4.5 and 4.8 may be used, or any mixture thereof.

The at least one plant-based proteinaceous ingredient may comprise, be composed of, or substantially consist of protein fractions having isoelectric points between 4 and 5, preferably at approximately 4.5.

The at least one plant-based proteinaceous ingredient may comprise, be composed of, or substantially consist of protein fractions having water solubility being affected by pH, and the lowest solubility is at pH approximately between 4 and 5, preferably near 4.5.

The plant-based proteinaceous binder ingredient is preferably a suspension having pH between 5.0 and 5.9.

The at least one plant-based proteinaceous ingredient may comprise, be composed of, or substantially consist of protein fractions having gel forming properties after being denatured and acidified to pH between 3.5 and 6.0, preferably between 4.0 and 5.9, most preferably between 4.5 and 5.5. Or to 0.5-1.4 in the pH scale above the isoelectric point of the at least one plant-based proteinaceous ingredient.

The plant-based proteinaceous ingredient may comprise materials of or be of materials from: cereal grains (excluding wheat), legume crops (optionally: excluding soy), vegetable crops, pulse grains, oilseeds (optionally excluding soy) and/or seeds for sowing forage. In particular, the cereal grains may be or comprise at least one of the following: oat, barley, rye, rice, and corn. The pulse grains/legume crops/vegetable crops/oilseeds/seeds for sowing forage may be or comprise at least one of the following: peas, beans, green peas, peanuts, chickpeas, fava beans, lentils, kidney beans, white beans, black beans, black eye beans, mung beans, soy, alfalfa.

The method further comprises the step of adding a protein crosslinking/binding enzyme, such as transglutaminase. The surprising advantageous aspect resulting therefrom has been discussed above.

Preferably, the protein crosslinking/binding enzyme is added at or after the disrupting step.

After or at the disrupting step, the disturbed-coagulum may be mixed with water-absorbing materials that preferably are dry materials, to change the consistency to paste-like. A paste-like consistency is particularly suitable for transport. If sold as a ready product, having a paste-like consistency may increase the shelf-life of the product.

According to a further aspect of the invention, a plant-based proteinaceous binder ingredient (which preferably is manufactured with a method according to the third aspect of the invention) comprises a disturbed-coagulum of

an emulsion of:
water or water-based liquid and lipid that preferably is fat/oil/margarine, most preferably vegetable oil/fat/margarine, advantageously in particular comprising at least one of the following: oilseed oil, rapeseed oil, field mustard oil, olive oil, coconut fat, cocoa fat,
possible further ingredients, and
at least 6.1%, preferably between 6.1% to 15%, most preferably between 7% and 15% by weight of at least one plant based proteinaceous ingredient—however excluding wheat-based proteinaceous ingredients and methylcellulose and optionally also soy-based proteinaceous ingredient—that comprises, is composed of, or substantially consists of at least one protein colloid/extract/isolate/concentrate/a mixture of at least two of these.

The inventors have surprisingly found that disturbing the proteinaceous binder to a disturbed-coagulum in the proteinaceous binder ingredient changes the binding properties of the proteinaceous binder ingredient in such a manner that it generates disturbed-but-not-homogenized-coagulum as a unique suspension system containing self-capsulated proteinaceous particles that introduce a specific bonding structure that is particularly well responsive to the bonding within textured vegetable proteins manufactured with low-moisture protein texturization extrusion manufacturing, which further enables manufacturing of formed meat-replacement food products (that are meat analogues) that show improved structural characteristics, in particular resistance to bending and elasticity.

Another discovery was that stability of the self-encapsulated proteinaceous particles was increased when the proteinaceous binder was produced as an emulsion (with lipids) compared to as a solution (no lipids). In the absence of lipids, a significant syneresis was observed during storage of the disturbed-but-not-homogenized-coagulum. Also, shaping the formed meat-replacement products was easier when lipids were present in the proteinaceous binder. While it did not seem to make a difference during the sensory trials, the presence of lipid (preferably a suitable oil/fat/margarine, most preferably vegetable oil/fat/margarine, advantageously in particular comprising at least one of the following: oilseed oil, rapeseed oil, field mustard oil, olive oil, coconut fat, cocoa fat) in the proteinaceous binder is beneficial and may actually be required for the stability and easiness of the raw formed meat-replacement product production process.

Substantially all gel clusters of the gel or weak gel after the disruption have size larger than 0.101 mm and smaller than 10 mm, preferably between 0.3 mm and 5 mm, more preferably between 0.3 mm and 2 mm.

The at least one plant-based proteinaceous ingredient may comprise, be composed of, or substantially consist of protein fractions having isoelectric points between 4 and 5, preferably at approximately 4.5.

The at least one plant-based proteinaceous ingredient may comprise, be composed of, or substantially consist of protein fractions having water solubility being affected by pH, and the lowest solubility is at pH approximately between 4 and 5, preferably at approximately 4.5.

The at least one plant-based proteinaceous ingredient may comprise, be composed of, or substantially consist of protein fractions having gel forming properties after being denatured and acidified to pH between 3.5 and 6.0, preferably between 4.0 and 5.9, most preferably between 4.5 and 5.5, or to 0.5-1.4 in the pH scale above the isoelectric point of the at least one plant-based proteinaceous ingredient.

The plant-based proteinaceous ingredient may comprise or be from: cereal grains (excluding wheat), legume crops (optionally: excluding soy), vegetable crops, pulse grains, oilseeds (optionally excluding soy) and/or seeds for sowing forage. In particular, the cereal grains may be or comprise at least one of the following: oat, barley, rye, rice, and corn. The pulse grains/legume crops/vegetable crops/oilseeds/seeds for sowing forage may be or comprise at least one of the following: peas, beans, green peas, peanuts, chickpeas, fava beans, lentils, kidney beans, white beans, black beans, black eye beans, mung beans, soy beans, alfalfa.

The at least one proteinaceous binder ingredient is preferably incompletely coagulated by protein denaturation, such as heating in an oven or autoclave, for example, followed by acidification mixing with vinegar (preferably white vinegar, or white wine vinegar), citrus fruit juice, acetic acid, lactic acid, or any weak acid with a pKa value between 2.0 and 5.1, preferably between 4.0 and 5.1, more preferably between 4.5 and 4.8, a substance substantially containing a weak acid which has a pKa value between 2.0 and 5.1, preferably between 4.0 and 5.1, more preferably between 4.5 and 4.8, or any mixture thereof, for example.

Self-encapsulated proteinaceous particles can be manufactured by:
Denaturizing proteinaceous particles in a suspension that is an emulsion containing i) lipids which preferably are plant-based, ii) water or any liquid substantially consisting of water, and iii) at least one plant-based proteinaceous material, however preferably excluding methylcellulose;
Inducing gelling or weak gelling in the suspension; and
Disrupting the coagulation to obtain disturbed-coagulum before the coagulation is completed, such that self-encapsulated protein particles are formed.

Self-encapsulated proteinaceous particles are, comprise or consist of gel clusters having a particle size larger than 0.101 mm and smaller than 10 mm, preferably between 0.3 mm and 5 mm, mostly between 0.3 mm and 2 mm.

There are advantages of the suspension containing self-encapsulated particles, especially preferably having pH between 5.0 and 5.9, or 0.5-1.4 in the pH scale above the isoelectric point of the at least one plant based proteinaceous ingredient. Such advantages include:
(a) they help to make good quality end-product and good quality intermediate products for plant-based formed meat-replacement products;
(b) they have good and controllable binding properties that forms good binding under certain mixing or kneading forces, and stay stable when is not mixed or kneaded, so substantially free from aging problems like caking, aggregating, and show a reduced water syneresis;
(c) they have good phase stability, low water syneresis, suspension containing self-encapsulated binder particles made by disrupted coagulation have good phase stability, that has no or little sediment, no or little water (whey) syneresis (e.g. less than 20%, preferably less than 10%, more preferably less than 5%). Nevertheless, there are visible water phase surrounding the self-encapsulated binder particles, in visual observation, the water phase is clear and transparent. If Suspension containing self-encapsulated binder particles is further suspended in water, e.g. in 1:1 ratio, the new mixture (suspension-water mixture) can keep stable non-separating phases for 12 hours or longer, no sedimentation of particles.
(d) they have good cohesiveness against compression, and/or they are substantial free-flowing suspension, so they are not fragile, easier to transport without being worried about the breakage of the gel structure. Their cohesiveness values are above 60%, when they are compressed to a strain between 10% and 65%.
(e) they are a substantially free-flowing suspension, and are substantially homogenous and stable, so they can be easily contained, stored, transported, and unloaded partially or fully for industrial usage.
(f) they have good ability to emulsify or encapsulate oil, such as in a ratio of 1:1. When suspension containing self-encapsulated particles, especially preferably having pH between 5.0 and 5.9, or 0.5-1.4 in the pH scale above the isoelectric point of the at least one plant based proteinaceous ingredient was blended together with plant oil in a 1:1 ratio for 30 s with a (kitchen-use) blender, the resulted mixture can stay stable without phase separation for a few days or longer, or even can stay stable without phase separation after being centrifuged for 10 min at centrifugal force of 644 g (relative centrifugal force), for example.

### LIST OF DRAWINGS

In the following, the invention will be described in more detail by way of detailed embodiments and explanation of a theoretical model. The embodiments, experimental methods and results are explained with reference to appended drawings in FIG. 1 to FIG. 18, of which:

FIG. 1 shows Manufacturing processes for manufacturing the proteinaceous binder ingredient and self-encapsulated proteinaceous particles. It also shows how the proteinaceous binder ingredient and self-encapsulated proteinaceous particles in a suspension can be used in the manufacture of formed meat-replacement products;

FIG. 2 show structures of non-disturbed curd-like gels made with the non-disrupted-coagulation process, of proteinaceous binder ingredient, and of coagulated and over-disrupted suspension;

FIG. 3A shows the proteinaceous binder ingredient (Sample #1), in a cup;

FIG. 3B shows the proteinaceous binder ingredient of FIG. 3A, on a spoon, in a greater detail;

FIG. 4 illustrates the compressing system including the detection probe, base cup-and-platform and sample inside the cup;

FIG. 5 illustrates the three-position compression bending test system;

FIG. 6A is a photograph of a cooked patty of Sample #1T (which corresponds also to Sample #12) during the three-position compression bending test;

FIG. 6B is a photograph of a Reference #Beef during the three-position compression bending test. Reference #Beef was a cooked beef meat patty which was used as our reference sample;

FIG. 7 illustrates the compressing system including the detection probe, base flat platform and sample on top of the platform. The samples were cut into cubic cuboid shape before the analysis;

FIG. 8A, 8B microscopic images of Theory test sample T #1B, observed with an optical microscope equipped with a lens having 5 times magnification;

FIG. 8C microscopic image of Theory test sample T #1B, observed with an optical microscope using 10 times magnification

FIG. 8D microscopic image of Theory test sample T #1D, observed with an optical microscope using 10 times magnification;

FIG. 9A is a schematic representation of a typical plant protein molecule in its native (not denaturized) state;

FIG. 9B illustrates a typical heat denaturized plant protein molecule;

FIG. 9C illustrates a typical plant protein heat-plus-acid-induced non-disrupted coagulation initiation process forming a highly ordered broad continuous network structure;

FIG. 9D, 9E self-encapsulated proteinaceous particles (FIG. 9D is magnification of FIG. 9E);

FIG. 10 a suspension containing self-capsulated proteinaceous particles and water;

FIG. 11A theoretical model for the poor binding property observed;

FIG. 11B theoretical model for the good binding property observed; formation of a TVP-particle-particle-TVP sandwich structure;

FIG. 12 theoretical model illustrating homogenized particles that resulted from homogenizing the suspension;

FIG. 13 suspension containing homogenized particles shown in FIG. 12;

FIG. 14 shows textural properties measurement results for Theory Test Samples T #1A, T #1B, T #1C, and T #1D of Experiment III-1-B;

FIG. 15 shows textural properties measurement results for Theory Test samples T #2A, T #2B, T #2C, and T #2D in Experiment III-1-C;

FIG. 16A shows textural properties measurement results (positive area value) for Theory Test Samples T #1A and T #1B in Experiment III-1-D;

FIG. 16B shows texture properties measurement results (cohesiveness value) for Theory Test Samples T #1A and T #1B in Experiment III-1-D;

FIG. 17 shows texture properties measurement results for Theory Test Sample T #3A, 10% soy protein; and

FIG. 18 shows, on the left: cooked patty produced using blended commercial soy tofu as binder (Sample #New28) and on the right: cooked patty (Sample #New18).

Same reference numerals refer to similar structural details in all FIG.

### DETAILED DESCRIPTION

We were performing experiments in the kitchen-laboratory, to improve the structure of formed meat-replacement food product and observed, surprisingly, that one of the samples had a structure that was significantly different from the expected structure. It was much more elastic than the others, and resilient to bending. It was further much more easier to handle after shaping and before further processing such as cooking, frying, baking, steaming or grilling than the other samples. Then the inventors became curious.

After our surprising findings, we carried out a number of experiments with different samples. The purpose of the experiments was to find practical limits for the ranges, within which the invention is workable. We further performed experiments in a regular sized food manufacturing factory to produce formed meat-replacement food product in industrial scale and made observations. The effects of improvement by using treatments within the range of practical limits that we found in kitchen-laboratory were further verified as valid and advantageous for industrial-scale production of formed meat-replacement products that are meat imitates. The experiments are disclosed below.

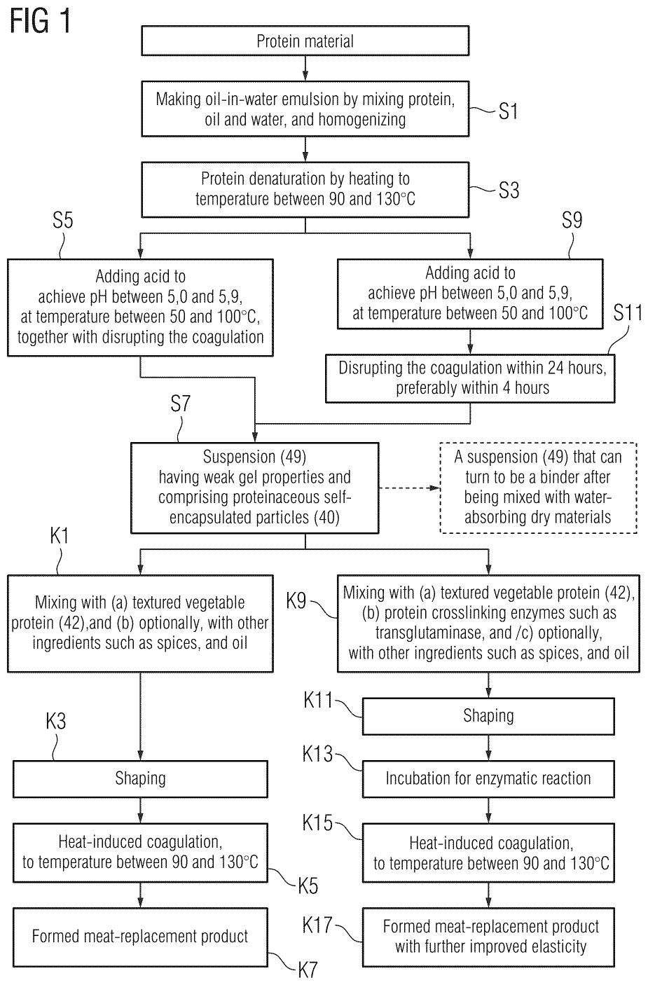

FIG. 1 shows manufacturing processes for manufacturing the proteinaceous binder ingredient and self-encapsulated proteinaceous particles in a suspension that can be used as a proteinaceous binder (steps S1-S11) that is particularly suitable for TVP's, especially TVP's manufactured with low-moisture texturization extrusion.

FIG. 1 shows also how the proteinaceous binder ingredient and self-encapsulated proteinaceous particles in a suspension can be used in the manufacture of formed meat-replacement products (steps K1-K17). The result of step S7 is the proteinaceous binder ingredient in a suspension or self-encapsulated proteinaceous particles in a suspension. They can be mixed (step K1) with (a) textured vegetable protein and (b), optionally, with other ingredients such as spices, and oil. Alternatively, the proteinaceous binder ingredient and self-encapsulated proteinaceous particles in a suspension can be mixed (step K9) with (a) textured vegetable protein, (b) protein crosslinking enzymes such as transglutaminase, and (c), optionally, with other ingredients such has spices and oil.

The other method steps in FIG. 1 are believed to be self-explanatory.

The temperatures indicated in FIG. 1 are such that the methods are workable. It is, however, possible that the methods can be made to work even beyond these temperatures.

The detailed processing steps and pathways are disclosed in the examples relating to Sample #1, Sample #12 (“Enzyme plus sample 1”), Sample #18, Sample #19 and Sample #20. For example, Sample #18 represents the process of adding acid to achieve a pH between 5.0 and 5.9, at temperature between 50 and 100° C. No crosslinking enzymes were added. Sample #19 and Sample #20 represent the process of coagulation-disrupting dispersion within 24 hours, preferably within 4 hours, no crosslinking enzymes were added. Sample #12 represents the process of having crosslinking enzymes added to the mixture of textured vegetable protein and the suspension composed of proteinaceous self-encapsulated particles.

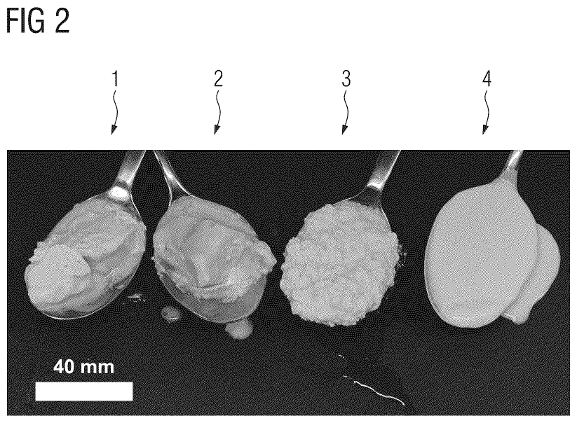

FIG. 2 illustrates the visually observed appearance of different mixtures after the acids were added to the cooked emulsions. Typical structure 1, 2 of curd-like gel made from non-disrupted-coagulation process, such as the non-disturbed-coagulum in example Sample #7 (“Coagulation not disrupted”). The structures 1, 2 of curd-like gels are clearly solid-like, self-standing, NOT-free-flowing, having clear sharp edges when it is cut by knife or spoon. The structure is quite close to tofu (soybean curd) or cheese appearance or texture. The structure 3 of plant-based proteinaceous binder ingredient of example Sample #1 that is manufactured with the respective methods involving the disrupted-coagulation process. The structure 3 of the proteinaceous binder ingredient is not a curd but is a suspension comprising, in addition to water or aqueous solution, also particles that have particle sizes larger than 0.101 mm but smaller than 10 mm, preferably between 0.3 mm and 5 mm, more preferably between 0.3 mm and 2 mm. There is clear syneresis water easily generated from this suspension. The syneresis water appear as clear phase between the individual solid particles and clusters.

If the coagulation is over-disrupted in step S5 or step S11, a homogenization takes place. This is illustrated with sample #8 (“Coagulation and over-disrupted by homogenization”) in the right-most test spoon. The photograph in FIG. 2 reveals that the structure 4 of coagulated and over-disrupted suspension is completely free-flowing, homogeneous, no remaining visible particles, close to appearance of a drinkable yogurt or mousse. There was no clear syneresis water generated in conditions like standing without centrifugation.

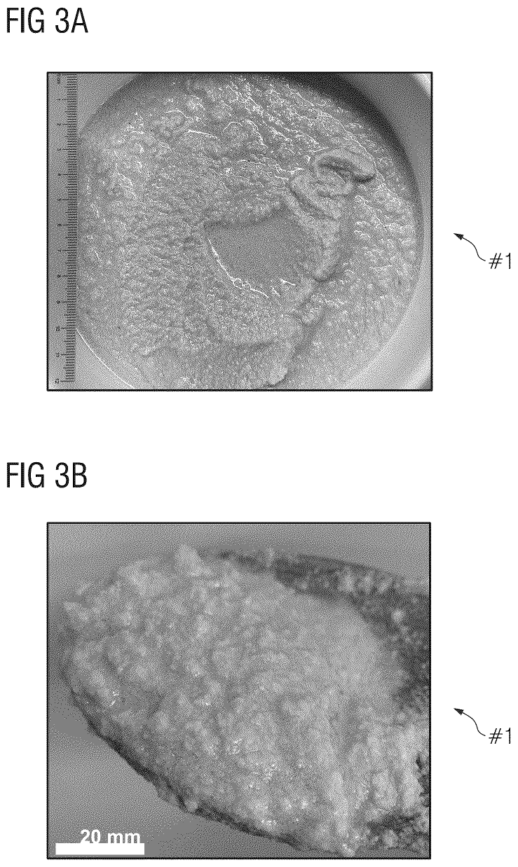

FIGS. 3A and 3B show the proteinaceous binder ingredient 49 of Sample #1 in a cup (FIG. 3A) and on a spoon (FIG. 3B). Both FIGS. 3A and 3B represent the typical suspension made from disrupted-coagulation process, which is the resulting product from step 2.c. of the example Sample #1 that is a disturbed-coagulum. The photographs it is visible that the disturbed-coagulum is not a curd but is composed of particles that have particle sizes larger than 0.101 mm and smaller than 10 mm, preferably between 0.3 mm and 5 mm, more preferably between 0.3 mm and 2 mm. There is clear syneresis water easily generated from this suspension. The longer the standing time of the suspension, the more syneresis water is generated.

In overall, as under conditions like standing without centrifugation, the syneresis may be less than 20%, preferably less than 10%, more preferably less than 5%. The syneresis water appears as clear phase between the individual solid particles and clusters. The suspension has certain properties of a free-flowing mixture. In other words, its structure 3 has flowability which is between a curd-like gel (structures 1, 2 in FIG. 2) and a liquid (structure 4 in FIG. 2).

### I Experiment

### Making of Plant Protein Based Patties: Comparing Different Binding Systems

Sample #1
Step 1 Textured plant-based proteins:
   Textured plant proteins, manufactured using a low moisture protein texturization extrusion method are puffy and in particulate form. The inventors prefer particulate form having a granule shape, such as the granules having a cylinder shape, approximately 5 mm diameter, 8 mm length. Other shapes, such as those listed in the next paragraph can also be used.
   Alternatively, pre-manufactured textured plant proteins manufactured using a low moisture protein texturization extrusion that are in particulate/granule/flake/chunk/thread form can be used. We have tested thread form like 1 mm diameter, 5 cm long or even longer
Step 2: A plant-based proteinaceous binder ingredient:
   Step 2.a. Weighing pea protein isolate, plant based liquid oil, and water, in a ratio of pea protein 12.5% (as-is weight), oil 5.0% (as-is weight), water 82.5% (as-is weight). Dispersing these ingredients with a blender, such as kitchen (preferably hand-held) immersion blender or kitchen blade grinder preferably equipped with rotating knife blades, or another homogenizer. The blended mixture is a protein-based oil-in-water emulsion (—hereinafter “Emulsion”).
   Step 2.b. Cooking the Emulsion with autoclave oven at 115° C. for 10 min, in order to increase the temperature of the emulsion to at least 95° C., preferably to at least 98° C., for more than 2 min, preferably more than 5 min. A cooked emulsion (—hereinafter “Cooked Emulsion”) is the result from this step.
   Step 2.c. After the Emulsion is cooked to make Cooked Emulsion, but before the Cooked Emulsion is chilled to below 70° C., adding vinegar (preferably white wine vinegar, having pH between 2.3 and 3.0, preferably between 2.5 and 2.8, most preferably between 2.6 and 2.7, for example, containing 4-8% acetic acid, preferably 6%) to the Cooked emulsion, the amount of the added vinegar is 3.3% of the cooked emulsion (as-is weight). Then the coagulation of mixture is disturbed preferably with a spatula or a whisk to break any formed large gel clusters (size of which is preferably at least 10 mm, such as between 10 mm and 200 mm) into smaller clusters or particles (size of which is larger than 0.101 mm and smaller than 10 mm, preferably between 0.3 mm and 5 mm, more preferably between 0.3 mm and 2 mm). The consistency of the mixture changes, resulting in formation of a disturbed-coagulum. The pH value of the dispersed-coagulate containing 3.3% vinegar was around 5.6.
The structure of the Coagulated-Emulsion-With-Disrupted-Coagulation was observed and shown in the structure 3 in FIG. 2, and in FIG. 3A, 3B.
Step 3. Combining textured plant proteins (cf. Step 1) with the plant-based proteinaceous binder ingredient (cf. Step 2 that is disturbed-coagulum), in a ratio of 125 g textured plant protein and 300 g dispersed coagulated emulsion (disturbed-coagulum).
Step 4. Mixing the combined textured plant proteins and plant-based proteinaceous binder ingredient, preferably with a planetary mixer. The inventors have used Hobart® (trademark of Premark FEG L.L.C.) N50 5-Quart model planetary mixer) equipped with “B” Flat Beater that provides mixing, kneading and shearing power on the mixture. Mixing for 1.5 min at the medium speed (124 RPM). As a result, the kneaded mixture (hereafter “Kneaded Mixture”) is the result from this step.
Step 5. Shaping the mixture (which preferably is Kneaded Mixture) from Step 4., by adding 150 g of the mixture into a porcelain baking cup having a cylinder inner shape, diameter of 90 mm, height/depth 55 mm. Evenly press the cup-contained kneaded mixture at its top surface, with power of 2.5 kg (24.5 newtons), which is equal to pressure of 3.9 kPa. A shaped patty (—hereafter “Shaped Patty”) is the result from this step.
Shaped Patty (shaped and uncooked patty) from Step 5 had a sufficient quality: it was resistant and elastic enough against pressing and bending and did not break apart into small crumbles or particles easily when pressed or bent. Therefore, it has good stability and intactness of shape after shaping and before cooking. Thus, the structure of the patty would be able to withstand shaking, moving, pressing and bending forces generally existing in normal food industrial manufacturing processes, and to minimize product losses during production. This is favourable and pivotal for product quality, production quality and capacity in industrial food processing, and less restrictive for the production process and operation. We found that a sufficient quality of shaped and uncooked patty, namely, resistant and elastic enough against pressing and bending, is a necessary-but-insufficient condition for the successful industrial production of plant-based meat-replacement food products.
Step 6. Processing (such as cooking, frying, steaming, grilling and/or baking, for example) the shaped patty (which preferably is Shaped Patty from Step 5) such that the emulsion undergoes further gelation under non-melting conditions for the proteins. The cooking can be carried out with autoclave oven at 115° C. for 10 min, in order to reach temperature of the emulsion to be above 95° C., preferably above 98° C., for more than 2 min, preferably more than 5 min. As a result, the cooked patty (—hereinafter “Cooked Patty”) is the result from this step.
Cooked Patty from Sample #1 had a sufficient quality: it was appealing in texture (chewiness, juiciness, elasticity, substantially mimicking mouthfeel of grilled beef patty), resistant enough against pressing and bending and did not break apart into small crumbles or particles easily when pressed or bent. Therefore, the good structural stability exhibited by the cooked patty would allow it to withstand possible shaking, moving, pressing and bending forces during transportation and storage.
We found that combined-conditions of condition (a) sufficient quality of shaped but uncooked patty; together with the condition (b) a sufficient quality of end-product (cooked patty), namely, having good mouthfeel that can substantially mimic formed meat-based products (e.g. grilled beef patty), can help to achieve successful industrial production of plant-based formed meat-replacement food products, not being limited to meat-replacement patties but in general for any kind of formed meat-replacement food products.
A more detailed description of the quality assessment of the samples and quantitative results is below in section “Instrumental Evaluation of the results of Experiment I”, in Tables 2, 3, and 4.

Sample #2 “Uncooked Emulsion”
Step 1. Textured plant-based proteins: Follow Step 1 for Sample #1.
Step 2. A plant-based proteinaceous binder ingredient: Follow Step 2 for Sample #1, however: omitting step 2.b. Step 2.c. reads:
Step 2.c. Adding vinegar (preferably white wine vinegar, having pH between 2.3 and 3.0, preferably 2.5 and 2.8, most preferably between 2.6 and 2.7) to the emulsion from Step 2.a. (which preferably is Emulsion), the amount of the added vinegar is 3.3% of the emulsion (as-is weight). Then dispersing the mixture with spatula or whisk to mix well. As a result, the dispersed acidified emulsion (—hereinafter “Dispersed Acidified Emulsion”) is the result from this step. The mixture did not coagulate so it was not possible to disrupt the coagulation.
Step 3. Combining the textured plant proteins (cf. Step 1) with plant-based proteinaceous binder ingredient (cf. Step 2c, Dispersed Acidified Emulsion), in a ratio of 125 g textured plant protein and 300 g dispersed acidified emulsion.
Step 4. Mixing the mixture of the textured plant proteins and Dispersed Acidified Emulsion with a planetary mixer (further details as in Step 4 for Sample #1). As a result, the Kneaded Mixture is the result from this step.
Step 5. Shaping—Follow Step 5 for Sample #1.
Step 6. Processing—Follow Step 6 for Sample #1.
Cooked Patty from Sample #2 had an insufficient quality: Omitting the heating, the mixture could not be converted to a weak gel and there was no coagulation. Acidification induced limited protein agglomeration that could not be converted into a weak gel. Thus, no treatment as disrupting of coagulation was possible. The cooked patty was too easy to break apart into small crumbles or particles by pressing or bending.
A more detailed description of the quality assessment of the samples and quantitative results also for these samples is below in section “Instrumental Evaluation of the results of Experiment I”, Table 2.

Sample #3: “8% pea protein in Emulsion”
Step 1. Textured plant-based proteins: Follow Step 1 for Sample #1.
Step 2. A plant-based proteinaceous binder ingredient: Follow Step 2 for Sample #1, except in Step 2.a. Weighing pea protein isolate, plant based liquid oil, and water, in a ratio of pea protein 8.0% (as-is weight), oil 5.0% (as-is weight), water 87% (as-is weight)
Step 3. Combining: Follow Step 3 for Sample #1.
Step 4. Mixing: Follow Step 4 for Sample #1.
Step 5. Shaping: Follow Step 5 for Sample #1.
Step 6. Processing: Follow Step 6 for Sample #1.

Cooked Patty from Sample #3 had a sufficient quality: it was appealing in texture (chewiness, juiciness, elasticity, substantially mimicking mouthfeel of grilled beef patty); and it was resistant enough against pressing and bending and did not break apart into small crumbles or particles easily when pressed or bent.

Samples #4A, #4B, #4C, #4D (“4%, 5%, 6%, or 7% Pea Protein in Emulsion”)
Step 1. Textured plant-based proteins: Follow Step 1 for Sample #1.
Step 2. A plant-based proteinaceous binder ingredient: Follow Step 2 for Sample #1, except in Step 2.a. Weighing pea protein isolate, plant based liquid oil, and water, in a few different ratio as recipes of
(A) pea protein 4.0% (as-is weight), oil 5.0% (as-is weight), water 91% (as-is weight)
(B) pea protein 5.0% (as-is weight), oil 5.0% (as-is weight), water 90% (as-is weight)
(C) pea protein 6.0% (as-is weight), oil 5.0% (as-is weight), water 89% (as-is weight)
(D) pea protein 7.0% (as-is weight), oil 5.0% (as-is weight), water 88% (as-is weight)
Step 3. Combining: Follow Step 3 for Sample #1.
Step 4. Mixing: Follow Step 4 for Sample #1.
Step 5. Shaping: Follow Step 5 for Sample #1.
Step 6. Processing: Follow Step 6 for Sample #1.
Cooked Patty from Samples #4A, #4B, #4C (pea protein content 4%-6%) had an insufficient quality: The emulsion had too little of plant-based proteinaceous ingredient so it did not coagulate properly, no proper coagulum could be formed, and in consequence no disrupting of the coagulation was possible. The cooked meat-replacement patty was too easy to break apart into small crumbles or particles by pressing or bending. Furthermore, the kneaded mixture from Step 4 was difficult to handle, it was too easy to separate apart between the textured proteins, and the mixture was difficult to be shaped or maintain the shape after shaping in Step 5.
Cooked Patty from Sample #4D (pea protein content 7%) had a sufficient quality: it was appealing in texture (chewiness, juiciness, elasticity, substantially mimicking mouthfeel of grilled beef patty); and it was resistant enough against pressing and bending and did not break apart into small crumbles or particles easily when pressed or bent.
A more detailed description of the quality assessment of the samples and quantitative results is in section “Instrumental Evaluation of the Results of Experiment I”, Table 3.

Sample #5 (“MORE VINEGAR”)
Step 1. Textured plant-based proteins: Follow Step 1 for Sample #1.
Step 2. A plant-based proteinaceous binder ingredient: Follow Step 2 for Sample #1, except in Step 2.c:
   Step 2.c. After said emulsion is cooked, before it is chilled to below 70° C., add vinegar (white wine vinegar, having pH between 2.3 and 3.0, preferably 2.5 and 2.8, most preferably between 2.6 and 2.7) to said cooked emulsion, the amount of the added vinegar is 6.6% by weight of the cooked emulsion (as-is weight). Then disrupt the coagulation preferably with spatula or whisk to break any formed large protein clusters (size of which is preferably at least 10 mm, such as between 10 mm and 200 mm) into clusters or particles between smaller size (size smaller than 10 mm, preferably smaller than 5 mm, more preferably smaller than 2 mm.) As a result, the disturbed-coagulum is made from this step. The pH value of the dispersed coagulated emulsion containing 6.6% by weight vinegar was around 5.1.
Step 3. Combining: Follow Step 3 for Sample #1.
Step 4. Mixing: Follow Step 4 for Sample #1.
Step 5. Shaping: Follow Step 5 for Sample #1.
Step 6. Processing: Follow Step 6 for Sample #1.
Cooked Patty from Sample #5 had a sufficient quality: it was appealing in texture (chewiness, juiciness, elasticity, substantially mimicking mouthfeel of grilled beef patty), resistant enough against pressing and bending and did not break apart into small crumbles or particles easily when pressed or bent.
A more detailed description of the quality assessment of the samples and quantitative results is in section “Instrumental Evaluation of the results of Experiment I”, table 4.

Sample #6 (“NO VINEGAR”)
Step 1. Textured plant-based proteins: Follow Step 1 for Sample #1.
Step 2. A plant-based proteinaceous binder ingredient: Follow Step 2 for Sample #1, except Step 2.c is omitted. The pH value of the cooked emulsion after step 2.b. i.e. containing no added acid was around 7.2.
Step 3: Combining the textured plant protein (cf. Step 1) with the cooked emulsion made in Step 2.b., in a ratio of 125 g textured plant protein and 300 g cooked emulsion.
Step 4. Mixing: Follow Step 4 for Sample #1.
Kneaded Mixture from Step 4 was difficult to handle, it was too easy to separate apart between the textured proteins, and the mixture was difficult to be shaped. In a normal industrial setting, it is very unlikely that a mixture with the characteristics displayed by this sample could be shaped.
Step 5. Shaping: Follow Step 5 for Sample #1.
Shaped Patty from Step 5 had an insufficient quality: it was too weak to keep the target shape from Shaping step (cf. Step 5) immediately after the Shaping process, and further too easy to break apart into small crumbles or particles by pressing or bending. Therefore, it has poor shape, or can hardly be preserved in good shape from commonly possible shaking, moving, pressing and bending forces being applied on the shaped products and generally existing in normal food industrial manufacturing processes.
Step 6. Processing: Follow Step 6 for Sample #1.
Cooked Patty from Sample #6 had an insufficient quality: In the absence of coagulation-causing treatment, the cooked emulsion showed no coagulation and thus it could not be made to disturbed-coagulum. It had unacceptable texture to be consumed as beef patty analogue product (poor chewiness, poor elasticity, doughy mouthfeel). The cooked patty was too easy to break apart into small crumbles or particles by pressing or bending.
A more detailed description of the quality assessment of the samples and quantitative results is in section “Instrumental Evaluation of the results of Experiment I”, Table 4.

Sample #7 (“Coagulation not Disrupted”, Also can be Referred to “Non-Disturbed Coagulum”)
Step 1. Textured plant-based proteins: Follow Step 1 for Sample #1.
Step 2. A plant-based proteinaceous binder ingredient: Follow Step 2 for Sample #1, except in step 2.c:
   Step 2.c. After said emulsion is cooked, before it is chilled to below 70° C., add vinegar (white wine vinegar, having pH between 2.3 and 3.0, preferably 2.5 and 2.8, most preferably between 2.6 and 2.7) to said cooked emulsion, the amount of the added vinegar is 3.3% of the cooked emulsion (as-is weight). Then gently mix the mixture with spatula without disrupting the coagulation i.e. without breaking the large protein clusters that are formed during the gelling (size of which is preferably at least 10 mm, such as between 10 mm and 200 mm). As a result, the coagulated emulsion (—hereinafter “Coagulated-Emulsion-Without-Disturbed-Coagulum”) is the result from this step.
The appearance of the Coagulated-Emulsion-Without-Disrupted-Coagulation was observed. It had the structure 1, 2 shown in FIG. 2.
Step 3. Combining: Follow Step 3 for Sample #1.
Step 4. Mixing: Follow Step 4 for Sample #1.
Kneaded Mixture from Step 4 was difficult to handle, it was too easy to separate apart between the textured proteins, and the mixture was difficult to be shaped.
Step 5. Shaping: Follow Step 5 for Sample #1.
Shaped Patty from Step 5 had an insufficient quality: it was too weak to keep the target shape from Shaping step (cf. Step 5) immediately after the Shaping process, and further too easy to break apart into small crumbles or particles by pressing or bending. Therefore, it has poor shape, or can hardly be preserved in good shape from commonly possible shaking, moving, pressing and bending forces being applied on the shaped products and generally existing in normal food industrial manufacturing processes.
Step 6. Processing: Follow Step 6 for Sample #1.
Cooked Patty from Sample #7 had an insufficient quality: After omitting the step of disrupting the coagulation. The resulted cooked patty had unacceptable texture to be consumed as beef patty analogue product (poor chewiness, poor elasticity, doughy mouthfeel). It was too easy to break apart into small crumbles or particles by pressing or bending.
A more detailed description of the quality assessment of the samples and quantitative results is in section “Instrumental Evaluation of the results of Experiment I”, Table 2.

Sample #8 (“Over-Disturbed Coagulum by Homogenization”)
Step 1. Textured plant-based proteins: Follow Step 1 for Sample #1.
Step 2. A plant-based proteinaceous binder ingredient: Follow Step 2 for Sample #1, except in step 2.c:
   Step 2.c. After said emulsion is cooked, before it is chilled to below 70° C., add vinegar (white wine vinegar, having pH between 2.3 and 3.0, preferably 2.5 and 2.8, most preferably between 2.6 and 2.7) to said cooked emulsion, the amount of the added vinegar is 3.3% of the cooked emulsion (as-is weight). Then substantially homogenize the mixture with a high-speed (rotating speed above 10000 rpm, revolutions-per-minute) Rotor Stator disperser homogenizer, or a fruit juice (or soup, or puree) blender equipped with rotating blade (rotating speed above 10000 rpm) for a time between 30 seconds and 60 seconds. Then the mixture became similar appearance, free-flowing flowability and gel strength as those soft-and-free-flowing yogurt, and/or drinkable yogurt.
   An over-disturbed coagulum (—hereinafter “Over-disturbed coagulum by homogenization”) is the result from this step.
The appearance of the Coagulated-and-Homogenized Emulsion was observed and shown in structure 4 in FIG. 2.
Step 3. Combining: Follow Step 3 for Sample #1.
Step 4. Mixing: Follow Step 4 for Sample #1.
Kneaded Mixture from Step 4 of Sample #8 was not as good as the Kneaded Mixture from step 4 of Sample #1.
Step 5. Shaping: Follow Step 5 for Sample #1.
Shaped Patty from Step 5 of Sample #8 was not as good as the Shaped Patty from Step 5 of Sample #1.
Step 6. Processing: Follow Step 6 for Sample #1.
Cooked Patty from Sample #8 had an insufficient quality: The resulted cooked patty had an unacceptable texture to be consumed as beef patty analogue product (poor chewiness, poor elasticity, doughy mouthfeel). It was too easy to break apart into small crumbles or particles by pressing or bending.

Sample #9 (“Replacing Vinegar by Same Dosage Lemon Juice”)
Step 1. Textured plant-based proteins: Follow Step 1 for Sample #1.
Step 2. A plant-based proteinaceous binder ingredient: Follow Step 2 for Sample #1, except in step 2.c.:
   Step 2.c. After said emulsion is cooked, before it is chilled to below 70° C., add lemon juice (having the same pH as the white wine vinegar, which is pH between 2.3 and 3.0, preferably 2.5 and 2.8, most preferably between 2.6 and 2.7) to said cooked emulsion, the amount of the added lemon juice is 3.3% of the cooked emulsion (as-is weight). Then disperse the mixture with spatula or whisk to break any formed large protein clusters (size of which is preferably at least 10 mm, such as between 10 mm and 200 mm) into clusters or particles between smaller size (size of which is smaller than 10 mm, preferably smaller than 5 mm, more preferably smaller than 2 mm.) As a result, the dispersed coagulated emulsion (“dispersed coagulated emulsion” hereafter) is made from this step. The pH of the dispersed coagulated emulsion containing 3.3% lemon juice was above 6.0 and below 6.2.
   It was not expected until the experiment that the pH of the emulsion acidified with lemon juice (above 6.0) was significantly higher than that acidified with vinegar (5.6), although the two acids had similar initial pH, and were used in the same dosage (as-is weight ratio of the cooked emulsion). The difference could be attributed to the different acid strength between the two types of acids. Acetic acid (main acid compound in vinegar) is a monobasic weak acid, whereas citric acid (main acid compound in lemon juice) is a weak tribasic acid that can behave as a buffer at 6.2 and below. Therefore, larger quantities of citric acid are required to reduce the pH when the pH is 6.2 or below. In next sample, a few different dosages of the vinegar and lemon juice were compared.
Step 3. Combining: Follow Step 3 for Sample #1.
Step 4. Mixing: Follow Step 4 for Sample #1.
Kneaded Mixture from Step 4 was difficult to handle, it was too easy to separate apart between the textured proteins, and the mixture was difficult to be shaped.
Step 5. Shaping: Follow Step 5 for Sample #1.
Shaped Patty from Step 5 had an insufficient quality: it was too weak to keep the target shape from Shaping step (cf. Step 5) immediately after the Shaping process, and further too easy to break apart into small crumbles or particles by pressing or bending. Therefore, it has poor shape, or can hardly be preserved in good shape from commonly possible shaking, moving, pressing and bending forces being applied on the shaped products and generally existing in normal food industrial manufacturing processes.
Step 6. Processing: Follow Step 6 for Sample #1.
Cooked Patty from step 6, Sample #9 had insufficient quality: The resulted cooked patty had unacceptable texture to be consumed as beef patty analogue product (poor chewiness, poor elasticity, doughy mouthfeel). it was too easy to break apart into small crumbles or particles by pressing or bending.

Samples #10A to #10H) (“Different Dosages of Vinegar and Lemon Juice”)
Step 1. Textured plant-based proteins: Follow Step 1 for Sample #1.
Step 2. A plant-based proteinaceous binder ingredient: Follow Step 2 for Sample #1, except in step 2.a. and step 2.c.:
   Step 2.a. Weighing pea protein isolate, plant based liquid oil, and water, in a ratio as recipes (a) pea protein 12.5% (as-is weight), oil 5.0% (as-is weight), water 82.5% (as-is weight); (b) pea protein 10% (as-is weight), oil 5.0% (as-is weight), water 85% (as-is weight). Blending these ingredients with a disperser, such as kitchen (preferably hand-held) immersion blender or kitchen blade grinder preferably equipped with rotating knife blades, or another homogenizer. The blended mixture is a protein-based oil-in-water emulsion (—hereinafter “Emulsion”).
   Step 2.c. After said emulsion is cooked, before it is chilled to below 70° C., add acids (vinegar or lemon juice) to said cooked emulsion, the amount of the added acid 1.6%-13.2% of the cooked emulsion (as-is weight). Then disperse the mixture with spatula or whisk to break any formed large protein clusters (size of which is preferably at least 10 mm, such as between 10 mm and 200 mm) into clusters or particles having smaller size (size of which is smaller than 10 mm, preferably smaller than 5 mm, more preferably smaller than 2 mm but larger than 0.101 mm.) As a result, the dispersed acidified emulsion (“dispersed acidified emulsion” hereafter) is made from this step. The pH of the acidified emulsions are listed in Table 1.
Step 3. Combining: Follow Step 3 for Sample #1.
Step 4. Mixing: Follow Step 4 for Sample #1.
Kneaded Mixture results are listed in Table 1.
Step 5. Shaping: Follow Step 5 for Sample #1.
Shaped Patty from Step 5 quality results were listed in Table 1.
Step 6. Processing: Follow Step 6 for Sample #1.
Cooked Patty from Step 6 quality results were listed in Table 1.

TABLE 1
Quality results and pH value of shaped and cooked patties prepared in Samples #10A to #10H

| | Pea | Acid type and | | Results of formed product before | Results of end product after |
|---|---|---|---|---|---|
| #10 | protein * | dosage * | pH value ** | cooking | cooking |
| A | 12.5% | Vinegar, 1.6% | 6.26 | Bad, easy to break, holding force | Bad, doughy, poor cohesiveness |
| | | | | is poor | |
| B | 12.5% | Vinegar, 3.3% | 5.64 | Good, intact, easy to handle | Good, cohesive, chewy, elastic |
| C | 12.5% | Lemon juice, 3.3% | 6.04 | Bad, easy to break, holding force | Bad, doughy, break by bending |
| | | | | is poor | |
| D | 12.5% | Lemon juice, 4.4% | 5.89 | Good, intact, easy to handle | Good, cohesive, chewy, elastic |
| E | 12.5% | Vinegar, 6.6% | 5.10 | Good, intact, easy to handle | OK, cohesive, chewy, elastic |
| F | 10% | Vinegar, 1.3% | 6.40 | Bad, easy to break, holding force | Bad, doughy, break by bending |
| | | | | is poor | |
| G | 10% | Vinegar, 2.5% | 5.78 | Good, intact, easy to handle | Good, cohesive, chewy, elastic |
| H | 10% | Vinegar, 13.2% | 4.59 | OK, intact | Bad, doughy, poor cohesiveness |

* The “dosage” was referring to the as-is weight ratio of the acid of the cooked emulsion.
** The “pH value” was referring to the pH of the disturbed-coagulum containing either white wine vinegar or lemon juice.
Additional Note 1:
The measured pH of the emulsion containing 12.5% pea protein was approximately 7.2. The measured pH of the vinegar and lemon juice used in the experiment was, respectively, approximately 2.7 and approximately 2.6.

As can be seen from results in Table 1, the pH value of the mixture (acidified cooked emulsion) is fundamentally critical for the quality of the shaped but not processed (such as cooked, fried, grilled, baked) intermediate product, and for the quality of the formed meat-replacement food product (end product). The choice of dosage of acid addition is dependent on the nature of the acid (mono-, di- and triprotic), its strength (pKa), and the concentration of the selected acid, in the selected substance (for example, vinegar may contain from 4-8% acetic acid), and should be confirmed with the pH value of the acidified cooked emulsion. It is necessary and beneficial to select the dosage and type of acid so that the acidified cooked emulsion will achieve a pH value between 5.0 and 5.9, which was required to result in good texture and mouth-feel properties of the product (cohesive, chewy and elastic).

When the pH value was near 4.59 (sample #10H) as being close to the isoelectric point (also known as “pI value”) of the pea protein, the quality of the end product (cooked patty) was poor, for example, its mouthfeel was doughy, and its elasticity (cohesiveness) was insufficient.

When there was too little acid added (sample #10A, sample #10C and sample #10F), the pH value (6.04-6.40) was close to neutral, and the quality of both the intermediate product (shaped but not cooked patty) and the end product (cooked patty) was poor.

Samples #11A, #11B (“Insufficient Mixing/Kneading on Mixture of Textured Protein and Binder”)
Step 1. Textured plant-based proteins: Follow Step 1 for Sample #1.
Step 2. A plant-based proteinaceous binder ingredient: Follow Step 2 for Sample #1
Step 3. Combining: Follow Step 3 for Sample #1.
Step 4. Mixing: with a few different methods that are weaker than Step 4 for Sample #1.
   Sample #11A: Only hand mixing to achieve an even distribution between the textured plant-based proteins (cf. Step 1) and the plant-based proteinaceous binder ingredient (cf. Step 2).
   Sample #11B: First hand mixing to achieve an even distribution between the textured plant-based proteins (cf. Step 1) and the plant-based proteinaceous binder ingredient (cf. Step 2). Then mix the mixture with of the textured plant protein and dispersed coagulated emulsion with a planetary mixer as for Sample #1, Mixing for 0.5 min at the medium level of speed (124 RPM, round per minute). As a result, the 0.5 min kneaded mixture (“0.5 min kneaded mixture” hereafter) is made from this step.
Kneaded Mixture Sample of #11A from Step 4 was difficult to handle, it was too easy to separate apart between the textured proteins, and the mixture was difficult to be shaped. Hand mixing of the textured plant-based proteins and disturbed coagulum allowed an even distribution of the components. However, this method was clearly inadequate for generating a cohesive raw mass under normal shaping conditions. Despite extended hand mixing, the cohesiveness of the mix did not improve.
Step 5. Shaping: Follow Step 5 for Sample #1.

Shaped Patty of Sample #11A from Step 5 had an insufficient quality: it was too weak to keep the target shape from Shaping step (cf. Step 5) immediately after the Shaping process, and further too easy to break apart into small crumbles or particles by pressing or bending. Therefore, it has poor shape, or can hardly be preserved in good shape from commonly possible shaking, moving, pressing and bending forces being applied on the shaped products and generally existing in normal food industrial manufacturing processes.

For Kneaded Mixture Sample #11B, mixing for 0.5 min resulted in a very weak structure that could barely hold together its poorly shaped structure. Therefore, a patty produced in this manner is easy to break apart and would require extreme care during manufacturing. On the industrial level a raw patty should be able to withstand several stresses such as shaking, moving, pressing, etc. Therefore, this type of raw patty is unsuitable for large scale manufacturing as it would result in a very large percentage of rework/food waste.
Step 6. Processing: Follow Step 6 for Sample #1.
Cooked Patties from Samples #11A and #11B had insufficient quality: The resulted cooked patty had unacceptable texture to be consumed as beef patty analogue product (poor chewiness, poor elasticity, doughy mouthfeel). It was too easy to break apart into small crumbles or particles by pressing or bending.

Sample #12 (Enzyme Plus Sample #1)
Step 1. Textured plant-based proteins: Follow Step 1 for Sample #1.
Step 2. A plant-based proteinaceous binder ingredient: Follow Step 2 for Sample #1
Step 3. Combining the textured plant protein (cf. Step 1) with the plant-based proteinaceous binder ingredient (cf. Step 2 that preferably is a Disturbed-Coagulum, most preferably manufactured as disclosed in Step 2.c), and protein crosslinking (binding) enzyme, such as transglutaminase, in a ratio of 125 g textured plant protein and 300 g Disturbed-Coagulum and enzyme 4.3 g.
Step 4. Mixing: Follow Step 4 for Sample #1. The temperature of environment of carrying out the mixing is below 40° C., preferably between 1 and 25° C., to avoid that the enzyme reacts too fast and earlier than the shaping step. Otherwise, it will be difficult or impossible to have the meat-replacement patty shaped properly.
Step 5. Shaping the kneaded mixture from Step 4, by packing the mixture in a vacuum bag with a suitable vacuum level, such as 98%, then flatten the mixture to thickness of 15 mm, then take the sample out from vacuum, so the mixture is kept under vacuum for less than 10 min. This process is to mimic typical food industrial shaping machine with which the meat-replacement product is shaped, such as plate mould forming machine (such as Forming Machine AFM from supplier Alco-food-machines GmbH & Co.), and vacuum assisted stuffing and forming machine. As a result, the shaped patty is made from this step. The time between the kneading and the forming/shaping steps should be less than 4 hour, preferably less than 1 hour, more preferably less than 30 min. The temperature of storing, buffering and transferring environment of between the kneading and the forming/shaping steps is below 40° C., preferably between 1 and 35° C., more preferably between 15 and 25° C. to avoid that the enzyme reacts too fast and earlier than the forming step, which will make the shaping difficult or impossible.
Shaped Patty of Sample #12 from Step 5 had a sufficient quality: it was resistant enough against pressing and bending and did not break apart into small crumbles or particles easily when pressed or bent. Therefore, it has good stability and intactness of shape after shaping and before further processes (incubation and cooking), despite commonly possible shaking, moving, pressing and bending forces being applied on the shaped products and generally existing in normal food industrial manufacturing processes.
Step 6. Incubate the shaped patty from Step 5.
Containing enzyme to temperature above 30° C., preferably between 40 and 50° C., for a time between 30 min and 4 hour, preferably between 1 hour and 2 hour. Alternatively, incubate the shaped patty at temperature between 1° C. and 6° C., for a time between 4 hour and 36 hour, preferably between 8 hour and 16 hour.
Step 7. Processing (such as cooking, frying, grilling, steaming and/or baking, for example) the shaped and incubated patty such that the emulsion undergoes further gelation under non-melting conditions for the proteins. The cooking can be carried out with autoclave oven at 115° C. for 10 min, in order to reach temperature of the emulsion to be above 95° C., preferably above 98° C., for more than 2 min, preferably more than 5 min. As a result, a cooked patty is the result of this step. Step 7. can be a direct continuation of the step 6, by, for example, after heating the shaped patty in an autoclave oven to temperature above 30° C., preferably between 40 and 50° C., for a time between 30 min and 4 hour, and then directly further increase the temperature to 115° C.
Cooked Patty from Sample #12 had a sufficient quality: it was appealing in texture (chewiness, juiciness, elasticity, substantially mimicking mouthfeel of grilled beef patty), resistant enough against pressing and bending and did not break apart into small crumbles or particles easily when pressed or bent.

### Instrumental Evaluation of the Results of Experiment I

Note: one should only compare data within the same table. The different tables are not comparable with each other.

For the Hardness, Springingness, Cohensiveness, Chewiness, Surface Area and Resilience measurement, we measured the resistance forces of the samples during a compression test. The measurements were carried out so that the TA.XTPlus Texture Analyzer was equipped with a 294.2 N (30 kg) load cell (detector sensor) and different detection probes.

The measurement program was adopted from a standard TPA measurement protocol (Citation from [3] the manual of the measurement equipment “Texture profile analysis (TPA) is an objective method of sensory analysis. TPA is based on the recognition of texture as a multi-parameter attribute. For research purposes, a texture profile in terms of several parameters determined on a small homogeneous sample may be desirable. The test consists of compressing a bite-size piece of food two times in a reciprocating motion that imitates the action of the jaw and extracting from the resulting force-time curve a number of textural parameters that correlate well with sensory evaluation of those parameters. The mechanical textural characteristics of foods that govern, to a large extent, the selection of a rheological procedure and instrument can be divided into the primary parameters of hardness, cohesiveness, springiness, and adhesiveness, and into the secondary (or derived) parameters of fracturability (brittleness), chewiness and gumminess.”

The downward speed before the probe touching the sample was 5 mm/s (pre-test speed). The speed of compression when the blade touched the sample was 5 mm/second (test speed) and compression went to a compression depth until 65% of the height of the sample was reached. Then the probe withdraw (move upward) with speed (post-test speed) 5 mm/second. There was a “trigger force” setting, which was set as 5 g in this study. The waiting time between the first and the second compression was 3 s. The Hardness, cohesiveness, springiness, adhesiveness, chewiness, gumminess and resilience values are calculated by the software of the measurement equipment. (Citation from [4]): “Hardness was expressed as the maximum force of the 1st compression. The Hardness value is the peak force that occurs during the first compression. The hardness need not occur at the point of deepest compression, although it typically does for most products. Cohesiveness was the area of work during the second compression divided by the area of work during the first compression. Cohesiveness is how well the product withstands a second deformation relative to its resistance under the first deformation. Springiness was expressed as a ratio or percentage of a product's original height. Springiness was how well a product physically springs back after it has been deformed during the first compression and has been allowed to wait for the target wait time between strokes. The springback is measured at the down-stroke of the second compression. Chewiness was calculated as Hardness*Cohesiveness*Springiness. Chewiness was only applied to solid products and is calculated as Gumminess*Springiness. Resilience was calculated by dividing the upstroke energy of the first compression by the downstroke energy of the first compression. Resilience was how well a product “fights to regain its original height”. Resilience was measured on the withdrawal of the first penetration, before the waiting period is started. Resilience can be measured with a single compression; however, the withdrawal speed must be the same as the compression speed.”

The shape, size and placement of the analysed sample and the analysis configuration geometry were described in details near each section of results below.

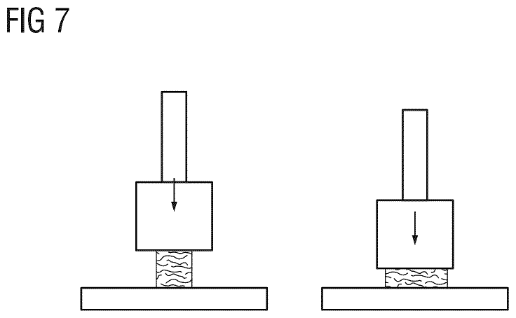

Cooked patty samples #1, #2, and #7 were evaluated for hardness, cohesiveness, chewiness, and resilience in a TPA assay at room temperature, using the 36 mm diameter cylinder shaped detection probe model “P/36R”, 36 mm Radius Edge Cylinder probe—Aluminium—AACC Standard probe for Bread firmness, from Stable Micro Systems, Ltd. The illustration of the compressing system including the detection probe, base platform and sample, as shown in FIG. 7. A ground beef patty (20% fat) was used as a reference. Results are shown on Table 2.

TABLE 2
Texture analysis, compressing the cooked patty (a
cubic sample 15 mm each dimension) taken out from the cup by
a texture analyser using parallel plate compression system
(equipped with a 36 mm diameter cylinder shaped detection
probe and a flat platform as the basis of the sample.)

| | | Hard- | | | |
|---|---|---|---|---|---|
| | | ness | Cohesive- | Chewi- | Resil- |
| | Main difference | (kg) | ness | ness | ience |
| Sample #1 | Emulsion was cooked | 4.1 | 46% | 1211 | 16% |
| | before adding vinegar, | | | | |
| | disrupted coagulation | | | | |
| Sample #2 | Emulsion was not cooked | 3.3 | 39% | 663 | 13% |
| | before adding vinegar | | | | |
| Sample #7 | Emulsion was cooked | 8.9 | 36% | 2343 | 11% |
| | before adding vinegar, | | | | |
| | non-disrupted coagulation | | | | |
| Reference | Grill cooked beef patty cut | 5.0 | 57% | 2361 | 23% |
| #Beef | into the same shape | | | | |

TA.XTPlus Texture Analyzer (supplier Stable Micro Systems) was used to conduct the texture analysis in this and all following experiments. The detection probe was model “P/36R”, 36 mm Radius Edge Cylinder probe, supplier Stable Micro Systems. The illustration of the compressing system including the detection probe, base platform and sample, as show in FIG. 7.

Results in Table 2 show that: When the samples were analysed in shape of cubic with 15 mm each dimension, the reference sample #beef has a relatively high cohesiveness (around 57%), chewiness (2361) and resilience (23%), while not necessarily high hardness (5.0 kg). That matches the desirable perceptions from consumers about beef patty texture and mouthfeel, which should not be hard or stiff or rubbery, but should be to certain extent elastic, cohesive and chewable. Sample #1 had cohesiveness (46%) and resilience (16%) values closest to those of the beef patty (Sample Reference #Beef), compared to Sample #2 and Sample #7. Sample #2 had a lower value for all aspects analyzed, which matched its mouthfeel and texture properties as weak, soft, doughy and easy to break-apart, and not desirable as meat-replacement product. The poor quality is caused by the lack of cooking step on the emulsion, and therefore, lack of protein denaturation and proper conditions for coagulation. Sample #7 had worse cohesiveness and resilience properties than Sample #1, though it had higher hardness and chewiness than Sample #1. This showed and matched with the sensorial observation of insufficient texture and mouth-feel quality of Sample #7, which was lack of elastic mouthfeel, lack of integrity against compressing or biting, and easy to break apart.

Further, the high hardness of Sample #7 beside the other textural parameters could make the product mouth-feel dry, stiff and hard. The poor quality of Sample #7 was attributed to its proteinaceous binder ingredient, which was not made by disrupted-coagulation but normal coagulation (so called “non-disrupted-coagulation”).

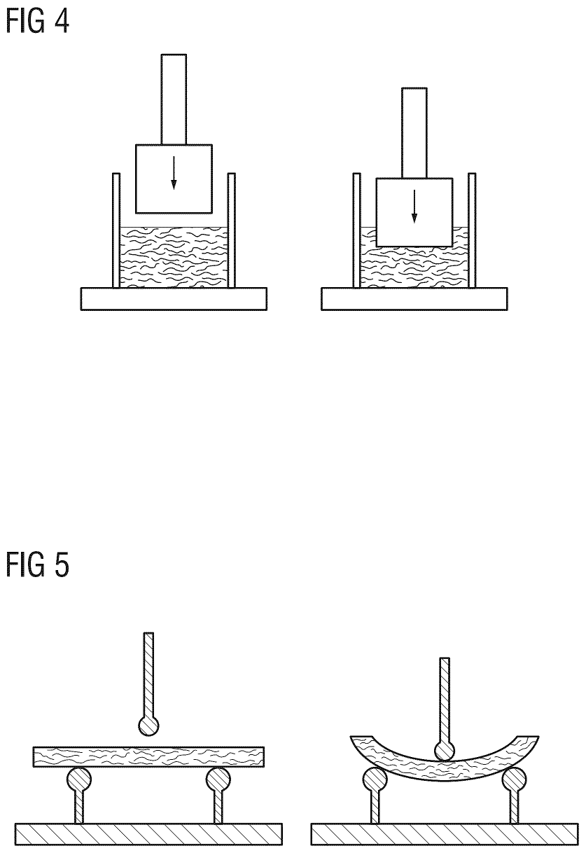

The minimum protein concentration in the preparation of the disturbed-coagulum to be able to obtain an uncooked and cooked shaped plant-based product of acceptable quality was investigated. Quality of the cooked samples was assessed TPA assay performed at room temperature, using the aforementioned “P/36R” probe. The illustration of the compressing system including the detection probe, base cup-and-platform and sample, as shown in FIG. 4. Results obtained from the analysis are in Table 3.

TABLE 3
Texture analysis, compressing the cooked patty in
the cup by a texture analyser equipped with a cylinder (36
mm diameter) as detection probe

| | | Hard- | |
|---|---|---|---|
| | | ness | Chewi- |
| | Main difference | (kg) | ness |
| Sample #4A | Step 2.a. pea protein 4.0% in emulsion | 8.4 | 4357 |
| Sample #4C | Step 2.a. pea protein 6.0% in emulsion | 10.4 | 3077 |
| Sample #4D | Step 2.a. pea protein 7.0% in emulsion | 19.5 | 5038 |
| Sample #1 | Step 2.a. pea protein 12.5% in emulsion | 16.3 | 5812 |
| Reference | Cooked patty made of minced beef | 14.0 | 5220 |
| #minced beef | meat | | |

* Note:
Reference #Minced Beef. Prepared as: Filling 150 g minced beef meat (20% fat) in a Porcelain baking cup having a cylinder inner shape, diameter of 90 mm, height/depth 55 mm. Evenly pressing the cup-contained minced beef meat at its top surface, with power of 2.5 kg (24.5 newtons). Then cooking it with autoclave oven at 115° C. for 10 min.
** Note 2:
Typical texture profile analysis (TPA) was conducted on the compression tests. Before the analysis, the samples were tempered to room temperature around 20° C.

FIG. 4 illustrates the compressing system including the detection probe, base cup-and-platform and sample inside the cup. The detection probe is pressed into the cup holding the sample. The measurement condition and geometry of samples for the results shown in Table 3 (sample contained in a cup) were very different from those shown in Table 2 for a cubic sample having a size of 15 mm in each dimension. Therefore, the hardness and chewiness values in these two tables are not directly comparable. In the analysis using cup as a container, the hardness and chewiness values are relatively more important. Products with too low hardness and chewiness values were found having poor integrity, and mash-like and dough-like (doughy) mouthfeel or texture. When protein content in emulsion were below 6.1%, such as 4.0%-6.0% (Sample #4A and Sample #4C), the products had hardness lower than 12 kg, clearly lower than that of the cooked minced beef meat sample (14 kg), and largely lower than that of the samples having protein content larger than 7% (Sample #4D and Sample #1). Similar trend was found in the chewiness values of these samples, too.

The role of vinegar and different vinegar doses on the quality of the cooked patty was investigated. Quality of the cooked samples was assessed TPA assay performed at room temperature, using the aforementioned “P/36R” probe. The compressing system including the detection probe, base cup-and-platform and sample was as shown in FIG. 4. Results obtained from the analysis are shown in Table 4.

TABLE 4
Texture analysis, compressing the cooked patty in
the cup by a texture analyser, samples were prepared
slightly differently (Note: The B samples were made smaller:
only 80 g of the kneaded mixture was in each cup.)

| | Hardness (kg) | Chewiness |
|---|---|---|
| Sample #6B (80 g) No vinegar | 12.4 | 5821 |
| Sample #1B (80 g) 3.3% vinegar | 18.3 | 8551 |
| Sample #5B (80 g) 6.6% vinegar | 16.2 | 6723 |

The compressing system including the detection probe, base cup-and-platform and sample was as shown in FIG. 4. The texture analysis set-up for results shown in Table 4 were different from that in Table 3, because the sample volume was smaller, though the other parameters are the same. Therefore, the hardness and chewiness values in these two tables were not directly comparable. Table 4 shows that the vinegar dosage 3.3% resulted in a firmer texture of the product (Sample #1B) than vinegar dosage 0% (Sample #6B) and 6.6% (Sample #5B). Sample #5B was closer to Sample #1B, and sensorial evaluation results showed that these two samples had sufficient end-product quality (texture and mouthfeel being elastic, cohesive and chewy). On the other hand, the Sample #6B had much lowered hardness and chewiness values than Sample #1B, which was in agreement with the sensorial evaluation results that the Sample #6B had insufficient quality (texture and mouthfeel being doughy, soft, easy to break).

### II Experiment

### II-1. Making of Plant Protein Based Patties: Comparing Different Particles and Textured Proteins

Samples #13, #14, #15 and #16
Step 1. Prepare textured plant protein or particles:
   FOR Sample #13: Textured plant protein (also known as “textured vegetable protein”, or as “TVP”), made by low moisture protein texturization extrusion, puffy, porous, having granule shape, having continuous proteinaceous fibrous matrix structure that is substantially linearly oriented (cylinder shape, approximately 5 mm diameter, 8 mm length). (Composition: 65% mixture of pea and faba bean protein isolate and concentrate, 35% oat ingredients such as bran, flours and protein). Named as “Granule” hereafter. (Bulk density: 100-260 g/L, preferably 160-240 g/L; 10-min water hydration capacity, above 280%, more frequently between 330% and 420%, which is a ratio between the weight of water that can be absorbed by the textured plant protein to the weight of the textured plant protein after the textured plant protein is immersed in an excessive amount of water for ten minutes, the water that is not absorbed by the textured plant protein is then drained by sieving). The calculation of 10-min water hydration capacity can be made according to the equation of:

“10-min water hydration capacity=100%*(weight of textured plant protein after 10 min water hydration−weight of the textured plant protein before hydration)/weight of the textured plant protein before hydration”
FOR Sample #14: Extrusion cooked protein product, which is dense, flake shape (approximately 5 mm diameter, 8 mm length, 8 mm), substantially lack porosity, not sponge-like, insufficient (poor) structural and textural stability for withstanding hydration in cooking. (Composition: 65% mixture of pea and faba bean protein isolate and concentrate, 35% oat ingredients such as bran, flours and protein). Named as “Dense Flake” hereafter. (Bulk density: 270-350 g/L, more frequently 280-300 g/L; 10-min water hydration capacity, 230%-250%)
FOR Sample #15: Textured plant protein, commercial, (Composition: 100% soy protein concentrate) made by low moisture protein texturization extrusion, which is puffy, granule shape (cylinder shape, approximately 5 mm diameter, 8 mm length; bulk density: 170-180 g/L; 10-min water hydration capacity, around 350%). Named as “Soy puffy” hereafter.
FOR Sample #16: Steel cut oat particles. Named as “Steel cut oat” hereafter. (Bulk density: 350-400 g/L; 10-min water hydration capacity, 30%-45%. Not porous at all.)
Step 2. A plant-based proteinaceous ingredient which is a disturbed-coagulum: Follow Step 2 for Sample #1.
Step 3. Combining: Follow Step 3 for Sample #1.
Step 4. Mixing: Follow step 4 for Sample #1.
Step 5. Shaping: Follow step 5 for Sample #1.
Step 6. Processing: Follow step 6 Sample #1.

### II-2. Stability of Shape of Raw Shaped (not Cooked) Plant Protein Based Patties Analysed by Bending Tests: Comparing Different Binder Ingredients (e.g. Different Amount of Oil in Emulsion; Different Amount of Protein in Emulsion; Different Amount of Acid; with and without Cooking of Emulsion; Non-Disrupted Coagulation and Disrupted-Coagulation; with and without Homogenization after Coagulation)

### And Preparing Samples for II-4

Samples #1C, #17 to #28

Sample Preparation:
Step 1. Prepare textured plant protein in the same way as in Step 1 of Sample #1.
Step 2. Prepare proteinaceous matrix (“binder”):
   Step 2.a. Weighing pea protein isolate, plant based liquid oil, and water, in different ratios, for example, for Sample #17, the ratio was pea protein 10% (as-is weight), oil 5.0% (as-is weight), water 85% (as-is weight); otherwise, for some other samples, in other ratios as specified in Table 6. Disperse these ingredients with a disperser, such as kitchen hand-held immersion blender, equipped with rotating knife blades, or other homogenizators. The blended mixture is a protein-based oil-in-water emulsion (“emulsion” hereafter).
   Step 2.b. Cook said emulsion with autoclave oven at 115° C. for 10 min, in order to reach temperature of the emulsion to be above 95° C., preferably above 98° C., for more than 2 min, preferably more than 5 min. As a result, the cooked emulsion (“cooked Emulsion” hereafter) is made from this step. Otherwise, for some other samples, such as Sample #27, do not apply this cooking step. The choice of cooking is specified in the Table 6.
   Step 2.c. After said emulsion is cooked (for samples require cooking step), before it is chilled to below 70° C., add vinegar to said cooked emulsion, the amount of the added vinegar is, for example, for Sample #18, 3.3% of the cooked emulsion (as-is weight); otherwise, for some other samples, in other amounts as specified in Table 6. Then conduct Disrupted-Coagulation treatment, which was a cluster-disrupting stirring treatment, by dispersing the mixture with spatula or whisk to break any formed large protein clusters (size no less than 10 mm, preferably between 10 mm and 200 mm) into clusters or particles between smaller size (size smaller than 10 mm, preferably smaller than 5 mm, more preferably smaller than 2 mm.) As a result, the disturbed coagulum (“Disturbed-coagulum” hereafter) is made from this step.
   Nevertheless, for some other samples, such as Sample #21 and Sample #23, the Disrupted-Coagulation treatment was replaced by Non-Disrupted-coagulation (by gently mixing the mixture with spatula without disrupting the coagulation i.e. without breaking the formed large protein clusters during the gelling, Similar as the acid addition method in Step 2.c. of Sample #7), OR Over-Disrupted-Coagulation (by homogenization using a fruit juice blender equipped with rotating blade, rotating speed above 10000 rpm, for a time between 30 seconds and 60 seconds. Similar as the homogenization treatment in Step 2.c. of Sample #8). The choice of Non-Disrupted-coagulation, OR Over-Disrupted-Coagulation for each sample was specified in Table 6.
Step 3. For Samples #17 to #27, Combine the textured plant protein made in Step 1., with the dispersed coagulated emulsion made in Step 2.c., in a ratio of 150 g textured plant protein and 300 g dispersed coagulated emulsion, and 10 g rapeseed oil.
Otherwise, for Sample #28, Combine the textured plant protein made in Step 1., with blended commercial soy tofu (soybean curd) in a ratio of 150 g textured plant protein and 300 g blended commercial soy tofu and 10 g rapeseed oil.
Further differently, for Samples #1T (the same as Sample #1 but with transglutaminase enzyme), #18T, #21T, #22T, #30T, transglutaminase enzyme was added, the exact dosage information is listed in the table.
Step 4. Mix the mixture of the textured plant protein and dispersed coagulated emulsion with a planetary mixer for 1.5 min, in the same way as in Step 4 of Sample #1. As a result, the kneaded mixture (“kneaded mixture” hereafter) is made from this step.
Step 5. Shape the kneaded mixture from Step 4., by packing the mixture in a vacuum bag with vacuum level 98%, then flatten the mixture to thickness of 15 mm, then take the sample out from vacuum, so the mixture is kept under vacuum for less than 10 min. This process is to mimic typical food industrial forming or shaping machine, such as plate mold forming machine, and vacuum assisted stuffing and forming machine. As a result, the shaped patty (“shaped patty” hereafter) is made from this step.
Differently, for samples #1T, #18T, #21T, #22T, #30T, the shaped patties from Step 5. were incubated in an oven at 50° C. for 1 hour, before they were proceeded to Step 7 (cooking).
Step 6. Evaluate some of the shaped patty from Step 5. with the three position compression bending measurement. Since in measurement the tested materials are deformed, samples are discarded after testing. The tested sample materials were not proceeded to further Step 7. The samples that were NOT evaluated or which did not deform in Step 6, were proceeded to Step 7 (cooking)
Step 7. Cook the shaped patty from Step 5, with autoclave oven at 115° C. for 10 min, in order to reach temperature of the emulsion to be above 95° C., preferably above 98° C., for more than 2 min, preferably more than 5 min. As a result, the cooked patty (“cooked patty” hereafter) is made from this step.
Preparation of Sample #1C: Step 1, Step 2, Step 3 and Step 4 were following Step 1, Step 2, Step 3 and Step 4 of Sample #1; and then Step 5, Step 6 and Step 7 were following Step 5, Step 6 and Step 7 of Sample #18C.
The produced samples include: (group 1) Sample #1C, #18C, #20C, #21C, #23C, #27C, which were cooked versions of Sample #1, #18, #20, #21, #23, and #27 that have the same composition before the cooking step; and (group 2) samples #1T, #18T, #21T, #22T, #30T, which contained transglutaminase enzyme and were cooked.
Detail description of the quality assessment of the uncooked samples and quantitative results are shown in the evaluation of the results of experiment II section, Table 6. Results for the cooked samples are shown in Table 7 and Table 8.

### Texture Analysis Method

### Three Position Compression Bending Test

FIG. 5 illustrates the three-position compression bending test system we used to evaluate the samples. There is a rack on a base platform, which has two linear guides having a rounded upper edge (approximately 3 mm diameter). The linear guides make up two base points having a gap of 60 mm between them, and there is a rounded-edge blade (approximately 3 mm diameter) that is pushed downwards to press the sample in the middle to bend it. All the equipment touching the sample have rounded edges, so they bend the sample but not cut it or scratch its surface.

The two points at the bottom (in FIG. 5, each point in contact with the sample represent a line/stick that has a touch length 40 mm with the tested product, while the product is 40 mm wide and 90 mm long). The tested products are steady, no movement. The top point in the centre and above the product can move downward (with a “pre-test speed” of 5 mm/s), and compress the centre of the product to bend it (with a “test speed” of 5 mm/s, target distance of 15 mm). Then the top centre point move upward, wait for 5 sec, and move downward again to do the second time compression. Each upward moving was with “post-test speed” of 5 mm/s. Trigger force setting was 5 g. Typical texture profile analysis (also known as “TPA”) was conducted on the compression tests, which projected values such as hardness, springiness, cohesiveness and resilience. The equations and methods about how the texture analyser's software program calculate these values, can be found in the manual of the software, and are known for people skilled in the art.

### Texture Analysis Method

### TPA Using Plate-Plate Compression

Some of the samples (Sample #18C, #21C, #22C, #26C, Reference #Beef (Commercial cooked beef patty), Reference #Plant-methylcellulose (Commercial plant based patty using methylcellulose as binder), and Reference #Tofu (Commercial soy tofu) were analysed with Texture analysis by TPA using plate-plate compression from top. The samples were cut to a flat cuboid (20 mm width, 20 mm length and 10 mm thickness). Detection probe was model “P/36R”, 36 mm Radius Edge Cylinder probe. The illustration of the compressing system including the detection probe, base platform and sample, is illustrated in FIG. 7.

The downward speed before the probe touching the fibre was 5 mm/s (pre-test speed). The speed of compression when the blade touched the fibre was 5 mm/second (test speed) and compression went to a compression depth until 65% of the height of the sample was reached. Then the probe withdraw (move upward) with speed (post-test speed) 5 mm/second. There was a “trigger force” setting, which was set as 5 g in this study. The waiting time between the first and the second compression was 3 s. The Hardness, cohesiveness, springiness, adhesiveness, chewiness, gumminess and resilience values are calculated by the software of the measurement equipment.

### II-3. Stability of Shape of Raw Shaped (but not Cooked) Plant Protein Based Patties Analysed by Bending Tests: Comparing Different Mixing Method and Mixing Time

Sample Preparation: Step 1 to Step 3 were carried out in the same way as Step 1, Step 2 and Step 3 for Sample #1. Step 4 was different from Step 4 of Sample #1.

Step 4. Mixing, a few different options were compared, namely, for sample #31, (A) mixing with hand and spatula to achieve an even distribution of the components in the mass; otherwise samples #32, #33, #34, mixing options (B) 0.5 min machine mixing (for sample #32), (C) 1.5 min machine mixing (for sample #33) and (D) 3 min machine mixing (for Sample #34), respectively, were tested. Mix the mixture of the textured plant protein and dispersed coagulated emulsion with a planetary mixer (such as Hobart brand N50 5-Quart model planetary mixer) equipped with “B” Flat Beater that provides mixing, kneading and shearing power on the mixture, using medium level of speed (124 RPM, round per minute), respectively. As a result, the kneaded mixture (“kneaded mixture” hereafter) is made from this step.

Step 5 and Step 6. in the same way as Step 5 and Step 6 in Experiment II-2.

The texture analysis method chosen was the three position compression bending test which is the same as in Experiment II-2.

A more detailed description of the quality assessment of the uncooked samples and quantitative results are in section “Evaluation of the results of Experiment II”, Table 9.

### II-4. Texture, Elasticity and Shape-Stability of Cooked Plant Protein Based Patties Analysed by Bending Tests

Sample preparation: samples were prepared from Step 7 in experiment II-2.

The texture analysis method chosen was the three position compression bending test which is the same as in Experiment II-2.

### Evaluation of the results of Experiment II (II-1, II-2, II-3 and II-4)

One should only compare data within the same table. The data between different tables should not be compared as testing conditions differ. The results of the comparison of different textured plant proteins in the production of a formed meat-replacement product are shown in Table 5. A cooked patty made of minced beef (20% fat) was used as reference sample (Reference #minced beef). Quality of the cooked samples was assessed TPA assay performed at room temperature.

TABLE 5
Texture analysis, compressing the cooked patty in
the cup by a texture analyser, for samples prepared in
Experiment II-1. Making of Plant Protein Based Patties:
Comparing Different Particles and Textured Proteins

| | | Hardness | Cohesive- | Chewi- | Resil- |
|---|---|---|---|---|---|
| | Material mixed with the binder | (kg) | ness | ness | ience |
| Sample #13 | Textured Vegetable Protein, “Granule” | 21.5 | 42% | 6476 | 11% |
| | (made of legume and cereal, puffy and | | | | |
| | sponge-like) | | | | |
| Sample #14 | Extruded Protein Product “dense flake” | 18.4 | 31% | 5524 | 7% |
| | (made of legume and cereal, dense) | | | | |
| Sample #15 | Textured Protein (Commercial Soy product, | 41.5 | 57% | 24379 | 20% |
| | “Soy Puffy”) | | | | |
| Sample #16 | Grain particles, steel cut oat, “Steel Cut Oat” | 24.8 | 36% | 8791 | 8% |

When cooked patty Reference #minced beef sample made of minced beef was evaluated in the same way, the resulting hardness was 14.0 kg, cohesiveness 44%, chewiness 5220, resilience 14%.

Sample #13 (Granule) and #15 (Soy Puffy) produced cooked patties that have sufficient quality (texture and mouth-feel), and had textural properties that are high in hardness, chewiness, cohesiveness and resilience.

In contrast, as shown in Table 5, Sample #14 (Dense Flake) and #16 (Steel cut oat) were specifically low in cohesiveness and resilience, which showed that these two samples had bad elasticity and integrity. Furthermore, these results were in agreement with our observations that the patties made with Granule and soy puffy (Sample #13 and #15) were sufficiently cohesive and elastic against hand pressing or bending, while the patties made with Dense Flake and Steel Cut Oats (Samples #14 and #16) were soft, doughy and easy to separate apart against hand pressing or bending. And it was difficult to prepare shaped patty sample of Sample #16 (Steel cut oat), because the mixture was very wet, liquid-like, soft and easy to separate apart. The differences between these samples could be attributed to the water absorption property differences. To name an example, Sample #14 (Dense Flake) and #16 (Steel Cut Oat) both absorb water much slower than Sample #13 (Granule) and #15 (Soy puffy). For example, Sample #13 and #15 had higher 10-min water hydration capacity than Samples #14 and #16.

Especially, Samples #14 and #16 have a much lower capability of absorbing water from the disturbed-coagulum. As a result, it was more difficult to make the particles in the disturbed-coagulum firmly attach, stick or bind to the surface of the Dense Flake and Steel Cut Oat. In addition, the disturbed-coagulum remains as a substantially free-flowing suspension and does not turn to paste-like slurry or bin der.

TABLE 6
Texture analysis, three position bending test, results for bending compressing the shaped but uncooked patties in a bending by a texture
analyser, for samples prepared in Experiment II-2. Stability of Shape of Raw Formed (not cooked) Plant Protein Based Patties

| | Hardness | | | | | |
|---|---|---|---|---|---|---|
| Sample#, and main differences in the binder ingredients | (g) | Springiness | Cohesiveness | Chewiness | Resilience | Remark |
| #29, 10% pea protein, 5% oil, 0% Vinegar | <50 | <1% | <1% | <5 | <1% | Poor stability, |
| | | | | | | easy to break |
| #17, 10% pea protein, 5% oil, 1.3% Vinegar, Disrupted- | 231 | 54% | 8% | 10 | 2% | Poor stability, |
| coagulation, disturb during coagulation | | | | | | easy to break |
| #18, 10% pea protein, 5% oil, 3.3% Vinegar, Disrupted- | 229 | 60% | 39% | 53 | 15% | Acceptable |
| coagulation, disturb during coagulation | | | | | | stability |
| #19, 10% pea protein, 5% oil, 3.3% Vinegar, Disrupted | 199 | 55% | 34% | 38 | 12% | Acceptable |
| coagulation, disturb 1 h after acid addition | | | | | | stability |
| #20, 10% pea protein, 5% oil, 3.3% Vinegar, Disrupted | 175 | 56% | 33% | 32 | 10% | Acceptable |
| coagulation, disturb 4 h after acid addition | | | | | | stability |
| #21, 10% pea protein, 5% oil, 3.3% vinegar, Non- | 84 | 53% | 22% | 10 | 3% | Poor stability, |
| Disrupted-coagulation | | | | | | easy to break |
| #22, 10% pea protein, 5% oil, 13.2% Vinegar, Disrupted- | 228 | 50% | 37% | 42 | 12% | Acceptable |
| coagulation, disturb during coagulation | | | | | | stability |
| #23, 10% pea protein, 5% oil, 3.3% Vinegar, Over | 186 | 38% | 26% | 19 | 7% | Poor stability, |
| Disrupted by Homogenization | | | | | | easy to break |
| #24, 12.5% pea protein, 0% oil, 3.3% Vinegar, Disrupted- | 229 | 53% | 16% | 20 | 6% | Poor stability |
| coagulation, disturb during coagulation | | | | | | |
| #25, 12.5% pea protein, 2% oil, 3.3% Vinegar, Disrupted- | 370 | 57% | 17% | 37 | 8% | Acceptable |
| coagulation, disturb during coagulation | | | | | | stability |
| #26, 12.5% pea protein, 5% oil, 3.3% Vinegar, Disrupted- | 303 | 58% | 27% | 50 | 12% | Acceptable |
| coagulation, disturb during coagulation | | | | | | stability |
| #27, 12.5% pea protein, 5% oil, 3.3% Vinegar, emulsion | 284 | 57% | 37% | 61 | 13% | Acceptable |
| uncooked | | | | | | stability |
| #28, Blended Commercial soy tofu | 320 | 0% | 0% | 0 | 0% | Poor stability, |
| | | | | | | easy to break |

The results in Table 6 show that, in order to achieve products having a sufficiently good stability of shape against bending or pressing, for mimicking the phenomenon that the shaped intermediate products may be pressed, bended or vibrated during the movements in the industrial production before they are processed (such as cooked), it is important and necessary to have treatment parameters as presented in the general description of the invention, such as using a disturbed-coagulum as the binder ingredient.

It is in agreement with the result that Sample #18, #19, #20 and #26 had sufficiently good stability of shape against bending or pressing. For these samples, disturbed-coagulum was used as the binder ingredient, and the pH of the controlled-acidified emulsion was within a range between 5.0 and 5.9.

For example, Samples #18, #19, #20 and #26 had each a relatively high resilience value (at least 10%, preferably at least 12%), which represents how well a product recovers its shape after being bent (in other words, “how well a product fights to regain its original height”, which is “calculated by dividing the upstroke energy of the first compression by the downstroke energy of the first compression” according to the TPA method theory introduction, available under citation [4]).

These samples also had sufficient cohesiveness values (no less than 27%, preferably no less than 30%). A higher value of cohesiveness represents a better recovery power of the product after being bent to certain angle. According to the TPA method theory introduction, the cohesiveness value describes “how well a product withstands a second deformation relative to its resistance under the first deformation”, which was calculated by dividing the area of work during the second compression by that during the first compression. Samples #18, #19, #20 and #26 further had sufficient chewiness values, which were above 30%, preferably no less than 38.

Chewiness in this bending test gave an overall indication about how much force and work would be needed to bend or press the sample (shaped and uncooked product) to certain deformation level (15 mm distance), and was calculated by multiplying the hardness value, cohesiveness value and springiness value. Therefore, it was indicated that, sample #18, #19, #20 and #26 can retain their shape substantially unchanged under relatively higher deformation forces like pressing, dropping, vibrating and bending.

Samples #18, #19, #20 and #26 further had sufficient springiness values, which were at least 55%. Springiness value in this bending test indicated how well the product (shaped-and-uncooked patties) can physically springs back after being first time pressed or bent. Springiness was used to indicate “how well a product physically springs back after it has been deformed during the first compression and has been allowed to wait for the target wait time between strokes”, and was calculated by, e.g. dividing the distance of the detected height during the second compression by that during the first compression.

On one hand, Samples #18, #19 and #26 further had a slightly better stability of shape against bending or pressing than Sample #20. On the other hand, when samples were prepared with non-disrupted-coagulation such as Sample #21, they were not as stable as Sample #18, #19, #20 and #26, in terms of much lowered resilience value, much lowered cohesiveness value, much lowered chewiness value and observation during the tests.

When samples were prepared as over-disruption by homogenization (such as Sample #23), they were not as stable as Samples #18, #19, #20 and #26, in terms of much lowered resilience value, lowered cohesiveness value and observation during the tests. When samples were prepared as lack of sufficient acid addition to the emulsion (pH of the emulsion remained higher than 6, such as Sample #29 and #17), they all had further a significantly worse stability of shape against bending or pressing, in terms of much lowered springiness, cohesiveness, chewiness and resilience values.

When the blended commercial soy tofu was used as a binder, such as in Sample #28, the products were the most brittle and unstable against bending or pressing. Sample #28 had very much lower springiness, chewiness, cohesiveness and resilience values than almost all of the other samples, although it had hardness value at a relative high level. This is in agreement with observation result during the analysis that the samples were broken into two parts without any recovery of shape (no “bouncing back” phenomenon) after the first compression (bending), which therefore do not have any resistance force from the sample against the second round compression (bending). This indicates that the sample (patty using blended commercial soy tofu as binder) was easy to break (separate apart) after forming and before cooking. This is a problem for industrial processing, which result in many broken products during normal processes. Or this will set very restrictive requirements for the production process or operation, which need to avoid bending, pressing or dropping of the mass after forming and before cooking.

Eventually, the cooking treatment improves the hardness, cohesiveness and resilience of the mass, so an inappropriate binder may normally be overlooked or not noticed or not understood as the root cause for a high amount of broken products during and after the production process.

In addition to this, there are also other drawbacks of the end product (cooked patty) produced using blended commercial soy tofu as binder. The products are rubbery and/or clearly reminding people of eating tofu or tofu-containing foods, which may not be favourable or acceptable as a formed meat-replacement product that should further be meat-like.

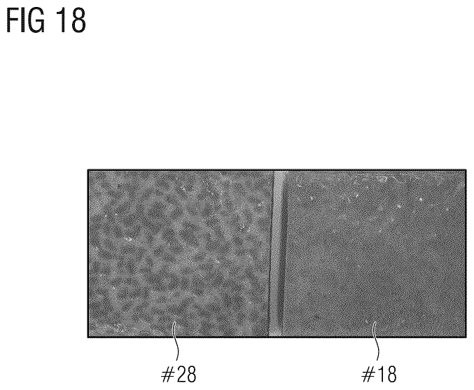

Additionally, such as shown in FIG. 18, the end-product (formed meat-replacement food product) produced using blended commercial soy tofu as binder has more clear tofu gel phase visible in the end product, the gel phase is more bright or lighter colour than the textured protein or other components, even though the tofu was blended to fine particles (less than 1 mm, or less than 0.5 mm) before being used as binder.

The higher value of positive area and hardness represent a higher demand of force needed to bend the cooked product to certain angle. The positive area is the area where the compression force is positive value (showing there is resistance against pressing), the area is a result of the force value multiplied by compression time. Bending of cooked product to certain angle is mimicking the hand touch feel as well as somewhat mouth feel that are related to elasticity and consistency. The proteinaceous emulsion containing oil (such as Sample #25 and Sample #26) were clearly better than proteinaceous solution that did not contain oil before the acidification step. For example, the oil addition increased the resilience value, chewiness, and springiness values.

We found that a sufficient quality of shaped and uncooked patty, namely, resistant and elastic enough against pressing and bending, is a necessary-but-insufficient condition for the successful industrial production of plant-based formed meat-replacement food product. Such quality is favourable and pivotal for product quality, production quality and capacity in industrial food processing, and less restrictive for the production process and operation.

The shaped and uncooked patty should desirably have good stability and intactness of shape after shaping and before cooking, so that it could more easily maintain its shape and quality in a normal and common industrial manufacturing (processing) environment that almost unavoidably has possible shaking, moving, pressing and bending forces being applied on the shaped products, such as at the phases between different processing steps, and at the transferring spaces between different machinery units (such as different processors and different conveying systems). Therefore, processability of Samples #29, #17, #21, #23, #24 and #28 did NOT meet the necessary quality requirements and, therefore, these samples can be determined as insufficient for the successful industrial production of plant-based formed meat-replacement food products.

The further evaluation about cooked patties made under conditions used in the manufacturing of these samples, could be skipped in industrial development consideration, because of the lack of industrial production feasibility.

Nevertheless, in a Research & Development laboratory pilot, it could be possible to apply special care of sample handling and protection (such as ultra-slow and ultra-gentle lifting and movements) on some of the samples processed with such conditions (used in Sample #29, #17, #21, #23, #24 and #28) and therefore allow to conduct certain physical and sensorial evaluation tests on their cooked patty samples. For example, we still worked in laboratory to evaluate Samples #29C, #21C and #23C.

FIG. 18 shows on the left a cooked patty produced using blended commercial soy tofu as binder (Sample #28) and on the right, cooked patty (Sample #18). Sample #28 has more clear tofu gel phase visible in the end-product, the gel phase is more bright or lighter colour than the textured protein or other components.

Sample #18 was manufactured using a proteinaceous binder ingredient made of 12.5% pea protein, 5% oil, 3.3% vinegar, disrupted-coagulation—this sample is more homogenous, the texture protein and binder gel phase were not too much separated, although the mixing, forming and cooking steps were similar for both samples.

TABLE 7
Three-position compression bending test results for different samples. Samples
are shaped and cooked after shaping. Samples prepared in Experiment II-3.

| | Positive area | Hardness | | Cohe- | | Resil- | |
|---|---|---|---|---|---|---|---|
| Sample number and main differences | (kg · sec) | (kg) | Springiness | siveness | Chewiness | ience | Remark on textural analysis |
| #1C, 12.5% pea protein, 5% oil, 3.3% Vinegar, | 2.5 | 1.1 | 80% | 13% | 111 | 8% | Acceptable texture, good |
| Disrupted-coagulation, disturbed during | | | | | | | elasticity, acceptable mouth- |
| acidification | | | | | | | feel quality |
| #18C, 10% pea protein, 5% oil, 3.3% Vinegar, | 2.4 | 1.0 | 74% | 17% | 125 | 9% | Acceptable texture, good |
| Disrupted-coagulation, disturbed during | | | | | | | elasticity, acceptable mouth- |
| acidification | | | | | | | feel quality |
| #20C, 10% pea protein, 5% oil, 3.3% Vinegar, | 1.1 | 0.5 | 67% | 14% | 53 | 7% | Acceptable texture, |
| Disrupted coagulation, disturbed 4 h after acid | | | | | | | acceptable elasticity |
| addition | | | | | | | |
| #21C, 10% pea protein, 5% oil, 3.3% vinegar, | 1.7 | 0.8 | 73% | 13% | 73 | 6% | Poor texture, unacceptable |
| Non-disrupted coagulation | | | | | | | mouthfeel, soft and dough- |
| | | | | | | | like |
| #23C, 10% pea protein, 5% oil, 3.3% Vinegar, | 1.9 | 1.2 | 51% | 5% | 29 | 3% | Poor texture, poor elasticity |
| Over Disrupted by Homogenization | | | | | | | |
| #27C, 12.5% pea protein, 5% oil, 3.3% | 1.4 | 0.9 | 49% | 4% | 15 | 1% | Poor texture, poor elasticity |
| Vinegar, emulsion uncooked | | | | | | | |
| #29C, 10% pea protein, 5% oil, 0% Vinegar | <0.5 | <0.1 | <10% | <5% | <10 | <3% | Poor texture, poor elasticity, |
| | | | | | | | easy to break, too weak to |
| | | | | | | | be held on the analyser |
| # 1T, 12.5% pea protein, 5% oil, 3.3% Vinegar, | 7.9 | 1.6 | 94% | 84% | 1241 | 55% | Acceptable texture, good |
| Disrupted-coagulation, disturbed during | | | | | | | elasticity, acceptable mouth- |
| coagulation, 1% transglutaminase | | | | | | | feel quality |
| #18T, 10% pea protein, 5% oil, 3.3% Vinegar, | 7.9 | 1.7 | 91% | 63% | 973 | 41% | Acceptable texture, good |
| Disrupted-coagulation, disturb during | | | | | | | elasticity, acceptable mouth- |
| coagulation, 0.5% transglutaminase | | | | | | | feel quality |
| #21T, 10% pea protein, 5% oil, 3.3% vinegar, | 3.4 | 1.0 | 87% | 40% | 350 | 22% | |
| NON-disrupted coagulation, 0.5% | | | | | | | |
| transglutaminase | | | | | | | |
| #22T, 10% pea protein, 5% oil, 13.2% Vinegar, | 5.7 | 2.2 | 83% | 20% | 364 | 14% | Bad mouth-feel quality, hard |
| Disrupted-coagulation, 0.5% transglutaminase | | | | | | | and doughy |
| #29T, 10% pea protein, 5% oil, 0% Vinegar, | <0.5 | <0.1 | <10% | <5% | <10 | <3% | Poor texture, poor elasticity, |
| 0.5% transglutaminase | | | | | | | easy to break, too weak to |
| | | | | | | | be held on the analyser |
| #30T, 10% pea protein, 5% oil, 3.3% Vinegar, | 4.4 | 1.9 | 79% | 15% | 229 | 8% | |
| emulsion uncooked, 0.5% transglutaminase | | | | | | | |
| Reference #Beef | 2.4 | 0.7 | 92% | 61% | 365 | 35% | Acceptable texture, good |
| | | | | | | | elasticity |

The results in Table 7 show that, in order to achieve formed meat-replacement food products having a sufficiently good mouthfeel (elasticity) and good stability of shape against bending, it is important to have disrupted-coagulation treatment. It was in agreement with the result that Sample #1C, #18C and #20C had sufficiently good mouthfeel (elasticity) and good stability of shape intactness against bending. These samples were NOT hard or stiff (having low hardness values, between 0.5 and 1.5 kg) against bending compression. And it was not necessary, or it was not even desirable, to require a sample to be stiff or hard against the bending, especially during the first bending compression circle.

The analyser software calculated the Hardness value from the peak force that occurred during the first compression. So a analysed sample having a relatively high hardness value in this test, together with a low springiness, and/or a low cohesiveness, and/or a low resilience values, would often indicated the sample being brittle, stiff and even fragile. More importantly, Samples #1C, #18C and #20C had a relatively high resilience value (larger than 7%), which represents how well a product recovers its shape after being bent. These samples also had sufficient cohesiveness values (at least 13%). The higher value of cohesiveness represents better recovery power of the cooked product after being bended to certain angle.

Bending of cooked product to a certain angle is mimicking the hand touch feel as well as somewhat mouth-feel that are related to elasticity and integrity. Samples #1C, #18C and #20C further had sufficient springiness values, which were above 65%, preferably above 70%.

Samples #1C and #18C further had slightly better texture, mouthfeel and elasticity quality against bending than Sample #20C did.

Furthermore, when samples were prepared with non-disrupted-coagulation such as Sample #21C, they were not as elastic as Samples #1C, #18C and #20C, in terms of lowered resilience value and observation during the tests. When the samples were prepared as over-disruption by homogenization (such as Sample #23C), and/or lack of heating denaturation treatment on the emulsion (such as Sample #27C), and/or lack of acid addition to the emulsion (pH of the emulsion remained higher than 6, such as Sample #29C), they all further had a significantly worse texture, mouthfeel and elasticity quality against bending, in terms of much lowered springiness, cohesiveness and resilience values.

The addition of transglutaminase clearly and largely increased the firmness and elasticity of the cooked patty (higher hardness, higher springiness, higher cohesiveness, higher chewiness and higher resilience). There is a clear synergistic effect between the acid dosage (pH value to be controlled between 5.0 and 5.9), disrupted-coagulation and transglutaminase enzyme addition (enzymatic crosslinking).

For example, when comparing the chewiness values, Sample #18T had very high chewiness value, which was approximately more than 100 times as high as that of Sample #29T, though Sample #29T had only one aspect different from Sample #18T: Sample #29T did not have any acid addition, while Sample #18T was prepared with emulsion containing 10% pea protein and 5% oil, being acidified with 3.3% vinegar, treated with Disrupted-coagulation, disturbed during coagulation, and had 0.5% transglutaminase addition. The chewiness value of Sample #18T was approximately 3 times as high as that of Sample #21T.

Sample #21T had only one aspect different from Sample #18T, which was about that Sample #21T was not treated with disrupted-coagulation.

Sample #18C had the highest chewiness value among the samples that were prepared without adding transglutaminase (Samples #1C, #18C, #20C, #21C, #23C, #27C and #29C).

Nevertheless, the chewiness value of Sample #18T was approximately 8 times as high as that of Sample #18C.

Sample #New18C had only one aspect different from Sample #18T, which was about that Sample #18C did not have transglutaminase addition. Therefore, it can be concluded that using transglutaminase alone for the binding this type of products was insufficient and/or much less effective than using transglutaminase together with treatments of acidification to pH value between 5.0 and 5.9, and disrupted-coagulation.

Samples #22T and #30T were very much worse than Samples #18T and #1T, especially clearly as they had much lower cohesiveness and resilience values. Although the cohesiveness and resilience values might be seen as being higher than those of Samples #1C and #18C, the cohesiveness and resilience values alone did not enable acceptable mouthfeel texture for Samples #22T and #30T, which can be attributed to their high hardness (much higher than that of Samples #1C and #18C). The hard patties need much higher resilience, springiness, cohesiveness, in order to be regarded as palatably chewy and elastic products.

We observed that when hardness value was above 1.5 kg, and/or when chewiness value was higher than 300, the cohesiveness should be above 41% to keep the product's mouth-feel palatably chewy and elastic. If in such condition, cohesiveness value was below 40%, the products can turn to have an undesirable mouthfeel such as dry, stiff, hard, rubbery or dough-like (doughy after being chewed). In addition, the preparation of Sample #30T was much more difficult than preparing samples like Sample #18T, because the shaping and maintaining the shape of the raw (uncooked) patty of Sample #30T was difficult.

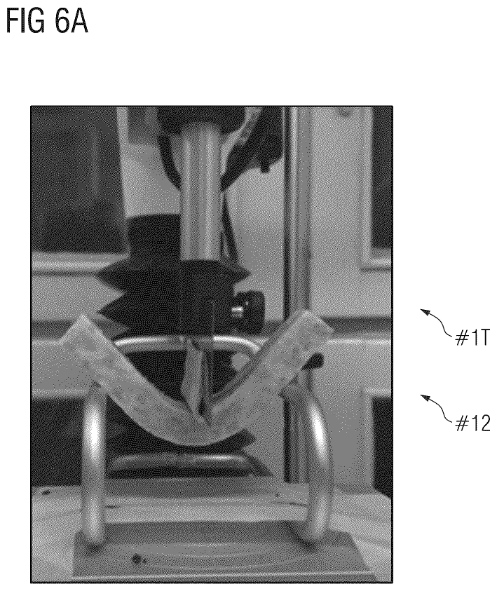

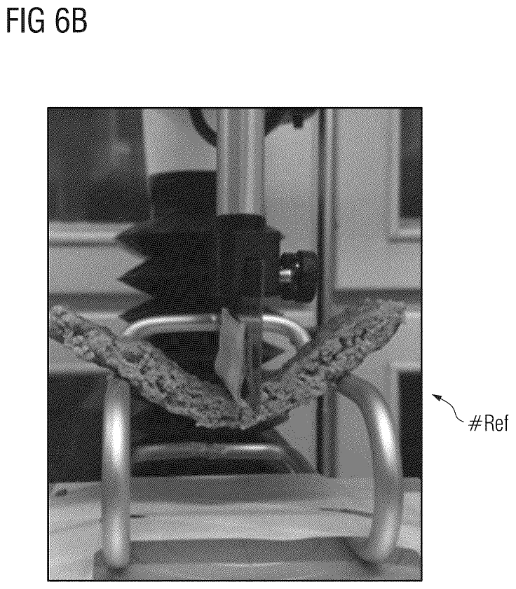

FIG. 6A is a photograph of a cooked patty of Sample #1T during the three-position compression bending test. Sample #1T is similar to sample #12. FIG. 6A shows that the cooked patty of Sample #1T and Sample #12 are elastic, not breaking apart by bending.

FIG. 6B is a cooked beef meat patty (reference #beef) that we used as our reference sample, during the three-position compression bending test.

FIGS. 6A and 6B show that the Sample #1T/Sample #12 and cooked beef meat patty reference #beef had good elasticity and firmness that can hold their shapes and integrity unbroken. The patties could be flexibly bent during the bending test. Cooked beef meat patty, when analysed in the same bending test texture analysis, showed a high springiness value, a high cohesiveness value and a high resilience value, as well as a moderate hardness and chewiness values. That was in agreement with our observation and sensorial preference about texture of the cooked beef meat patty, which should be elastic, cohesive, stable and flexible against pressing, but not too stiff or too hard.

TABLE 8
Texture analysis by plate-plate compression from
top for cooked patty Samples cut to a flat cuboid (20 mm
width, 20 mm length and 10 mm thickness) For samples
prepared in Experiment II-3. The non-commercial Samples are
shaped and cooked after shaping.

| Sample#, and main differences in the binder | Hardness | Spring- | Cohesive- | Chewi- | Resil- |
|---|---|---|---|---|---|
| ingredients | (kg) | iness | ness | ness | ience |
| #18C, 10% pea protein, 5% oil, 3.3% vinegar, | 13.1 | 73% | 38% | 3691 | 13% |
| disrupted-coagulation | | | | | |
| #22C, 10% pea protein, 5% oil, 13.2% vinegar, | 13.1 | 53% | 29% | 1985 | 11% |
| disrupted-coagulation | | | | | |
| #26C, 12.5% pea protein, 5% oil, 3.3% vinegar, | 18.0 | 76% | 43% | 5878 | 15% |
| disrupted-coagulation | | | | | |
| Comparison sample as is: | | | | | |
| Reference #Beef | 12.0 | 88% | 63% | 6639 | 26% |
| Reference #Plant-methylcellulose | 8.8 | 63% | 49% | 2682 | 17% |
| Reference #Tofu | 1.9 | 96% | 42% | 740 | 9% |

The results in Table 8 show that when the texture analysis (TPA) was conducted with this set-up (sample size and geometry), the major differences between a commercial cooked beef patty Reference #Beef (bought from a restaurant) and a commercial soy tofu Reference #Tofu (bought from a supermarket, product name MAMA MEM'S LUOMU TOFU 600G, Ingredient list: Organic soybeans, water, organic vinegar, salt, manufacturer Mama Mem's Oy, Finland) were the much lowered cohesiveness value, hardness value, chewiness value and resilience value. On the other hand, the commercial soy tofu had higher springiness value than the commercial cooked beef patty. These properties made tofu insufficient for being regarded as a satisfactory meat-like meat replacement product. Cooked beef meat patty was more elastic, chewy and firm than tofu.

When too much acid was added (13.2% vinegar added, resulting in emulsion pH below 5.0), such as in Sample #22C, the cooked patty product had lowered cohesiveness, springiness, chewiness and resilience values than the Sample having emulsion pH between 5.0 and 5.9, such as Sample #18C. On the other hand, Samples #22C and #18C had a very similar hardness value. These results were in agreement with the sensorial evaluation observation about these samples that Sample #22C had a worse mouth-feel quality, as being more dough-like, more mush-like, but less elastic, less chewy, less juicy, than sample #18C.

The increase of concentration of pea protein isolate in emulsion from 10% (such as in Sample #18C) to 12.5% (such as in Sample #26C) resulted in increase of firmness and elasticity (in terms of higher hardness, cohesiveness, gumminess, chewiness, and resilience values).

TABLE 9
Texture analysis, three position bending test, results for compressing the shaped
but uncooked patties in a bending by a texture analyser. For samples prepared in
Experiment II-3. Samples were patties mixed with different method of mixing time.

| Sample number, | Hardness | | | | | |
|---|---|---|---|---|---|---|
| Main differences | (g) | Adhesiveness | Springiness | Cohesiveness | Chewiness | Resilience |
| #31, hand mixing | 115 | | 0% | 0% | 0 | 0% |
| #32, 0.5 min machine mixing | 136 | −19 | 37% | 21% | 11 | 5% |
| #33, 1.5 min machine mixing | 213 | −9 | 43% | 31% | 28 | 9% |
| #34, 3 min machine mixing | 264 | −7 | 47% | 38% | 47 | 12% |

Table 9 shows that the machine kneading mixing results in a dramatic increase of binding effect (springiness, cohesiveness, chewiness and resilience of the shaped-and-uncooked products). Moreover, the longer the mixing time, the higher strength or stability of the shaped-and-uncooked products was achieved.

Hand mixing of the textured plant-based proteins and disturbed coagulum allowed an even distribution of the components. However, this method was clearly inadequate for generating a cohesive raw mass under normal hand mixing conditions. Despite of continuing hand mixing over an extended time, the cohesiveness of the mix did not improve. Thus, it was clear that the level of energy input intensity of hand mixing was insufficient to make use of the specific bonding structure (potential power) of the disturbed-coagulum to allow the binding of the TVP.

Mixing for 0.5 min resulted in a very weak structure that could barely hold together its poorly shaped structure. Therefore, a patty produced in this manner is easy to break apart and would require extreme care during manufacturing. On the industrial level a raw patty should be able to withstand several stresses such as shaking, moving, pressing, etc. Therefore, this type of raw patty is unsuitable for large scale manufacturing as it would result in a very large percentage of rework/food waste.

To be able to produce a raw patty that can withstand the rigour of industrial scale production, shearing forces should be applied to the mix of textured plant-based proteins and the disturbed-coagulum, so that a proteinaceous bonding network can be developed.

### III Theoretical EXPERIMENTS and CONCLUSIONS

We arranged a few test groups to test the theory, and present our conclusions after studying the combined test results and observations from different aspects:

### III-1 Observation and Analysis of the Microstructure, Type, and Properties of the Proteinaceous Binder System—Suspension Containing Self-Encapsulated Particles

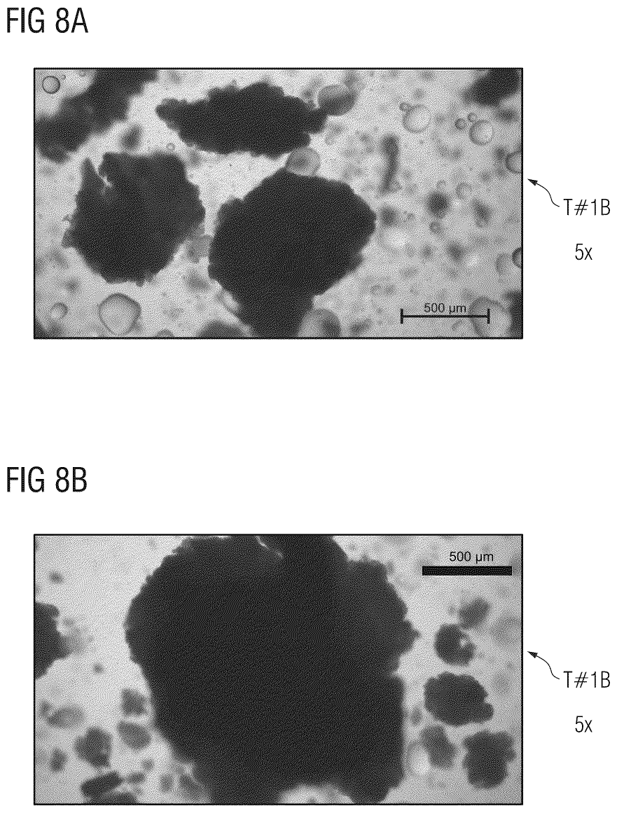

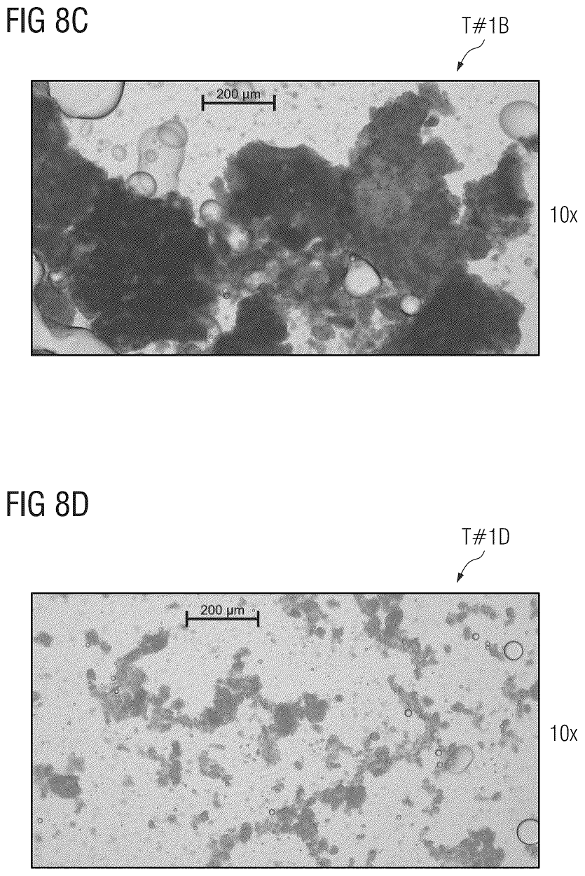

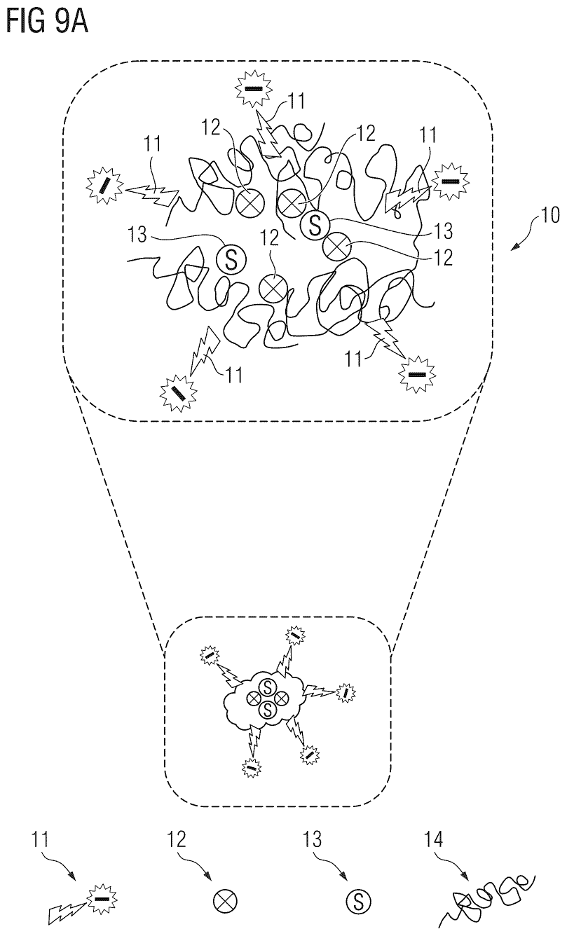

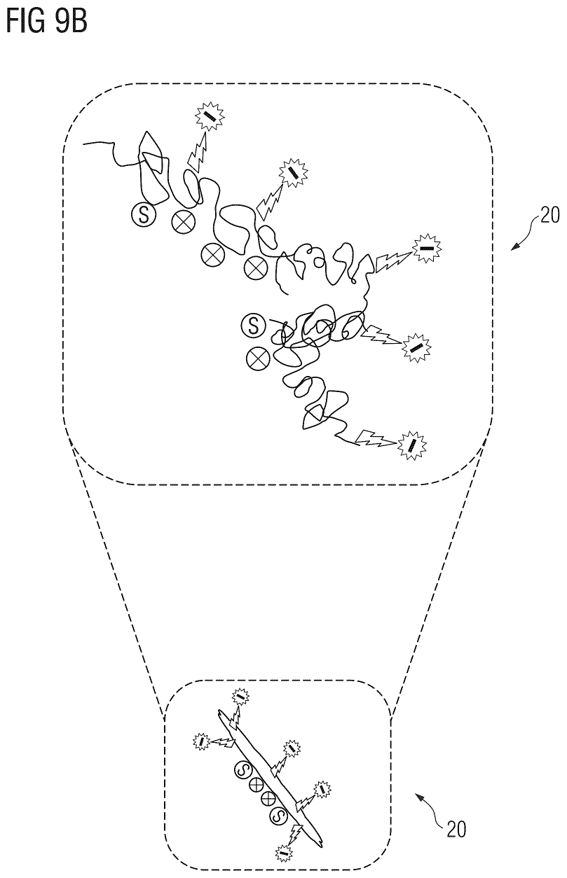

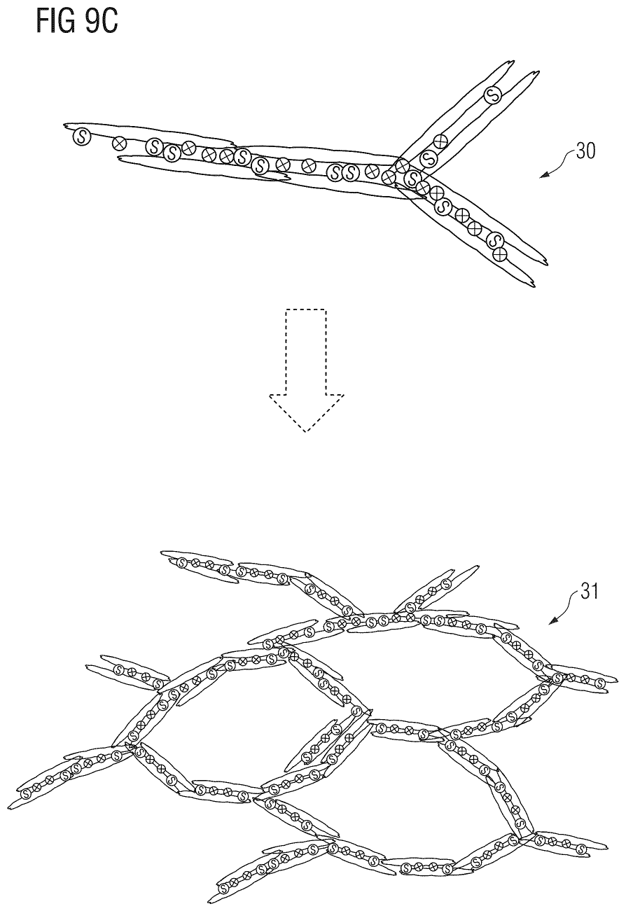

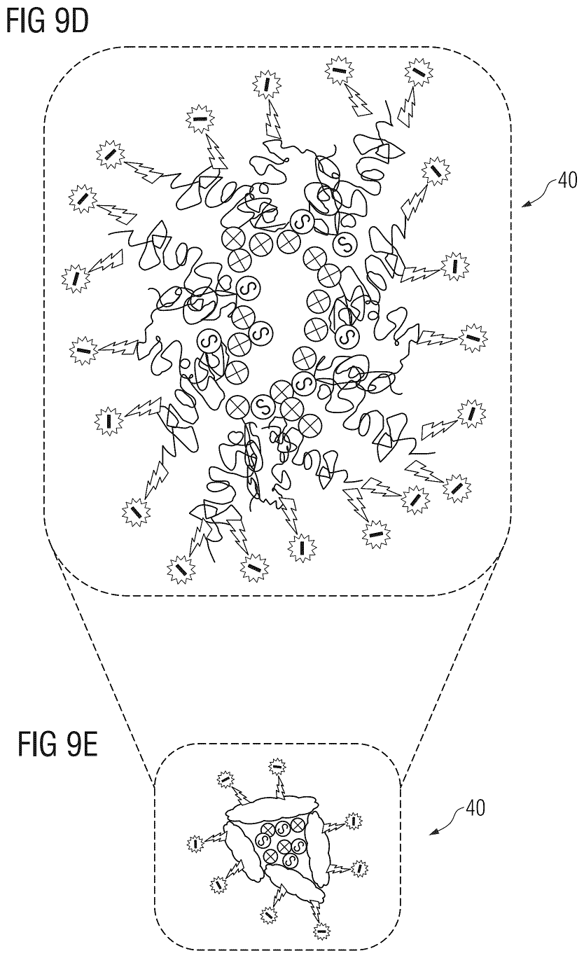

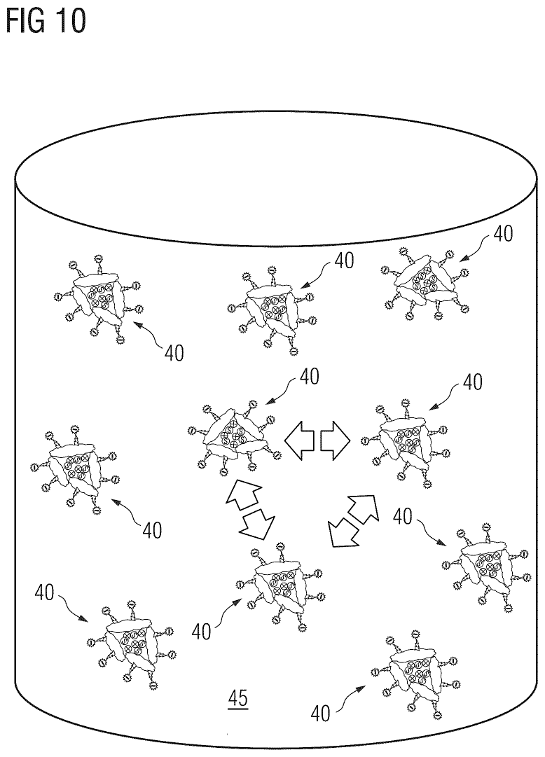

The process (emulsion+denaturisation+acidification to pH 5.0-5.9+disrupted-coagulation) results in an unique suspension, which substantially contains “self-encapsulated binder particles” having a particle size larger than 0.101 mm and smaller than 10 mm, preferably between 0.3 mm and 5 mm, mostly between 0.3 mm and 2 mm. The appearance and microstructure of the suspension, especially of “self-encapsulated binder particles” can be seen in structure 3 in FIG. 2, as well as in FIG. 3A, 3B and in microscopy images FIG. 8A, 8B, 8C and also in the schematic illustration in FIG. 9D, FIG. 10.

The “self-encapsulated binder particle” is different from traditional art and is not known to the inventors from other kinds of treatments of proteinaceous material, such as, (a) traditionally coagulated gel, such as tofu; (b) homogenized gel, such as plant-based yogurt or our reference sample of homogenized gel made from the same composition as the self-encapsulated binder particles, for example. The self-encapsulated binder particles are the best binder ingredient known to the inventors to be used in the manufacture of formed meat-replacement products such as patties, and they perform significantly better than the (a) traditionally coagulated gel, such as tofu; (b) homogenized gel.

Difference Aspect #1, Particle Size:

The self-encapsulated binder particles have a particle size substantially smaller than the particles in a coagulated gel curd like tofu, or the intermediate coagulum cluster (having particle size preferably larger than 10 mm) in tofu making. The particle size is also substantially larger than the homogenized gel, such as plant-based yogurt or our reference sample of homogenized gel, which have a particle size between 0.1 μm and 0.2 mm.

Experiment III-1-A on Theory Test Samples T #1A, T #1B, and T #1D.

Differences in macrostructural and microstructural properties of different types of suspensions.

Sample Preparation:

Step 1. We followed the Step 2.a. of Sample #1, however, varied the mixture recipes: pea protein 10% (as-is weight)—oil 5% (as-is weight), water 85% (as-is weight).

Step 2. We followed the Step 2.b. of Sample #1,

Step 3. We followed the Step 2.c. of Sample #1, however, varied the treatment on coagulation: for some samples (Theory test sample T #1A), the vinegar was mostly evenly distributed in the cooked emulsion by very gentle stirring and shaking, which did not disturb the coagulation process. It did not break most of the formed large protein clusters (size no less than 10 mm, preferably between 10 mm and 90 mm). As a result, the Theory test Sample T #1A was coagulum that was not a disturbed-coagulum. Differently, for some other samples (Theory test sample T #1B) the coagulation was disturbed in the same way as in Step 2.c. of Sample #1, and the disturbing treatment was conducted starting from the time when the acid was being added. For some other samples (Theory test sample T #1D), the coagulation was over-disrupted by homogenizing the mixture using a high speed blender (such as kitchen hand-held immersion blender, equipped with rotating knife blades), after the coagulation was disturbed in the same way as in Step 2.c. of Sample #1.

Step 4. The acidified emulsions were observed by photographing and by observing an optical microscope having different magnifications available.

The macrostructural properties in the results were observed and photographed. The results are presented in FIGS. 2, 3A and 3B.

The microstructural property results observed by an optical microscope are presented in FIG. 8A to 8D.

FIGS. 8A and 8B are microscopic images of Theory test sample T #1B, observed with an optical microscope with a 5 times magnification. A few drops of samples were very gently spread in a few drops of water on top of the observation specimen glass before observation, so the microstructure of the samples was substantially not changed during observation.

FIGS. 8A and 8B show particles in a suspension. The particles have a particle size between 0.2 mm and 0.6 mm in FIG. 8A and between 0.15 mm and 1.8 mm in FIG. 8B. The particles are mostly random shaped, and are more frequently oval shaped, and often are round shaped.

FIG. 8C is a microscopic image of Theory test sample T #1B, observed with an optical microscope with 10 times magnification. A few drops of samples were very gently spread in a few drops of water on top of the observation specimen glass before observation, so the microstructure of the samples was substantially not changed during observation. FIG. 8C shows particles in a suspension. The particles have particle sizes larger than 0.5 mm. The particles comprise or are composed of dense tightly bound solid materials, which are protein molecules.

FIG. 8D is a microscopic image of Theory test sample T #1D, observed with an optical microscope equipped with a lens having 10 times magnification. A few drops of samples were very gently spread in a few drops of water on top of the observation specimen glass before observation, so the microstructure of the samples was substantially not changed during observation. The image shows particles in a suspension. The particles are substantially small, round particles smaller than 0.1 mm in diameter, though they may randomly and loosely attach to each other and may therefore be observed as a narrow chain shape. The narrow chain has a length less than 0.5 mm, and thickness less than 0.1 mm, more frequently less than 0.05 mm.

Without willing to be bound by theory, we try in the following to explain the microstructural properties revealed by the experiments on our samples, on the basis of our observation and theoretical knowledge of the inventors.

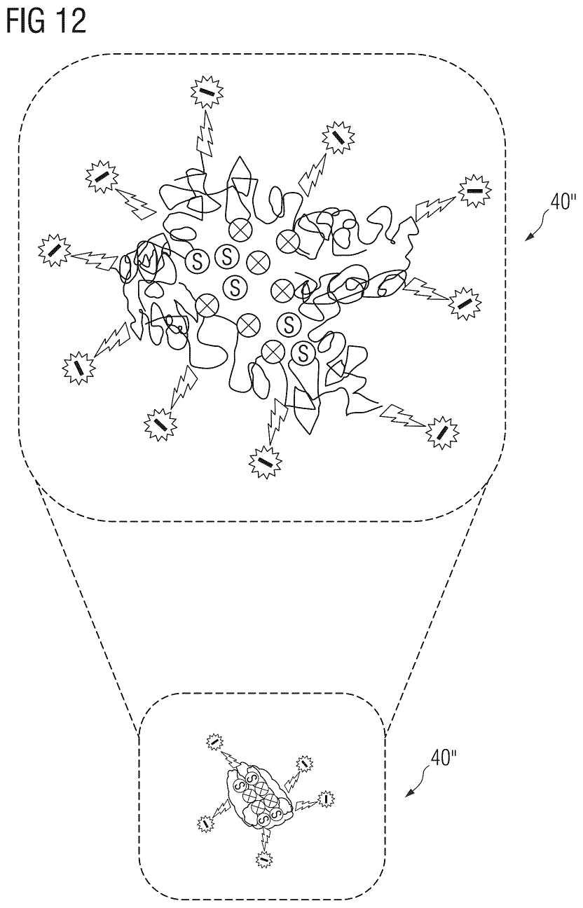

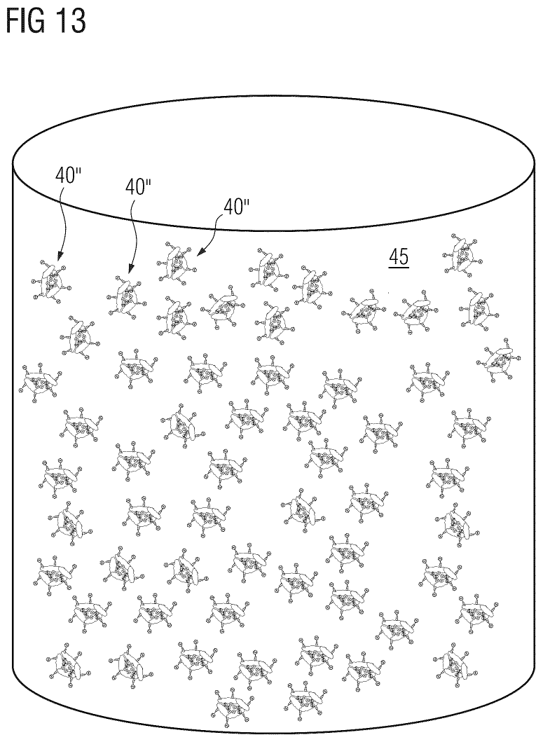

While FIG. 9A-9C describe background art, FIG. 9D, FIGS. 9E, and 10 are believed to present the theoretical model explaining the observations for Theory Test Sample T #1B, while FIG. 12 and FIG. 13 are believed to explain the observations for sample_Theory Test Sample T #1D. The actual number of the positively or negatively charged sites are only for illustrative purposes. The fact that some of the magnifications show a different number of positively or negatively charged sites, for example, is the result of artistic illustration only and should be neglected.

FIG. 9A illustrates a typical plant protein molecule 10, such as pea protein, which is native and not denaturized, in a theoretical model, with its magnification. The protein molecule has peptide chains 14, which make up a particle, frequently a globular-shaped particle. There are electronic net charged amino acid side chains 11 on the surface of the protein molecules, which are negatively charged. There are hydrophobic sites (amino acid side chain groups) 12 and Sulphur-containing amino acid sites (amino acid side chain groups, either free sulphur group, or disulphide-bonds) 13 being embedded in the inner side of the protein molecules.

For the sake of clarity, amino acid side chains 11, hydrophobic sites 12, Sulphur-containing amino acid sites 13, and peptide chains 14 are shown below the sketch separately, to illustrate how they are represented in the theoretical model.

FIG. 9B illustrates a typical plant protein molecule 20, such as pea protein, which is heat denaturized, in a theoretical model, with its magnification. Compared to the native and not denaturized typical plant protein molecule shown in FIG. 9A, the structure of the heat denaturized plant protein molecule 20 is unfolded, or opened, or extended. There are still electronic net charged amino acid side chains 11 on the surface of the protein molecules, which are negatively charged. The hydrophobic sites (amino acid side chain groups) 12 and Sulphur-containing amino acid sites 13 (amino acid side chain groups, either free sulphur group, or disulphide-bonds) being exposed from the inner side of the protein molecules 20 to the surface.

FIG. 9C illustrates above a typical plant protein heat-plus-acid-induced non-disrupted coagulation initiation process, in which the heat denaturized protein molecule 30 is in unfolded shape under an environment that has pH value close to the pI (isoelectric point) of the proteins. This is the situation in which the protein molecules 30 can move close to each and then aggregate, and further form protein-protein interaction bonds, such as hydrophobic interaction, disulphide bonds and hydrogen bonds. There is a clear trend of ordered aggregation and bond formation. The sketch below it, represented a zoom out view of continued coagulation. There is a clear continued trend of ordered aggregation and bond formation. As a result, the crosslinked proteins start to form a highly ordered broad continuous network structure 31. Such network structure 31 normally has a stronger gel strength than gels having randomly formed network structure.

FIG. 9D illustrates a self-encapsulated proteinaceous particle 40, in a theoretical model, with its magnification. Self-encapsulated proteinaceous particle 40 is the result from a typical plant protein heat-plus-acid-induced disrupted-coagulation initiation process, in which the heat denaturized protein molecules in unfolded shape under an environment that has pH value close to the pI (isoelectric point) of the proteins (or has pH value between 5.0 and 5.9; or 0.5-1.4 in the pH scale above the pI of the protein) have moved close to each other, aggregated, and further formed protein-protein interaction bonds, such as hydrophobic interaction, disulphide bonds and hydrogen bonds. The situation in FIG. 9D is different from the situation illustrated in FIG. 9C mainly because the disrupting of the coagulation causes disturbing forces, which in consequence cause the protein molecules aggregation and bond formation become random.

As a result, there is no highly ordered broad continuous network structure formed. Instead, the proteins aggregated protein molecules form particles (we refer to them as self-encapsulated proteinaceous particles 40) that have a special structure in which the hydrophobic sites 12 and Sulphur-containing amino acid sites 13 of the protein crosslink to each other and get embedded in the inner side of the particles 40. On the other hand, the hydrophilic and negatively charged amino acids 11 are located on the surface of the particles 40. Such particles 40 are larger than emulsion drops, and are larger than 0.101 mm and smaller than 10 mm, preferably between 0.3 mm and 5 mm, more preferably between 0.3 mm and 2 mm.

FIG. 10 illustrates a suspension 49 containing self-capsulated proteinaceous particles 40 and water 45. The suspension has pH value between 5.0 and 5.9. In such suspension, the self-capsulated proteinaceous particles 40 have repulsive force between each other, and keep distance to each other, thanks to their similarly charged surface net charge.

Difference Aspect #2, Binding Property:

The self-encapsulated proteinaceous particles 40 do not or do very little binding to each other between different particles 40, when they are simply pressed to touch each other, such as draining or centrifugal dehydrating. The reason for this could be that their active-binding sites (amino acid side chains 11 and unfolded peptide chains 14) are embedded or encapsulated in the centre of the particles 40, such “active-binding sites” are assumed to be more hydrophobic sites, so they tend to aggregate and interact with each other and stay away from water phase. On the other hand, the surface area or surface layer of the particles 40 are apparently hydrophilic and are electronically charged with a negative charge, because the environment (water phase) of the suspension is pH 5.0-5.9 and above isoelectronic point (4.5) of the selected proteins, so it allows the particles to have a certain electronic charge on the surface area. And due the similarly charged situation of each particle 40, the particles 40 have electronically repulsive force between each other, and hence, they can not bind to each other easily. This poor binding property make such self-encapsulated binder particles very unsuitable for making curd products like tofu or cheese, with considering existing knowledge about tofu making.

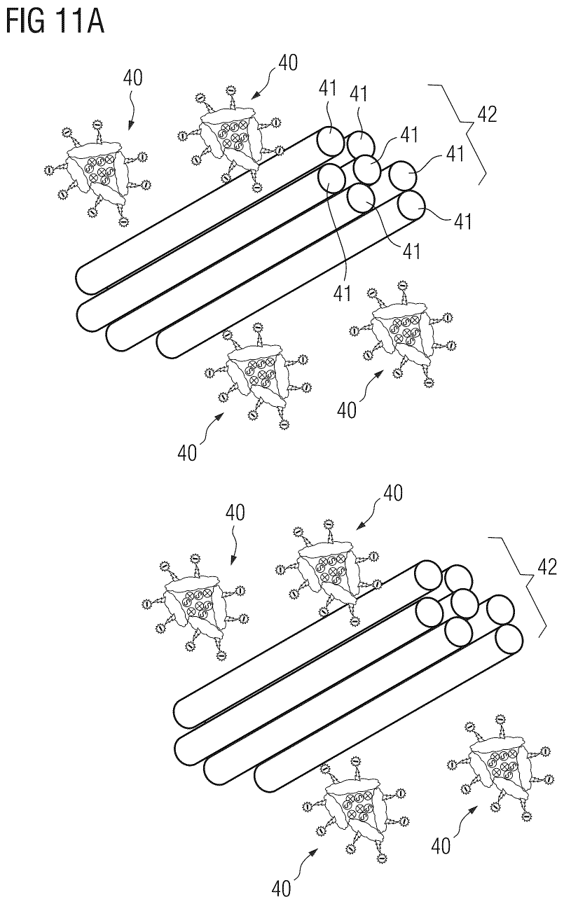

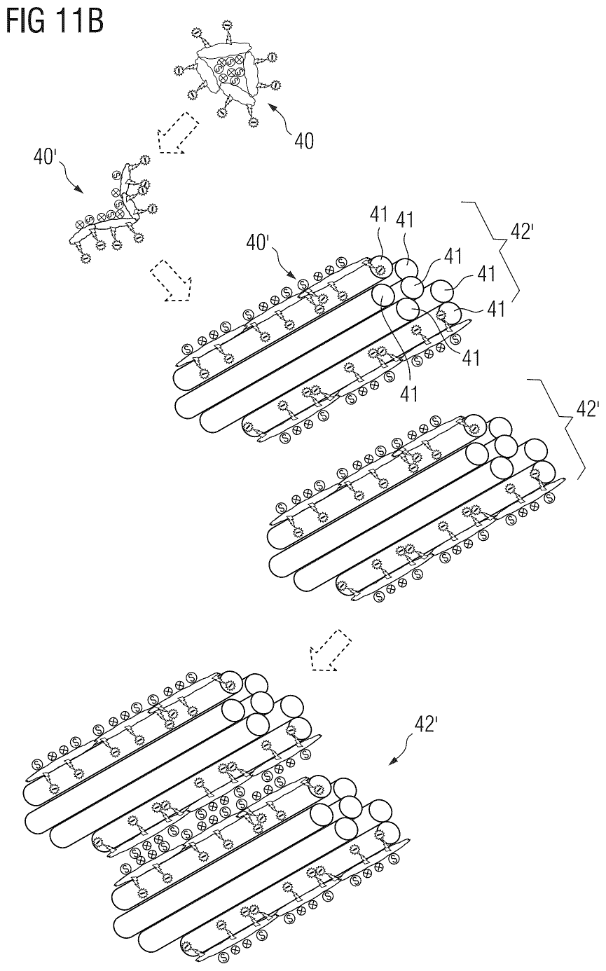

Furthermore, as shown by results in Table 9, self-encapsulated proteinaceous particles 40 have a little binding power when they are simply mixed with the textured plant protein by hand to achieve an even distribution of the mixture (cf. Sample #31): The resulting mixture had weak intactness or strength against bending. We try to explain this observation theoretically with reference to FIG. 11A.

FIG. 11A is a sketch of a theory model with which we try to explain the mechanism leading to the poor binding that was observed in the mixture containing a textured vegetable protein and self-encapsulated proteinaceous particles 40 when they were simply mixed with the textured plant protein by hand to achieve an even distribution of the mixture. The textured vegetable proteins have long continuous proteinaceous fibres 41 that are substantially aligned and crosslinked to each other to form protein bundles 42.

Nevertheless, surprisingly, we found that, the self-encapsulated proteinaceous particles 40 can and will express significantly higher and more sufficient binding power when they are mixed with dry textured protein (dry textured vegetable protein manufactured with low-moisture protein texturization extrusion) and mixed with kneading and shearing power that is sufficient to break or open such self-encapsulated binder particles 40 to expose the “active-binding sites” (or “active-binding and hydrophobic sites”).

FIG. 11B is a sketch of a theory model with which we try to explain the mechanism leading to the good binding that was observed in the mixture containing a textured vegetable protein and self-encapsulated proteinaceous particles 40 when they were simply mixed with the textured plant protein with kneading and shearing power that is sufficient to break or open the self-encapsulated binder particles 40 to expose the “active-binding sites”, and to achieve an even distribution of the mixture.

With the sufficient kneading and shearing power, self-capsulated proteinaceous particles 40 can apparently be unfolded or opened to expose their embedded hydrophobic zones and sulphur-group containing sites from inner side to surface, when they are sheared or kneaded together with materials like textured vegetable protein. The unfolded or opened self-capsulated proteinaceous particles 40′ can bond to the textured vegetable protein bundles 42, especially their hydrophilic sites. Because the textured vegetable protein absorbed the water 45 from the suspension 49 and became wet, they can attract the hydrophilic zones of the unfolded self-capsulated proteinaceous particles 40′. Then the textured vegetable protein 41′ with the unfolded self-encapsulated proteinaceous particles 40′ can crosslinks via the unfolded self-encapsulated proteinaceous particles 40′ to other textured vegetable proteins 41′ also bonded to self-capsulated proteinaceous particles 40′, through hydrophobic interaction, disulphide bonds and other protein-protein interaction. From this, a TVP-Particle-Particle-TVP sandwich structure 42″ is formed (textured vegetable protein—unfolded self-capsulated proteinaceous particles-unfolded self-capsulated proteinaceous particles—textured protein). Furthermore, the whole mixture of TVP and “particles” became crosslinked and having gel strength, biting resistance and bending resistance.

In opened self-encapsulated proteinaceous particles 40′, the surface area (surface layer) can attach to the surface of the textured proteins, and these “active-binding sites” (or “active-binding and hydrophobic sites”) and bind with each other. And in this way, the whole mixture will become interlinked, some crosslinked matrix within the whole mixture is formed. The mechanism of this process was illustrated in FIG. 11A and FIG. 11B. The assumption about the possible mechanism of this process is supported by observation and analysis results such as that being shown in Table #New9 that the machine kneading mixing resulted in dramatic increase of binding effect (springiness, cohesiveness, chewiness and resilience of the shaped-and-uncooked products). Moreover, the longer the mixing time, the more the strength and the stability of the shaped-and-uncooked products increased.

If self-encapsulated binder particles 40 are broken too early, like being broken in the suspension before they are mixed with the textured vegetable protein, such as by homogenization treatment, to get homogenized gel (such as plant-based yogurt or our reference sample of homogenized gel), such as in the case of Sample #8 (“Coagulation and over-disrupted by homogenization”), resulting in structure 4 shown in the photograph of FIG. 2. Water 45 in the suspension can help the homogenizing force to disrupt the self-encapsulated binder particles 40 into much smaller particles 40″ (the smaller particles 40″ may also be referred to as self-encapsulated proteinaceous particle after over-disrupted-coagulation), and maintain their small size, and further make the smaller sized particles 40″ electronically repulsive from each other. Thus, it will not be possible for the smaller particles 40″ to form strong gel with the whole suspension (strong interaction between individual particles). The microscopic observation of the particles is shown in FIG. 8D.

The theoretical model for the over-disrupted-coagulation is presented in FIG. 12 and FIG. 13. In FIG. 12 a particle 40″ produced by homogenizing the particles 40 from FIG. 9D is illustrated in a theoretical model, with its magnification. The homogenization was carried out in the suspension 49, which had water 45 surrounding the proteinaceous particles 40. The water 45 helps to disturb and refold the proteinaceous particles, because of the amphiphilicity of the particles 40 and protein molecules. The particle 40″ in FIG. 12 is smaller than that in FIG. 9D. The particles 40″ have a size close to emulsion drops, though, and substantially have a size between 0.1 μm and 200 μm, more frequently between 1 μm and 100 μm, and most often between 1 μm and 50 μm. When observed under microscope, particles 40″ may randomly and loosely attach to each other to be observed as a narrow chain shape. The narrow chain has a length less than 0.5 mm, and thickness less than 0.1 mm, more frequently less than 0.05 mm. The homogenized particles have the hydrophobic zones and sulphur-containing amino acid zones of the protein crosslink to each other and get embedded in the inner size of the particles. On the other hand, the hydrophilic and negatively charged amino acids are located on the surface of the particles. FIG. 13 illustrates a theoretical sketch of the particles 40″ in suspension.

There is enough scientific knowledge to assume that the broken self-encapsulated binder particles 40″ make more of the comprising proteins self-assembled (folded), which means that those proteins fold on their own to embed their hydrophobic sites in the protein molecular tertiary structure and or secondary structure. This assumption is supported by our observation that the homogenised gel has very good water holding capacity, meaning very good phase stability, meaning very little phase separation between the small (homogenized) particles and the water phase. That shows the small (homogenized) particles 40″ have very good hydrophilicity and net charge (electronic charge) on the particle surfaces; and have their hydrophobic sites embedded inside the molecular structure. And because of this different structure, the small (homogenized) particles 40″ cannot do the so mentioned transformation (unfolding, stick to textured protein, and form interactions between the originally embedded active sites or hydrophobic sites), for example. The shearing or mixing power would hardly be enough to unfold the molecular structure and expose the active site (and/or hydrophobic sites).

Difference aspect #3, overall consistency (gel strength) of the whole suspension, detected by compressing the whole suspension in a cup to a distance of 60% of its original height.

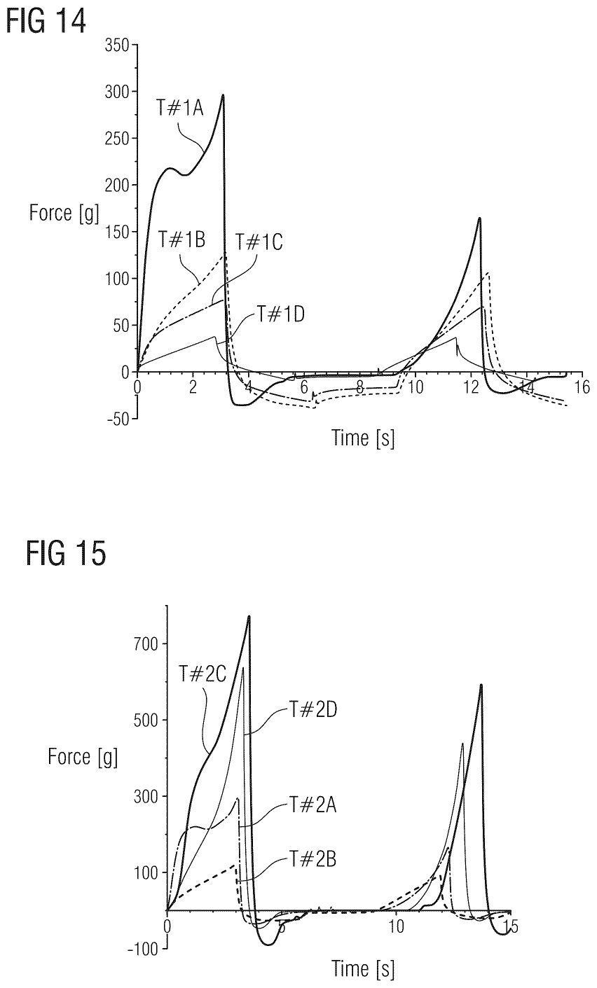

As shown by results in the Table #10 and in FIG. 14, the overall protein curd-like gel (overall structure of the whole mixture) made by non-disrupted-coagulation (Theory Test Sample T #1A) was the most rigid and brittle gel, which has the highest positive area value (around 780 g×sec) and lowest cohesiveness value (around 29%). As can be seen in FIG. 14, the protein curd-like gel made by non-disrupted-coagulation was a gel for which surface breaking occurred when it was penetrated (pressed by a cylinder from the top), as it had a peak force (a separated peak, appeared at time around 1 s) before reaching peak positive force (at time around 3 s). The suspension containing self-encapsulated binder particles made by our invented process (including disrupted-coagulation) (Theory Test Samples T #1B, T #1C) is a weak gel and substantially free-flowing suspension, which had medium level positive area value (around the range between 300 and 420 g×sec) and medium level cohesiveness value (around the range between 68% and 72%). The homogenized gel (such as plant-based yogurt or our reference sample of homogenized gel) (Theory Test Sample T #1D) was the weakest gel and the most free-flowing suspension, which had the smallest positive area value (around 140 g×sec) and medium level cohesiveness value (around the range between 80% and 90%).

As can be seen in FIG. 14, for Theory Test Samples T #1B, T #1C, T #1D the surface breaking did not occur, as their force curve shows a gradual increase until reaching the peak positive force. This surface breaking phenomenon shown in texture analysis force-time curve could be used to identify whether the coagulation was disturbed.

Experiment III-1-B on Theory Test Samples T #1A, T #1B, T #1C, and T #1D. Differences in whole suspension's consistency (gel strength) of different types of suspensions.

Step 1. We followed the Step 2.a. of Sample #1, however, varied the mixture recipes: pea protein 10.0% (as-is weight)—oil 5.0% (as-is weight), water 85% (as-is weight).

Step 2. We followed the Step 2.b. of Sample #1, however, varied as we filled the emulsions from Step 1. in a porcelain baking cup having a cylinder inner shape, diameter of 90 mm, height/depth 55 mm and cooked it autoclave oven at 115° C. for 10 min, in order to reach temperature of the emulsion to be above 98° C. for more than 5 min. The amount of emulsion in each cup was selected to reach the analysis target weight-per-cup, which was described in the detailed description about the texture analysis method and in the explanation about the texture analysis results.

Step 3. We followed the Step 2.c. of Sample #1, however, varied the treatment on coagulation: for some samples (Theory test sample T #1A), the vinegar was mostly evenly distributed in the cooked emulsion by very gentle stirring and shaking, which did not disturb the coagulation process. It did not break most of the formed large protein clusters (size no less than 10 mm, preferably between 10 mm and 90 mm). As a result, the Theory test sample T #1A was coagulum that is non-disturbed; differently, for some other samples (Theory test sample T #1B) the coagulation was disturbed in the same way as in Step 2.c. of Sample #1, and the disturbing treatment was conducted starting from the time when the acid was being added; for some other samples (Theory test sample T #1C), the coagulation was disturbed in the same way as in Step 2.c. of Sample #1, and the disturbing treatment was conducted starting from 1 hour after the time when the acid was being added; for some other samples (Theory test sample T #1D), the coagulation was over-disrupted by homogenizing the mixture using a high speed blender (such as kitchen hand-held immersion blender, equipped with rotating knife blades), after the coagulation was disturbed in the same way as in Step 2.c. of Sample #1.

Step 4. The acidified emulsions were analysed as being contained in the same cup as in Step 2. The acidified emulsions from Step 3. were chilled to below 6° C. for at least 10 min, then tempered to room temperature, and finally evaluated by a texture analyser.

Analysis of the Samples and Results:

We evaluated theory test samples T #1A, T #1B, T #1C and T #1D with a texture analyser equipped with a detection probe (model “P/36R”) that is a 36 mm Radius Edge Cylinder, supplier Stable Micro Systems), which pressed the suspension or curd-like gel (from Step 3.) twice from the top to 60% strain (compression depth, also known as compression distance, was 60% of the original height of the sample). Typical texture profile analysis (also known as “TPA”) was conducted on the compression tests. Before the analysis, the samples were tempered to room temperature around 20° C. The samples were analysed as being contained in the same cup as in Step 2. The illustration of the compressing system including the detection probe, base cup-and-platform and sample, was show in FIG. 4. The amount suspension in a cup was 150 g for each sample. The results are shown in Table 10.

FIG. 14 shows the measurement results for Theory Test Samples T #1A, T #1B, T #1C, and T #1D of Experiment III-1-B. Clearly, there are differences in the suspension consistency (gel strength). To summarize, the samples were:
Theory Test Sample T #1A: non-disrupted coagulation;
Theory Test Sample T #1B: disrupted coagulation, disturbing coagulation during acidification;
Theory Test Sample T #1C: disrupted-coagulation, disturbing coagulation in 1 hour after acidification;
Theory Test Sample T #1D: over-disrupted-coagulation, homogenizing after acidification.

TABLE 10
Texture analysis results of compression tests
conducted on texture of emulsions after acidification.
Comparing whole suspension's consistency (gel strength) of
different types of suspensions.

| | Positive Area | Cohesive- |
|---|---|---|
| Theory Test Sample #, main differences | (g x sec) | ness |
| T#1A, Non-disrupted coagulation | 783 | 29% |
| T#1B, Disrupted-coagulation, disturbing coagulation during | 412 | 68% |
| acidification time | | |
| T#1C, Disrupted-coagulation, disturbing coagulation in 1 hour after | 308 | 72% |
| acidification | | |
| T#1D, Over-disrupted-coagulation, homogenizing after acidification | 136 | 87% |

Results presented in Table 10 were already discussed above, in the first paragraph of section “Difference aspect #3, overall consistency (gel strength) of the whole suspension”. A higher value of positive area represents a higher demand of force needed to bend the suspension or gel to certain percentage of its original height. Positive area is the area where the compression force is positive value (showing there is resistance force against pressing), the area is a result of the force value multiplied by compression time.

Gel strength of suspension made from homogenization is weaker that made from disturbed-coagulum, and further weaker than non-disrupted-coagulation.

Experiment III-1-C on Theory test samples T #2A, T #2B, T #2C, T #2D and T #2E. Effect of disturbing during coagulation and the amount of acid on the gel strength of the coagulum.

Step 1. We followed the Step 2.a. of Sample #1, however, varied the mixture recipes: for some samples, the mixture recipe were (Theory test samples T #2A, T #2B, T #2C and T #2D), pea protein 10.0% (as-is weight)—oil 5.0% (as-is weight), water 85% (as-is weight). But for some other samples (Theory test sample #2E), the mixture recipe pea protein 12.5% (as-is weight)—oil 5.0% (as-is weight), water 82.5% (as-is weight).

Step 2. We followed the Step 2.b. of Sample #1, however, varied it by filling the mixture from Step 1. in a porcelain baking cup having a cylinder inner shape, diameter of 90 mm, height/depth 55 mm and cooked it autoclave oven at 115° C. for 10 min, in order for the emulsion to have a temperature at above 98° C. for more than 5 min. The amount of mixture in each cup was selected to reach the analysis target weight-per-cup, which was described in the detailed description about the texture analysis method and in the explanation about the texture analysis results.

Step 3. We followed the Step 2.c. of Sample #1, however, varied the added vinegar: for some samples (Theory test samples T #2A, T #2B and T #2E), the amount of the added vinegar was 3.3% of the cooked emulsion (as-is weight); or for some other samples (Theory test samples T #2C and T #2D), the amount of the added vinegar was 13.2% of the cooked emulsion (as-is weight). Furthermore, for some samples (Theory test samples T #2A and T #2C), the vinegar was mostly evenly distributed in the cooked emulsion by very gentle stirring and shaking, which did not disturb the coagulation process. It did not break most of the formed large protein clusters (size no less than 10 mm, preferably between 10 mm and 90 mm).

As a result from this step, Theory test samples T #2A and T #2C were coagulum that was non-disturbed. In contrast, for some other samples, Theory test samples T #2B, T #2D and T #2E) the coagulation was disturbed in the same way as in Step 2.c. of Sample #1.
Step 4. For some samples (Theory test samples T #2A, T #2B, T #2C and T #2D), the acidified emulsions from Step 3 were chilled to below 6° C. for at least 10 min, then tempered to room temperature, and finally evaluated by a texture analyzer.
Step 5. For some other samples (Theory test sample T #2E), the acidified emulsions from Step 3. were cooked in autoclave oven at 115° C. for 10 min, in order to reach temperature of the emulsion to be above 98° C. for more than 5 min. Then these cooked acidified emulsions were chilled to below 6° C. for at least 10 min, then tempered to room temperature, and finally evaluated by a texture analyzer in the same way as in Step 4.

Analysis of the Samples and Results:
We evaluated the theory test samples T #2A, T #2B, T #2C, T #2D and T #2E with a texture analyser equipped with a 36 mm diameter cylinder shaped detection probe (model “P/36R”, 36 mm Radius Edge Cylinder probe), which pressed the suspension or curd-lie gel (from Step 3) twice from the top to 65% strain (compression depth, also known as compression distance, was 65% of the original height of the sample). Typical texture profile analysis (also known as “TPA”) was conducted on the compression tests, which projected values such as hardness, springiness, cohesiveness and resilience. Before the analysis, the samples were tempered to room temperature around 20° C. The compressing system including the detection probe, base cup-and-platform and sample was as shown in FIG. 4. The results are shown in Table 11 and in FIG. 15.

FIG. 15 shows textural properties measurements results for Theory test samples T #2A, T #2B, T #2C, T #2D and T #2E of Experiment III-1-C. The effect of the intensity of the disrupting during coagulation and the amount of acid on the gel strength of the coagulum. The samples were:
Theory Test Sample T #2A: pea protein 10%, 3.3% vinegar, non-disrupted-coagulation;
Theory Test Sample T #2B: pea protein 10%, 3.3% vinegar, disrupted-coagulation;
Theory Test Sample T #2C: pea protein 10%, 13.2% vinegar, non-disrupted-coagulation;
Theory Test Sample T #2D, pea protein 10%, 13.2% vinegar, disrupted-coagulation.

TABLE 11
Texture analysis results of compression tests
conducted on coagulated emulsion gel samples. The amount of
sample materials in each analysis cup was 150 g.

| | Hardness | Spring- | Cohesive- | Chewi- |
|---|---|---|---|---|
| Sample#, Main differences | (g) | iness | ness | ness |
| T#2A, Pea protein 10%, 3.3% vinegar, non-disrupted- | 263 | 94% | 29% | 71 |
| coagulation | | | | |
| T#2B, Pea protein 10%, 3.3% vinegar, disrupted-coagulation | 111 | 97% | 65% | 69 |
| T#2C, Pea protein 10%, 13.2% vinegar, non-disrupted- | 707 | 89% | 41% | 257 |
| coagulation | | | | |
| T#2D, Pea protein 10%, 13.2% vinegar, disrupted-coagulation | 576 | 72% | 45% | 186 |
| T#2E, Pea protein 12.5%, 3.3% vinegar, disrupted-coagulation, | 595 | 87% | 37% | 189 |
| cooking after acidification | | | | |

The Theory Test Sample T #2C having 13.2% vinegar and non-disrupted-coagulation treatment could be regarded as relatively mostly mimicking the tofu production process, in terms of complete coagulating at pH 4.6 (close to the pI, isoelectric point of the protein), and keeping the coagulated coagulum unbroken, or stable (non-disturbing, non-disrupting) during coagulation. The resulting sample has the higher hardness, gumminess and chewiness than the other samples in Table 11. This sample is clearly a gel, more specifically, a typical protein-based emulsion gel. Theory Test Sample T #2D had coagulation-disrupting treatment (disrupted-coagulation, conducted by cluster-disrupting mixing, stirring or kneading) clearly had lowered hardness, springiness, gumminess and chewiness of the gel; but, on the other hand, increased the cohesiveness and resilience. The effect of disrupted-coagulation on improving cohesiveness was more significant, more effective, when the vinegar addition dosage was 3.3% (cohesiveness increase from 29% in Theory Test Sample T #2A to 65% in Theory Test Sample T #2B), than when the vinegar dosage was 13.2% (cohesiveness increase from 41% in Theory Test Sample T #2C to 45% in Theory Test Sample T #2D). On the other hand, lowered acid dosage decreased the cohesiveness (cohesiveness decreased from 41% in Theory Test Sample T #2C to 29% in Theory Test Sample T #2A).

It was observed that high cohesiveness value in this test was in good correlation with observed free-flowing characteristics. This shows that there is a synergistic effect between cluster-disrupting mixing, stirring or kneading, and controlling of pH of the emulsion to be between pH 5.0 and 5.9, or 0.5-1.4 in the pH scale above the isoelectric point of the at least one plant-based proteinaceous ingredient. By combining these two factors (controlling the pH and cluster-disrupting), the coagulation can be more effectively and permanently disturbed. Either of the two factors used alone would not be as effective as the combined use of the two factors.

It is clear that the cooked disturbed-coagulum (Theory Test Sample T #2E) resulted in a very much weaker structure than “cooked patties” measured in similar way (such as Sample #1). This shows that the proteinaceous binder ingredient itself does not make a strong patty (or a strong formed meat-replacement product that is meat-imitate) or strong gel, even if it is cooked, but as a binder for TVPs in suitable piece form and manufactured with low moisture protein texturization extrusion, it makes a strong formed meat-replacement product that is meat-imitate.

The disturbed-coagulum with and without cooking after acidification (such as Theory Test Sample T #2A and T #2E) were not tofu, as they were too bad quality (texture) to be regarded as a commercially acceptable tofu. We also tested that, even when these samples were drained use cheese cloth, the remaining particles would not bind to each other to form tofu-like curd.

The Theory Test Sample T #2A (with coagulation-disrupting treatment) and T #2B (without coagulation-disrupting treatment) having 3.3% vinegar, were significantly weaker than Theory Test Sample #2C and #2D having 13.2% vinegar, correspondingly. This clearly reveals the relationship between decreased amount of acid addition and reduced gel strength. Less acid addition results in a higher pH, and results in lower completeness of coagulation, and eventually results in a weaker gel strength, in terms of hardness, gumminess and chewiness.

FIG. 15 shows that the samples (Theory Test Sample T #2B and T #2D) involving a coagulation-disrupting treatment (that was carried out during coagulation and started before the coagulation was substantially completed) had clearly more smooth and gradual increase of force along with increase of time between 0 s and 4 s than the samples without coagulation-disturbing treatment (Theory Test Sample T #2A and T #2C).

In contrast, the samples T #2A and T #2C without coagulation-disturbing had firstly a steep increase in force in the first second, approximately 5 mm compression depth (there was a steady compression speed 5 mm/s, so the compression depth can be calculated by multiplying the speed with compression time), then a slower increase or even a decrease between time 1 s and 2 s, and finally an increase between time 2 s and 4 s. This can be regarded as a “surface breaking” event, meaning that the surface of the gel was intact, and it took certain intensive force to break it. This reveals that the coagulated emulsion without coagulation-disturbing treatment is a more brittle and harder gel, while the coagulated emulsion with coagulation-disturbing treatment was a weaker and smoother gel.

The results reveal synergistic effects of (a) selecting a range of pH that is 0.5-1.4 above the isoelectric point, such as selecting pH 5.0-5.9 for using pea protein isolate; and (b) disturbing the coagulation. When the pH is between 5.0 and 5.9 (such as sample with 3.3% vinegar addition), the disturbing during coagulation (before the coagulation is completed to a plateau level, coagulation disruption during and/or soon enough after acid addition) was more effective in breaking gel structure of the overall suspension, for example, the disturbing during coagulation reduced the hardness of the gel by 60% (from 263 g to 111 g), and reduced the positive area by 60% (from 783 g×sec to 320 g×sec). On the other hand, when the pH is around 4.6 (sample with 13.2% vinegar addition), the disturbing during coagulation was less effective, reduced the hardness of the gel by 20% (from 707 g to 576 g), and reduced the positive area by 41% (from 1801 g×sec to 1057 g×sec). The possible reason for this will be explained below.

Difference Aspect #4, Particular Consistency (Gel Strength) of the Unit Particles in the Suspension or Gel,

detected by compressing the suspension or gel to a residue or remaining or final thickness of 0.8 mm, which is smaller or around the particle size of the self-encapsulated binder particles. Therefore, this compression result is different from those which were detecting the consistency of the whole suspension or gel, by compressing the whole suspension to a final thickness above 5 mm, in other words, only pressing the surface of middle depth of the gel.

Experiment III-1-D on Theory Test Samples T #1A and T #1B.

Differences in gel strength of different types of suspensions at different strain of compression, which reveal particular consistency (gel strength) of the unit particles in the suspension or gel.

We followed the Experiment III-1-C, and prepared Theory Test Samples T #1A and T #1B.

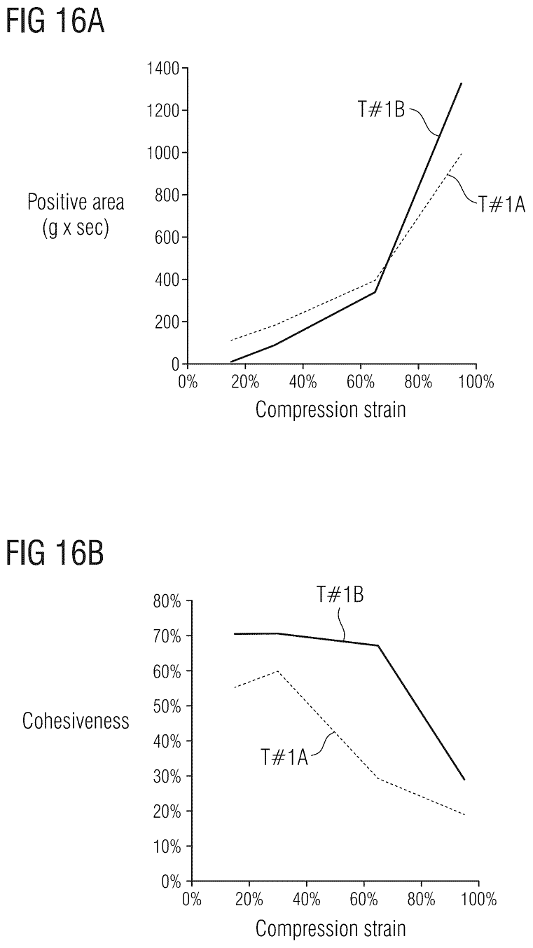

The texture analysis settings were different. We evaluated the theory test samples T #1A and T #1B, with a texture analyser equipped with a 36 mm diameter cylinder shaped detection probe (model “P/36R”, 36 mm Radius Edge Cylinder probe, Aluminium), which pressed the suspension/gel/coagulum twice from the top, to different strain values, such as 15%, 30%, 65% and 95% (so the compression depth, also known as compression distance, were, respectively, 15%, 30%, 65% and 95% of the original height of the samples). Each sample was tested at different strain levels in a sequence of strain 15%, followed by strain 30%, strain 65% and eventually strain 95%. Before the analysis, the samples were tempered to room temperature around 20° C. The samples were analysed as being contained in the same cup as in Step 2. The amount of samples (suspensions) in each cup was 50 g. Typical texture profile analysis (also known as “TPA”) was conducted on each compression test at a selected strain level. The results are shown in FIG. 16A. The tests were carried out by compressing the slurry binder system in a cup, 50 g of slurry in a cup, being coagulated with and without disturbing. The slurry was 15 mm thick, was compressed to different remaining thickness like 12.8 mm (strain 15%), 10.5 mm (strain 30%), 5.3 mm (65% strain) and 0.8 mm (95% strain).

FIG. 16A shows texture analysis measurement results, positive area value for Theory Test Samples T #1A and T #1B of Experiment III-1-D and illustrates differences in gel strength of different types of suspensions at different compressing strain. FIG. 16A shows that the suspension containing self-encapsulated binder particles 40 made by disrupted coagulation (Sample T #1B) is at low compression a significantly weaker gel than the curd-like gel made by non-disrupted coagulation (Sample T #1A), until compression strain around 65%. With further increase of compression strain, such as increased to 95%, the compression result start to be more reflecting particular consistency (gel strength) of the unit particles in the suspension or gel, the suspension containing self-encapsulated binder particles made by disrupted coagulation (Sample #2B) turned out to be stronger gel particles. This means that the suspension containing self-encapsulated binder particles (Theory Test Sample #2B) is a unit system having multiple level structure and texture, which is a weak gel and free-flowing suspension, and containing strong small gel particles, which are substantially evenly distributed.

FIG. 16B shows texture analysis measurement results, cohesiveness value for Theory Test Samples T #1A and T #1B of Experiment III-1-D, illustrating further differences in gel strength of different types of suspensions at different compressing strain. The suspension containing self-encapsulated binder particles made by disrupted-coagulation (Sample T #2B) had higher cohesiveness values than the other sample (Sample T #2A) at all compression strain levels. The cohesiveness values of the suspension containing self-encapsulated binder particles (Sample T #2B) decreased faster in the range of compression strain level between 65% and 95%, than in the range between 15% and 65%. This indicated that the irreversible deforming of the structure of the gel particles (gelling units of the suspension system) starts to occur more severely when it is compressed to a strain level 95%, with a resulting remaining thickness of the suspension as 0.8 mm.

Difference Aspect #5, Phase Stability, and/or Water Holding Properties, and/or Miscibility with Water.

Suspension containing self-encapsulated binder particles made by disrupted coagulation have good phase stability, that has no or little sediment, no or little water (whey) syneresis (e.g. less than 20%, preferably less than 10%, more preferably less than 5%). Nevertheless, there are visible water phase surrounding the self-encapsulated binder particles, in visual observation, the water phase is clear and transparent. If Suspension containing self-encapsulated binder particles is further suspended in water, e.g. in 1:1 ratio, the new mixture (suspension-water mixture) can keep stable non-separating phases for 12 hours or longer, no or practically no sedimentation of particles occurs.

The traditionally coagulated gel, such as tofu, normally shows a bad phase stability after the coagulation treatment, the gel shrinks, the water syneresis is heavy (e.g. more than 21%, mostly more than 30%). And if tofu curd-like gel is blended into small particles and suspended in water, e.g. in 1:1 ratio, they completely sediment to the bottom of the mixture within 8 hours, more frequently within 2 hours.

The homogenized gel, such as plant-based yogurt or our reference sample of homogenized gel, have even better good phase stability, that has no sediment, no or very little water (whey) syneresis (e.g. less than 5%, more frequently less than 1%). But as different from the suspension containing self-encapsulated binder particles made by disrupted coagulation, the homogenized gel does NOT have visible water phase surrounding the self-encapsulated binder particles, in visual observation, instead, the homogenized gel had a very homogenous appearance.

### III-2. Comparison Between Gelling Properties of Pea Protein and Soy Protein Emulsions

Theory test samples T #3A, T #3B, T #3C:

Sample Preparation:
Step 1. Prepare proteinaceous matrix:
   Step 1.a. Weighing pea protein isolate or soy protein isolate, plant based liquid oil, and water, in different ratios as: for Theory test sample T #3A, soy protein isolate 10% (as-is weight), oil 5.0% (as-is weight), water 85% (as-is weight) for; for Theory test sample T #3B, soy protein isolate 5% (as-is weight), oil 5.0% (as-is weight), water 90% (as-is weight); for Theory test sample #3C, pea protein isolate 10% (as-is weight), oil 5.0% (as-is weight), water 85% (as-is weight). Disperse these ingredients with a disperser, such as kitchen hand-held immersion blender, equipped with rotating knife blades, or other homogenizators. The blended mixture is a protein-based oil-in-water emulsion (“emulsion” hereafter). Fill the dispersed coagulated emulsion in a porcelain baking cup having a cylinder inner shape, diameter of 90 mm, height/depth 55 mm. All the quantities of the ingredients were determined so that the total weight in each Porcelain baking cup was 150 g.
   Step 1.b. Cook said emulsion with autoclave oven at 115° C. for 10 min, in order to reach temperature of the emulsion to be above 95° C., preferably above 98° C., for more than 2 min, preferably more than 5 min. As a result, the cooked emulsion (“cooked emulsion” hereafter) is made from this step.
Step 2. chill the cooked emulsion to 6° C. for at least 10 min.
Step 3. Evaluate the cooked emulsion with texture analyser. Before the analysis, the samples were tempered to room temperature around 20° C.

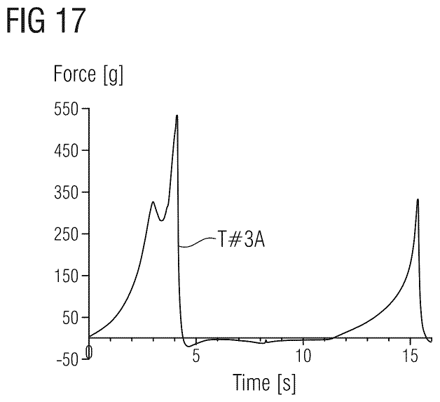

Instrumental Analysis and Results:
We evaluated the theory test samples T #3A, T #3B and T #3C with a texture analyser equipped with a 36 mm diameter cylinder shaped detection probe (model “P/36R”, 36 mm Radius Edge Cylinder probe, supplier Stable Micro Systems), which pressed the suspension or curd-like gel (from Step 3) twice from the top to 65% strain (compression depth, also known as compression distance, was 65% of the original height of the sample). Typical texture profile analysis (also known as “TPA”) was conducted on the compression tests, which projected values such as hardness, springiness, cohesiveness and resilience. The amount of sample materials in each analysis cup was 150 g. The compressing system including the detection probe, base cup-and-platform and sample was as shown in FIG. 4. Before the analysis, the samples were tempered to room temperature around 20° C. The results are shown in Table 12.
FIG. 17 shows the measurement results of Theory Test Sample T #3A, 10% soy protein. It shows that 10% soy protein emulsion became a typical gel, more specifically, protein based emulsion gel, that has hardness above 50 g, preferably above 200 g, more preferably above 300 g. This sample firstly had a steep increase in force in the first 3 second, followed by a decrease between time 3 second and 4 second, and finally a steep increase between time 4 second and 5 second. This can be regarded as a “surface breaking” event, meaning that the surface of the gel was intact, and it took certain intensive force to break it.

TABLE 12
Texture analysis results of compression tests
conducted on texture of cooked emulsion without
acidification. Comparing gelling properties of pea protein
and soy protein. The amount of sample materials in each
analysis cup was 150 g.

| Sample #, main differences | Hardness (g) | Springiness | Cohesiveness | Chewiness | Resilience |
|---|---|---|---|---|---|
| T#3A, 10% soy protein | 388 | 97% | 50% | 177 | 15% |
| T#3B, 5% soy protein | 7 | 100% | 100% | 7 | 0 |
| T#3C, 10% pea protein | 7 | 100% | 100% | 7 | 0 |

The results in Table 12 show that Theory Test Sample T #3A having 10% soy protein emulsion became a typical gel, more specifically, protein based emulsion gel, that has hardness above 50 g, preferably above 200 g, more preferably above 300 g.

On the other hand, the hardness of the Theory Test Sample T #3B made with soy protein isolate 5% and the Theory Test Sample T #3C made with pea protein isolate 5% were much lower, and were very low, approximately close to 8 g, mostly below 200 g, more typically 50 g.

These results show that the soy protein isolate is very different from non-soy protein, such as pea protein isolate. Soy protein isolate can form typical emulsion gel at conditions of protein content 10%, oil content 5% and cooking, in absence of acidification, while pea protein isolate is unable to do that. In other words, the non-soy proteins, such as pea protein isolate, have much weaker gel forming properties than soy protein.

Consequently, in order to achieve a similar gel strength of gels formed with soy protein isolate by using non-soy proteins, such as pea protein isolate, special treatment or very different gelling conditions are required. Such special treatment or very different gelling conditions can bring other unexpected influences to the properties and behaviour of the intermediate product during the manufacturing processes, and to the end product.

Compare to the results in Theory Test Sample T #2A and T #2C, the pea protein without acid coagulation, would not be able to make typical gels, while the acid coagulation is able to form weak gel or gel, which had hardness above 50 g, preferably above 200 g.

FIG. 17 shows that Theory Test Sample T #3A having 10% soy protein emulsion became a typical gel, more specifically, protein-based emulsion gel, that has hardness above 50 g, preferably above 200 g, more preferably above 300 g. This sample firstly had a steep increase in force in the first 3 second, followed by a decrease between time 3 second and 4 second, and finally a steep increase between time 4 second and 5 second. This can be regarded as a “surface breaking” event, meaning that the surface of the gel was intact, and it took certain intensive force to break it. On the other hand, Theory Test Samples T #3B and T #3C were too weak to have any detectable/displayable curve.

### III-3. Effect of Disrupted-Coagulation on Making Proteinaceous Binder from Soy Protein Instead of Pea Protein, Comparing Disrupted-Coagulation and Non-Disrupted-Coagulation

Theory Test Sample T #4A and T #4B:

Sample Preparation
Step 1. Prepare textured plant protein: in the same way as in Step 1 of Sample #1.
Step 2. Prepare proteinaceous matrix (“binder”):
Step 2.a. Weighing soy protein isolate, plant based liquid oil, and water, in ratio of soy protein 8% (as-is weight), oil 5% (as-is weight), water 87% (as-is weight). Disperse these ingredients with a disperser, such as kitchen hand-held immersion blender, equipped with rotating knife blades, or other homogenizators. The blended mixture is a protein-based oil-in-water emulsion (“emulsion” hereafter).
Step 2.b. Cook said emulsion with autoclave oven at 115° C. for 10 min, in order to reach temperature of the emulsion to be above 95° C., preferably above 98° C., for more than 2 min, preferably more than 5 min. As a result, the cooked emulsion (“cooked Emulsion” hereafter) is made from this step.
Step 2.c. After said emulsion is cooked (for samples require cooking step), before it is chilled to below 70° C., add vinegar to said cooked emulsion, the amount of the added vinegar is 3.3% of the cooked emulsion (as-is weight). For Theory test sample T #4A, the vinegar was mostly evenly distributed in the cooked emulsion by very gentle stirring and shaking, which did not disturb the coagulation process. It did not break most of the formed large protein clusters (size no less than 10 mm, preferably between 10 mm and 90 mm). As a result, Theory test sample T #4A was non-disturbed-coagulum, which is made by non-disrupted-coagulation from this step.
In contrary, for the other sample, such as Theory test sample T #4B, the coagulation was disturbed in the same way as in Step 2.c. of Sample #1, such as using cluster-disrupting mixing or stirring to disperse the mixture with spatula or whisk to break any formed large protein clusters (size no less than 10 mm, preferably between 10 mm and 200 mm) into smaller clusters or particles (size smaller than 10 mm, preferably smaller than 5 mm, more preferably smaller than 2 mm.) As a result, the dispersed coagulated emulsion (“Disturbed-coagulum” hereafter) is made from this step.
Step 3. Combine the textured plant protein made in Step 1, with the coagulated emulsion made in Step 2.c., in a ratio of 150 g textured plant protein and 300 g dispersed coagulated emulsion, and 10 g rapeseed oil.
Step 4. Mixing: Follow step 4 for Sample #1.
Step 5. Shape the kneaded mixture from Step 4, by packing the mixture in a vacuum bag with vacuum level 98%, then flatten the mixture to thickness of 15 mm, then take the sample out from vacuum, so the mixture is kept under vacuum for less than 10 min. This process is to mimic typical food industrial forming or shaping machine, such as plate mold forming machine (such as Forming Machine AFM from supplier Alco-food-machines GmbH & Co.), and vacuum assisted stuffing and forming machine. As a result, the shaped patty is made from this step.
Step 6. Evaluate some of the shaped patty from Step 5. with “three position compression bending test”.

Texture Analysis Method: three position compression bending test: The analysis method was the same three position compression bending test as in Experiment II-2.

TABLE 13
Texture analysis, three position bending test, results for bending compressing the shaped
but uncooked patties in a bending compressing test by a texture analyser Hardnes

| | Hardness | | | | | |
|---|---|---|---|---|---|---|
| Sample #, main differences | (g) | Adhesiveness | Springiness | Cohesiveness | Chewiness | Resilience |
| T# 4A, 8% soy protein, 5% oil, | 157 | −8 | 24% | 2% | 1.3 | 0% |
| 3.3% vinegar, Non-disrupted-coagulation | | | | | | |
| T# 4B, 8% soy protein, 5% oil, | 153 | −12 | 45% | 9% | 4.1 | 2% |
| 3.3% vinegar, Disrupted-coagulation | | | | | | |

Table 13 shows that the disrupted-coagulation treatment improved the shape stability of the shaped but uncooked patty made from soy protein. Theory test sample T #4B had clearly and largely increased springiness, cohesiveness and chewiness values of shaped but uncooked patty as compared to Theory test sample T #4A. We found that a sufficient quality of shaped and uncooked patty, namely, resistant and elastic enough against pressing and bending, is a necessary-but-insufficient condition for the successful industrial production of plant-based formed meat-replacement food product. Such quality is favourable and pivotal for product quality, production quality and capacity in industrial food processing, and less restrictive for the production process and operation. The shaped and uncooked patty should desirably have good stability and intactness of shape after shaping and before cooking, so can be more easily kept in good shape and quality in a normal and common industrial manufacturing (processing) environment that almost unavoidably have possible shaking, moving, pressing and bending forces being applied on the shaped products, such as at the phases between different processing steps, and at the transferring spaces between different machinery units (such as different processors and different conveying systems). Therefore, the disrupted-coagulation treatment had advantageous effects and improved the quality of manufacturing plant-based formed products—even when soy-based proteinaceous ingredients are used as the binder ingredient.

It is obvious to the skilled person that, along with the technical progress, the basic idea of the invention can be implemented in many ways. The invention and its embodiments are thus not limited to the examples and samples described above but they may vary within the contents of patent claims and their legal equivalents.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated feature but not to preclude the presence or addition of further features in various embodiments of the invention.

### REFERENCE PUBLICATIONS CITED

[1] “Ingredients used in the preparation of canned foods”, by Susan Featherstone in “A complete Course in Canning and Related Processes (14^{th} ed.)”, 2015, available under https://www.sciencedirect.com/topics/agricultural-and-biological-sciences/textured-vegetable-protein
[2] Jiang Z. Q., Sontag-Strohm, T., Salovaara, H., Sibakov, J., Kanerva, P., & Loponen, J. (2015). Oat protein solubility and emulsion properties improved by enzymatic deamidation. Journal of Cereal Science, 64, 126-132)
[3] “TPA”, in page “Application Guide” of software Exponent (“Stable Micro Systems”), Version 6.1.11.0, Copyright 2007 Stable Micro Systems Ltd. All Rights Reserved. Stable Micro Systems Ltd, Vienna Court, Lammas Road, Godalming, Surrey, UK. GU7 1YL. Date of reviewing 2020.12.22. Date of original publishing of the document: unknown.
[4] “Overview of Texture Profile Analysis”, (website link: https://texturetechnologies.com/resources/texture-profile-analysis) Copyright belonging to (2020) Texture Technologies Corp. and Stable Micro Systems, Ltd.|Hamilton, MA 01982. Date of reviewing 2020.12.22. Date of original publishing of the document: unknown.

### LIST OF REFERENCE NUMERALS USED

1, 2 structure of curd-like gel made from non-disrupted-coagulation process
3 structure of proteinaceous binder ingredient
4 structure of coagulated and over-disrupted suspension
10 typical plant protein molecule, native and not denaturized
11 amino acid side chain having negative net charge
12 hydrophobic sites
13 Sulphur-containing amino acid sites
14 peptide chains
20 a typical heat denaturized plant protein molecule
30 heat denaturized protein molecule in unfolded shape under an environment that has pH value close to the pI (isoelectric point) of the proteins
31 highly ordered broad continuous network structure
40 self-encapsulated proteinaceous particle (=plant-based binder ingredient, may have paste-like consistency)
40′ self-encapsulated proteinaceous particle opened
40″ self-encapsulated proteinaceous particle after over-disrupted-coagulation
41 proteinaceous fiber
42 protein bundle
42′ bundle of protein fibres bonding with opened self-encapsulated proteinaceous particles
42″ TVP-particle-particle-TVP sandwich structure
45 water or aqueous liquid
49 suspension comprising self-encapsulated proteinaceous particles 40 and water/aqueous liquid 45 (the suspension 49 is also a plant-based proteinaceous binder ingredient)

1. A method of manufacturing a formed meat-replacement food product, comprising:
a) preparing a mixture of:
i) textured vegetable proteins manufactured using a low moisture protein texturization extrusion method that have a sponge-like texture and are in particulate, granule, flake, chunk, or thread form, and
ii) a plant-based proteinaceous binder ingredient excluding wheat-based proteinaceous ingredients and methylcellulose, which comprises a disturbed-coagulum that is a dispersion of a gel or a weak gel of an emulsion of:
a. water/water-containing liquid and lipid that preferably is fat, oil, or margarine, and
b. at least one plant based excluding wheat proteinaceous ingredient with the proteinaceous portion having at least 6.1% by weight of the binder ingredient,
b) shaping the mixture into desired form; and
c) processing the shaped mixture such that the binder ingredient undergoes further gelation under non-melting conditions for the proteins, wherein the processing includes one or more of cooking, frying, grilling, steaming or baking.

2. The method according to claim 1, wherein: between the mixing and shaping steps, the mixture is kneaded.

3. The method according to claim 1, wherein: a protein crosslinking/binding enzyme is added into the mixture.

4. The method according to claim 1, wherein: the plant-based proteinaceous binder ingredient comprises a protein colloid, protein extract, protein isolate, protein concentrate, or a mixture of at least two of these.

5. The method according to claim 1, wherein: the plant-based proteinaceous binder ingredient comprises protein fractions having isoelectric points between 4 and 5.

6. The method according to claim 1, wherein: the plant-based proteinaceous binder ingredient comprises protein fractions having water solubility being affected by pH, and the lowest solubility is at pH between 4 and 5.

7. The method according to claim 1, wherein: the plant-based proteinaceous binder ingredient comprises protein fractions having gel forming properties after being denatured and acidified to pH between 3.5 and 6.0, or to 0.5-1.4 in the pH scale above the isoelectric point of the at least one plant-based proteinaceous ingredient.

8. The method according to claim 1, wherein: the plant-based proteinaceous binder ingredient comprises materials selected from the group consisting of: cereal grains (excluding wheat), legume crops, vegetable crops, pulse grains, oilseeds and seeds for sowing forage.

9. The method according claim 8, wherein the cereal grains comprise one or more of the following: oat, barley, rye, rice, or corn.

10. The method according to claim 8, wherein the pulse grains, legume crops, vegetable crops, oilseeds, or seeds for sowing forage comprise one or more of the following: peas, beans, green peas, peanuts, chickpeas, fava beans, lentils, kidney beans, white beans, black beans, black eye beans, mung beans, soy beans, or alfalfa.

11. The method according to claim 1, wherein: the plant-based proteinaceous binder ingredient is a suspension having a pH between 5.0 and 5.9.

12. The method according to claim 1, wherein: in the mixing step, also a protein crosslinking/binding enzyme is included; the mixing is carried out below 40° C.

13. The method according to claim 12, wherein: the time between the mixing step and the shaping step is less than 4 h.

14. The method according to claim 12, wherein: in the method, between the mixing step and the shaping step, the temperature of the mixture is kept below 40° C.

15. The method according to claim 12, wherein: the shaped mixture is subjected to an incubation step, in which the shaped product is
i) incubated at temperature above 30° C. for a time between 30 min and 4 hours, or
ii) incubated at temperature between 1° C. and 6° C., for a time between 4 h and 36 h.

16. The method according to claim 15, wherein: the processing step is a direct continuation to the incubation step, after heating the shaped product in an autoclave oven to an incubation temperature above 30° C., for a suitable incubation time between 30 min and 4 h, then directly further increasing the temperature to the processing temperature 115° C.

17. A formed meat-replacement food product manufactured using the method according to claim 1.

18. The formed meat-replacement food product according to claim 17, wherein: the food product is devoid of gluten, egg protein, meat protein, dairy, methylcellulose, wheat and soy.

19. The formed meat-replacement food product according to claim 17, wherein: the formed meat-replacement food product is a vegetarian/vegan meat-replacement patty, a vegetarian/vegan meat-replacement ball, a vegetarian/vegan meat-replacement sausage, a vegetarian/vegan meat-replacement fish finger, a vegetarian/vegan meat-replacement ham, or a vegetarian/vegan meat-replacement nugget.

20. A method for manufacturing a plant-based proteinaceous binder ingredient, comprising the steps of:
Mixing an emulsion of
a water or water-based liquid,
lipid that is fat, oil, or margarine, comprising one or more of the following: oilseed oil, rapeseed oil, field mustard oil, olive oil, coconut fat, or cocoa fat, and
at least 6.1% by weight of at least one plant-based proteinaceous ingredient excluding wheat-based proteinaceous ingredients and methylcellulose that comprises, at least one protein colloid, extract, isolate, or concentrate or a mixture of at least two of these;
Converting the mixture to a gel or weak gel by heating and a coagulation-causing treatment with acidifying in such a manner that its pH is between 5.0 and 5.9 or 0.5-1.4 in the pH scale above the isoelectric point of the at least one plant-based proteinaceous ingredient;
Disrupting the coagulation to obtain disturbed-coagulum before the coagulation is completed.

21. The method according to claim 20, wherein: the disruption of the coagulation is carried out to break protein clusters in the gel by cluster-disrupting mixing, stirring or kneading with a spatula or a whisk during the coagulation.

22. The method according to claim 20, wherein: the disrupting is limited in intensity or duration such that disrupting is ended before disrupted-coagulation goes over to homogenization.

23. The method according to claim 20, wherein: the coagulation-causing treatment comprises the step of adding acid to achieve a pH between 5.0 and 5.9, at a temperature between 50° C. and 100° C. which is carried under coagulation-disrupting dispersion.

24. The method according to claim 20, wherein: the coagulation-causing treatment comprises the steps of adding acid to achieve a pH between 5.0 and 5.9, at a temperature between 50° C. and 100° C., followed by coagulation-disrupting dispersion within 24 h before the mixture is chilled to below 10° C.

25. The method according to claim 20, wherein: in the disrupting step the size of any large gel clusters is controlled-reduced in size to a predetermined range.

26. The method according to claim 20, wherein: gel clusters having a size of at least 10 mm are reduced by dispersing in size substantially to size larger than 0.101 mm and smaller than 10 mm.

27. The method according to claim 20, wherein: the coagulation-causing treatment includes the step of adding vinegar, citrus fruit juice, acetic acid, lactic acid, or any weak acid with a pKa value between 2.0 and 5.1, or a substance substantially containing a weak acid which has a pKa value between 2.0 and or any mixture thereof.

28. The method according to claim 20, wherein: i) the at least one plant-based proteinaceous ingredient comprises protein fractions having isoelectric points between 4 and 5 or ii) the at least one plant-based proteinaceous ingredient comprises one or more of: pea proteins, fava bean proteins, mung bean proteins, or chickpea proteins.

29. The method according to claim 20, wherein: the at least one plant-based proteinaceous ingredient comprises protein fractions having water solubility being affected by pH, and the lowest solubility is at a pH between 4 and 5.

30. The method according to 20, wherein: the at least one plant-based proteinaceous ingredient comprises protein fractions having gel forming properties after being denatured and acidified to a pH between 3.5 and 6.0, or to 0.5-1.4 in the pH scale above the isoelectric point of the at least one plant-based proteinaceous ingredient.

31. The method according to claim 20, wherein: the plant-based proteinaceous ingredient comprises one or more of: cereal grains (excluding wheat), legume crops, vegetable crops, pulse grains, oilseeds or seeds for sowing forage.

32. The method according to claim 31, wherein: the cereal grains comprise one or more of the following: oat, barley, rye, rice, or corn; or some or all of the cereal grains have been treated with protein-glutaminase induced deamidation reaction to deamidation degree approximately between 50% and 70%, to achieve a status that the proteins substantially comprise proteins having isoelectric points between 3.5 and 6.0.

33. The method according to claim 31, wherein the pulse grains legume crops vegetable crops oilseeds, or seeds for sowing forage one or more of the following: peas, beans, green peas, peanuts, chickpeas, fava beans, lentils, kidney beans, white beans, black beans, black eye beans, mung beans, soy, or alfalfa; or the at least one plant-based proteinaceous ingredient comprises pea proteins, fava bean proteins, mung bean proteins, chickpea proteins, or any combination thereof.

34. The method according to claim 20, wherein: the method further comprises the step of adding a protein crosslinking/binding enzyme.

35. The method according to claim 34, wherein: the protein crosslinking/binding enzyme is added at or after the disrupting step.

36. The method according to ag claim 20, further comprising the step of: after or at the disrupting step, mixing with water-absorbing materials, that are dry materials, to change the consistency to paste-like.

37. A plant-based proteinaceous binder ingredient manufactured with a method according to claim 20 comprising: a disturbed-coagulum of
an emulsion of:
water or water-based liquid and lipid that is fat, oil, or margarine comprising one or more of the following: oilseed oil, rapeseed oil, field mustard oil, olive oil, coconut fat, or cocoa fat, and
at least 6.1 by weight of at least one plant-based proteinaceous ingredient excluding wheat-based proteinaceous ingredients and methylcellulose that comprises one or more of protein colloid extract isolate concentrate, or mixture.

38. A plant-based proteinaceous binder ingredient according to claim 37, wherein: substantially all gel clusters of the gel or weak gel after the disruption have a size larger than 0.101 mm and smaller than 10 mm.

39. The plant-based proteinaceous binder ingredient according to claim 37, wherein: the at least one plant-based proteinaceous ingredient comprises protein fractions having isoelectric points between 4 and 5.

40. The plant-based proteinaceous binder ingredient according to claim 37, wherein: the at least one plant-based proteinaceous ingredient comprises protein fractions having water solubility being affected by pH, and the lowest solubility is at a pH approximately between 4 and 5.

41. The plant-based proteinaceous binder ingredient according to claim 37, wherein: the at least one plant-based proteinaceous ingredient comprises protein fractions having gel forming properties after being denatured and acidified to a pH between 3.5 and 6.0, or to 0.5-1.4 in the pH scale above the isoelectric point of the at least one plant-based proteinaceous ingredient.

42. The plant-based proteinaceous binder ingredient according to claim 37, wherein: the plant-based proteinaceous binder ingredient is a suspension having a pH between 5.0 and 5.9.

43. The plant-based proteinaceous binder ingredient according to claim 37, wherein: the plant-based proteinaceous ingredient comprises one or more of the following: cereal grains (excluding wheat), legume crops, vegetable crops, pulse grains, oilseeds, or seeds for sowing forage.

44. The plant-based proteinaceous binder ingredient according to claim 43, wherein the cereal grains comprise one or more of the following: oat, barley, rye, rice, or corn; or some or all of the cereal grains have been treated with protein-glutaminase induced deamidation reaction to deamidation degree approximately between 50% and 70%, to achieve a status that the proteins substantially comprise proteins having isoelectric points between 3.5 and 6.0.

45. The plant-based proteinaceous binder ingredient according to claim 43, wherein the pulse grains, legume crops, vegetable crops, oilseeds, or seeds for sowing forage comprise one or more of the following: peas, beans, green peas, peanuts, chickpeas, fava beans, lentils, kidney beans, white beans, black beans, black eye beans, mung beans, soy beans, or alfalfa.

46. The plant-based proteinaceous binder ingredient according to claim 37, wherein: the at least one proteinaceous binder ingredient has been incompletely coagulated by protein denaturation, followed by acidification mixing with vinegar, citrus fruit juice, acetic acid, lactic acid, or any weak acid with a pKa value between 2.0 and 5.1, or a substance substantially containing a weak acid which has a pKa value between 2.0 and 5.1.

47. A method for manufacturing self-encapsulated proteinaceous particles, comprising the steps of:
Denaturizing proteinaceous particles in a suspension that is an emulsion containing i) lipids, ii) water or any liquid substantially consisting of water, and iii) at least one plant-based proteinaceous material, excluding methylcellulose;
Inducing gelling or weak gelling in the suspension; and
Disrupting the coagulation to obtain disturbed-coagulum before the coagulation is substantially completed, such that self-encapsulated protein particles are formed.

48. The method according to claim 47, wherein: during the coagulation, pH and temperature of the suspension are controlled.

49. The method according to claim 48, wherein: the pH is controlled such that the suspension is 0.5-1.4 in the pH scale above the isoelectric point of the at least one plant-based proteinaceous ingredient.

50. The method according to claim 47, wherein: the disrupting is ended before disrupted-coagulation goes over to homogenization.

51. The method claim 47, wherein: the disruption is carried out to effect gel clusters having a size of at least 10 mm, to be reduced by dispersing in size substantially to sizes larger than 0.101 mm and smaller than 10 mm.

52. The method according to claim 47, wherein: the suspension is dried to a paste-like consistency.

53. Self-encapsulated proteinaceous particles manufactured with the method of claim 47, comprising: gel clusters having a size larger than 0.101 mm and smaller than 10 mm.

54. The self-encapsulated proteinaceous particles according to claim 53, wherein: the particles do not show binding to the other particles when they are pressed to touch each other by draining or centrifugal dehydrating.

55. Self-encapsulated proteinaceous particles manufactured with the method of claim 47, wherein: the particles in a suspension are a unit system having a multiple level structure and texture, which is a weak gel and free-flowing suspension, and containing strong small gel particles, which are substantially evenly distributed.

56. The self-encapsulated proteinaceous particles according to claim 55, comprising: in a suspension, a visible water phase surrounding the self-encapsulated binder particles.

57. A method of using self-encapsulated proteinaceous particles according to claim 53, wherein: the self-encapsulated particles are introduced into a mixture containing solid matter, the mixture is mixed or kneaded causing shear forces, such that the shear forces open the self-encapsulated proteinaceous particles in such a form that the outer surface of the self-encapsulated proteinaceous particles bonds with the solid matter.

58. The method according to claim 57, wherein: the mixture is coagulated to create a crosslinked matrix within the whole mixture.

59. A method of using self-encapsulated proteinaceous particles according to claim 53, wherein: the self-encapsulated particles are used to encapsulate lipids inside the gel clusters for storing them air-tightly.

60. The method of claim 59, wherein: the lipids comprise fat-soluble aroma substances and/or fat-soluble spices, introduced into the self-encapsulated particles from emulsion.

61. A method of using the plant-based proteinaceous binder ingredient of claim 1 in an industrial manufacturing process of formed meat-replacement products, wherein the proteinaceous binder ingredient comprises a disturbed-coagulum.

62. A method of improving the stability of self-encapsulated proteinaceous particles, comprising: producing a proteinaceous suspension as an emulsion; and treating the emulsion to produce disturbed-coagulum containing self-encapsulated particles.

63. The method according to claim 62, wherein: the emulsion comprises lipids of an oil, fat, or margarine comprising one or more of the following: oilseed oil, rapeseed oil, field mustard oil, olive oil, coconut fat, or cocoa fat.

64. The method of claim 1, further comprising:
one or more of the following: oilseed oil, rapeseed oil, field mustard oil, olive oil, coconut fat, or cocoa fat.

65. The method of claim 64 wherein the at least one plant based proteinaceous ingredient with the proteinaceous portion is between 6.1% to 15% of the binder ingredient.

66. The method of claim 64 wherein the at least one plant based proteinaceous ingredient with the proteinaceous portion is between 7% and 15% by weight of the binder ingredient.

67. The method of claim 1, further comprising one or more of the following: potato, onion, tomato, herbs, spices, salt, breadcrumbs, or starches, wherein the potato is boiled, baked, powdered, or raw.

68. The method of claim 67 wherein the potato, onion, tomato, herbs, spices, salt, breadcrumbs, or starches, are dried.

69. The method of claim 1 wherein transglutaminase is added into the mixture.

70. The method of claim 9 wherein at least one of the cereal grains have been treated with protein-glutaminase induced deamidation reaction to deamidation degree approximately between 50% and 70%, to achieve a status that the proteins substantially comprise proteins having isoelectric points between 3.5 and 6.0.
